(19) United States
(12) Patent Application Publication
(10) Pub. No.: US 2026/0335301 A1
(43) Pub. Date: Oct. 29, 2026

(54) COMBINATION THERAPIES INCLUDING METAL CHANNEL ACTIVATORS AND TDP-43 MODULATORS

(71) Applicant: Biohaven Therapeutics Ltd., Tortola (VG)

(72) Inventor: Vladimir CORIC, Madison, CT (US)

(21) Appl. No.: 19/108,169

(22) PCT Filed: Sep. 5, 2023
(86) PCT No.: PCT/US2023/073491

§ 371 Date: Mar. 2, 2025

(51) Int. Cl. A61K 31/472; A61K 31/325; A61K 31/4152; A61K 31/423; A61K 31/428; A61K 31/4406; A61K 31/4439; A61K 31/454; A61K 31/4545; A61K 31/4709; A61K 31/496; A61K 31/497; A61K 31/498; A61K 31/501; A61K 31/506; A61K 31/517; A61K 31/5377; A61K 31/541; A61K 38/06

(52) U.S. Cl. CPC A61K 31/472; A61K 31/325; A61K 31/4152; A61K 31/423; A61K 31/428; A61K 31/4406; A61K 31/4439; A61K 31/454; A61K 31/4545; A61K 31/4709; A61K 31/496; A61K 31/497; A61K 31/498; A61K 31/501; A61K 31/506; A61K 31/517; A61K 31/5377; A61K 31/541; A61K 38/06

(57) ABSTRACT

Provided is a pharmaceutical composition including a metal channel activator and a TDP-43 modulator. Also provided is a method of treating a depressive disorder, including administering the pharmaceutical composition. Also provided is a method of treating a neurological or neurodegenerative disorder, including administering the pharmaceutical composition. Also provided is a method of treating a pain disorder, including administering the pharmaceutical composition.

### CROSS-REFERENCE TO RELATED APPLICATIONS

This application is a national phase entry under 35 U.S.C. § 371 of International Application PCT/US2023/073491, filed Sep. 5, 2023, which claims priority to U.S. Provisional Patent Application No. 63/492,547, filed Mar. 28, 2023, and U.S. Provisional Patent Application No. 63/403,988, filed Sep. 6, 2022, all of which are herein incorporated by reference in their entirety.

### TECHNICAL FIELD

The present invention relates to combination therapies for treatment of various medical conditions. Specifically, the present invention relates to a combination of a metal channel activator and a modulator of the trans-activation response element (TAR) DNA-binding protein 43 (TDP-43) for treatment of neurological and neurodegenerative disorders. In particular, the metal channel activator may target the Kv7 family of voltage-gated potassium (K⁺) channels.

### BACKGROUND

Metal channel activators (openers) are associated with a wide range of physiological functions including the regulation of the electrical properties of excitable cells. Metal channels control the follow of metal ions such as Potassium (K⁺) and Sodium (Na⁺) across a cell membrane. A primary function of these channels in the brain is to regulate the neuronal action potential. Several neurologic disorders are potentially due to dysregulation of metal channels.

Potassium (K⁺) channels, present on the plasma membranes of most cell types, are the most diverse class of all ion channels. Potassium channels of the Kv7 family of voltage-gated potassium (K⁺) channels are of particular therapeutic interest due to their importance in neurological conditions such as excitability disorders including Amyotrophic lateral sclerosis (ALS). There are five members of the Kv7 family of voltage-gated potassium (K⁺) channels, including Kv7.1, Kv7.2, Kv7.3, Kv7.4, and Kv7.5.

Metal channel activators have been reported to be useful for treatment of various neurological and neurodegenerative disorders. Thus far, only one metal channel activator, Retigabine, has been FDA approved. Retigabine is used as anticonvulsant for the treatment of epilepsy. Further Kv7 channel activators have been proposed for the treatment of many conditions including substance abuse and mood disorders (Vigil F A, Carver C M, Shapiro M S. Pharmacological Manipulation of Kᵥ7 Channels as a New Therapeutic Tool for Multiple Brain Disorders. Front Physiol. 2020 Jun. 19; 11:688. doi: 10.3389/fphys.2020.00688). There remains a need, however, for new therapies utilizing Kv7. One solution is combining metal channel activators with other therapeutic agents to significantly improve treatment outcomes.

TDP-43 (also known as TARBP and more formally known as transactive response DNA binding protein 43 kDa) is a multifaceted regulator of transcription and translation. TDP-43 enacts this function through both RNA and DNA binding domains. Although only a subset of patients with ALS have a genetic mutation related to TDP-43, aggregates of TDP-43 have been identified in 97% of ALS cases. These aggregates may be due to post translational modifications including ubiquitination, phosphorylation, acetylation, PARylation, and methylation, or due to mislocalization away from the nucleus. Counteracting these multifaceted aggregates is a major therapeutic objective for ALS.

More generally, a pathological form of the transactive response (TAR) DNA binding protein (TDP-43) is known to bind to RNA in stress granules to form membraneless, amyloid-like TDP-43 aggregates in the cytoplasm of ALS motor neurons. Such aggregates cause RNA dysregulation, which contributes to disease pathogenesis of amyotrophic lateral sclerosis (ALS) and other neurologic or neurodegenerative diseases. TDP-43 binders are known to prevent binding of TDP-43 to RNA, so they may be potentially useful to stop or at least slow down progression of ALS or another neurologic or neurodegenerative disease in a patient.

Current treatment options for ALS and other neurological or neurodegenerative disorders are limited. For example, Riluzole is an approved treatment for ALS. Riluzole is believed to have anti-excitotoxic method of action, in part, through an anti-glutaminergic/Na+ blocking pathway. However, hyperexcitability may be a consequence of increased Na+ or decreased K+ conductance depending on age and disease stage. Analysis of K+ channels in sporadic ALS postmortem spinal cords suggests hypermethylation, down-regulation, and decreased K+ channel expression, suggesting a dysregulated potassium current in ALS pathology. Several aspects are not clear, such as whether hyperexcitability is intrinsic or synaptic, and how long it persists.

It is disclosed that both modulating K+ channels and preventing binding of TDP-43 to RNA as a result of a treatment regimen will more effectively treat ALS or other neurologic or neurodegenerative diseases. Therefore, administering a K+ channel modulator (or other metal ion channel modulator) in combination with a TDP-43 modulator will have a greater benefit than either therapy administered alone. As an alternative to a TDP-43 modulator, modulators of protein targets other than TDP-43 may be utilized which are effective to disaggregate TDP-43 or treat TDP-43 proteinopathies. Additionally, one or more K+ channel activators or TDP-43 modulators may have a dose-dependent adverse effect limiting their therapeutic window. However, the combination of a K+ channel activator and TDP-43 modulator will allow for use of lower doses of one or more of the K+ channel activator or TDP-43 modulator, to achieve improved treatment efficacy while reducing adverse effects, if any. It is disclosed that a Kv7 activator in combination with a TDP-43 modulator will more effectively treat, prevent, and/or slow the progression of ALS or other neurologic or neurodegenerative diseases compared to current available treatments.

Described herein are compositions and methods for combination therapies of Kv7 openers with TDP-43 modulators. The combination therapies have treatment capabilities greater than Kv7 openers or TDP-43 modulators alone, and may be particularly useful for neurological diseases. Pharmaceutical compositions combining these agents may also be useful for treatment of a pain or depressive disorder. These two agents target distinct molecular targets associated with neurological diseases and neurodegenerative disorders such as ALS. Specifically, the Kv7 channel opener reduces excitability, and TDP-43 modulators reduce aggregation of TDP-43. We have found that combining Kv7 opener and a TDP-43 modulator will yield improved treatment outcomes as described herein.

### SUMMARY

The present invention is directed to combination therapies including a metal channel activator and a TDP-43 modulator.

In an embodiment, provided is a pharmaceutical composition including a metal channel activator and a TDP-43 modulator.

In another embodiment, disclosed is a method for treating or preventing a disease that involves TDP-43, including administering to a subject the pharmaceutical composition.

In another embodiment, disclosed is a method for treating or preventing a disease associated with TDP-43 proteinopathies, including administering to a subject the pharmaceutical composition.

In another embodiment, disclosed is a method for treating or preventing diseases that involve excess amounts of TDP-43 in the cytosol, including administering to a subject the pharmaceutical composition.

### DETAILED DESCRIPTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

As used herein, when specific definition is not otherwise provided, the term “substituted” refers to a group substituted with deuterium, a halogen (—F, —Cl, —Br, —I), a hydroxy group (—OH), an amino group (—NH₂), a carboxyl group (—CO₂H), a substituted or unsubstituted C1-C10 amine group, a nitro group (—NO₂), a C1-C10 alkyl group, a C3-C10 cycloalkyl group, a C6-C12 aryl group, a C1-C10 alkoxy group, a C1 to C10 trifluoroalkyl group such as a trifluoromethyl group (—CF₃) and the like, or a cyano group (—CN) instead of at least one hydrogen of a substituting group or compound.

As used herein, “modulator”, “modulators”, and “modulating”, when describing compounds, describe compounds that may enact their effect through a number of mechanisms of action including but not limited to: binding to the active site of a protein, binding to a region of the protein away from the active site, causing relocalization of a protein, inducing degradation of a protein, inducing stabilization of a protein, causing a conformational change in a protein, decreasing the activation threshold for a protein, increasing the activation threshold for a protein, altering posttranslational modifications for a protein, reducing the transcription of a gene, increasing the transcription of a gene, reducing the translation of an mRNA transcript, increasing the translation of an mRNA transcript, disrupting an interaction between two proteins, and stabilizing an interaction between two proteins; wherein the protein may be the target of modulation or an intermediary protein which is associated with modulation of the target protein. Modulators of the targets described herein (i.e., Kv7 and TDP-43) may each or both be small molecule compounds, proteins, antibody fragments or antibodies, or any other construct effecting modulation.

As used herein, “TDP-43 modulator” may target TAR DNA binding protein 43 (TDP-43), or other human proteins known in the art to modulate TDP-43, including but not limited to mitochondrial permeability transition pore (mPTP), phosphatidylinositol-3-phosphate 5-kinase type III (PIKfyve), Cytochrome P450 Family 51 Subfamily A Member 1 (CYP51A1), and Forkhead Box O (FOXO) family proteins. TDP-43 modulators may target any other human protein modulating or effecting modulation of TDP-43, as appreciated by a person of skill in the art.

As used herein, “neurological disease” refers to a disease or disorder which affects the brain and/or nerves found elsewhere in the body. Such neurologic diseases may include: Absence of the Septum Pellucidum, Acid Lipase Disease, Acute Disseminated Encephalomyelitis, Adrenoleukodystrophy, Agenesis of the Corpus Callosum, Agnosia, Aicardi Syndrome, Aicardi-Goutieres Syndrome, Alexander Disease, Alpers' Disease, Alternating Hemiplegia, Alzheimer's Disease, Amyotrophic Lateral Sclerosis (ALS), Anencephaly, Angelman Syndrome, Antiphospholipid Syndrome, Aphasia, Apraxia, Arachnoid Cysts, Arachnoiditis, Arteriovenous Malformation, Asperger Syndrome, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Back Pain, Barth Syndrome, Batten Disease, Behcet's Disease, Bell's Palsy, Benign Essential Blepharospasm, Binswanger's Disease, Brachial Plexus Injuries, Brain and Spinal Tumors, Brown-Sequard Syndrome, CADASIL, Canavan Disease, Carpal Tunnel Syndrome, Central Cord Syndrome, Central Pain Syndrome, Central Pontine Myelinolysis, Cephalic Disorders, Cerebellar Degeneration, Cerebellar Hypoplasia, Cerebral Aneurysms, Cerebral Arteriosclerosis, Cerebral Atrophy, Cerebral Cavernous Malformation, Cerebral Hypoxia, Cerebral Palsy, Cerebro-Oculo-Facio-Skeletal Syndrome (COFS), Charcot-Marie-Tooth Disease, Chiari Malformation, Chorea, Chronic Inflammatory Demyelinating Polyneuropathy (CIDP), Chronic Pain, Coffin Lowry Syndrome, Colpocephaly, Coma, Complex Regional Pain Syndrome, Congenital Myasthenia, Congenital Myopathy, Corticobasal Degeneration, Craniosynostosis, Creutzfeldt-Jakob Disease, Cushing's Syndrome, Dandy-Walker Syndrome, Deep Brain Stimulation for Movement Disorders, Dementia, Dementia With Lewy Bodies, Dermatomyositis, Developmental Dyspraxia, Diabetic Neuropathy, Dravet Syndrome, Dysautonomia, Dysgraphia, Dyslexia, Dyssynergia Cerebellaris Myoclonica, Dystonia, Empty Sella Syndrome, Encephalitis Lethargica, Encephaloceles, Encephalopathy, Epilepsy, Erb-Duchenne and Dejerine-Klumpke Palsies, Essential Tremor, Fabry Disease, Fahr's Syndrome, Familial Periodic Paralyses, Farber's Disease, Febrile Seizures, Fibromuscular Dysplasia, Foot Drop, Friedreich Ataxia, Frontotemporal Dementia, Functional Neurologic Disorder, Gaucher Disease, Generalized Gangliosidoses, Gerstmann's Syndrome, Gerstmann-Straussler-Scheinker Disease, Giant Axonal Neuropathy, Glossopharyngeal Neuralgia, Guillain-Barre Syndrome, Headache, Hemicrania Continua, Hemifacial Spasm, Hereditary Neuropathies, Hereditary Spastic Paraplegia, Herpes Zoster Oticus, Holmes-Adie Syndrome, Holoprosencephaly, Huntington's Disease, Hydranencephaly, Hydrocephalus, Hydromyelia, Hypersomnia, Hypertonia, Hypotonia, Inclusion Body Myositis, Incontinentia Pigmenti, Infantile Neuroaxonal Dystrophy, Infantile Refsum Disease, Infantile Spasms, Inflammatory Myopathies, Iniencephaly, Isaacs' Syndrome, Joubert Syndrome, Kearns-Sayre Syndrome, Kennedy's Disease, Kleine-Levin Syndrome, Klippel-Feil Syndrome, Klippel-Trenaunay Syndrome (KTS), Klüver-Bucy Syndrome, Krabbe Disease, Kuru, Lambert-Eaton Myasthenic Syndrome, Landau-Kleffner Syndrome, Learning Disabilities, Leigh's Disease, Lennox-Gastaut Syndrome, Lesch-Nyhan Syndrome, Leukodystrophy, Lipid Storage Diseases, Lipoid Proteinosis, Lissencephaly, Locked-In Syndrome, Machado-Joseph Disease and Spinocerebellar Ataxia, Megalencephaly, Melkersson-Rosenthal Syndrome, Meningitis and Encephalitis, Menkes Disease, Meralgia Paresthetica, Metachromatic Leukodystrophy, Microcephaly, Migraine, Miller Fisher Syndrome, Mitochondrial Myopathies, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia Gravis, Myoclonus, Myopathy, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Sequelae Of Lupus, Neuromyelitis Optica, Neuronal Migration Disorders, Neurosarcoidosis, Neurosyphilis, Neurotoxicity, Niemann-Pick Disease, Normal Pressure Hydrocephalus, Occipital Neuralgia, Ohtahara Syndrome, Olivopontocerebellar Atrophy, Opsoclonus Myoclonus, Orthostatic Hypotension, Paraneoplastic Syndromes, Paresthesia, Parkinson's Disease, Paroxysmal Choreoathetosis, Paroxysmal Hemicrania, Parry-Romberg Syndrome, Pelizaeus-Merzbacher Disease, Peripheral Neuropathy, Periventricular Leukomalacia, Pervasive Developmental Disorders, Pinched Nerve, Piriformis Syndrome, Pituitary Tumors, Polymyositis, Pompe Disease, Porencephaly, Post-Polio Syndrome, Postural Tachycardia Syndrome, Primary Lateral Sclerosis, Progressive Multifocal Leukoencephalopathy, Progressive Supranuclear Palsy, Prosopagnosia, Pseudotumor Cerebri, Rasmussen's Encephalitis, Refsum Disease, Repetitive Motion Disorders, Restless Legs Syndrome, Rett Syndrome, Reye's Syndrome, Sandhoff Disease, Schilder's Disease, Schizencephaly, Septo-Optic Dysplasia, Shaken Baby Syndrome, Shingles, Sjögren's Syndrome, Sleep Apnea, Sotos Syndrome, Spasticity, Spina Bifida, Spinal Cord Infarction, Spinal Cord Injury, Spinal Muscular Atrophy, Stiff-Person Syndrome, Striatonigral Degeneration, Stroke, Sturge-Weber Syndrome, Subacute Sclerosing Panencephalitis, SUNCT Headache, Swallowing Disorders, Sydenham Chorea, Syncope, Syringomyelia, Tabes Dorsalis, Tardive Dyskinesia, Tarlov Cysts, Tay-Sachs Disease, Tethered Spinal Cord Syndrome, Thoracic Outlet Syndrome, Thyrotoxic Myopathy, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis Syndromes of the Central and Peripheral Nervous Systems, Von Hippel-Lindau Disease (VHL), Wallenberg's Syndrome, Wernicke-Korsakoff Syndrome, Whiplash, Whipple's Disease, Williams Syndrome, Wilson Disease, and Zellweger Syndrome.

As used herein, the term “metal channel activator” is construed to include both metal channel activator and pharmaceutically acceptable salt thereof.

As used herein, the term “TDP-43 modulator” is construed to include both TDP-43 modulator and pharmaceutically acceptable salt thereof.

As used herein, the term “AUC” is the definite integral of the concentration of a drug in blood, cerebrospinal fluid, target organ, or any other physiologically relevant site after a dose is given as a function of time. AUC in this context is used to measure the total exposure to the drug across time. AUC can be evaluated over a definite time interval or estimated based on the integral drug concentrations measured over a time interval extrapolated to infinite time.

As used herein, the term “Cmax” is the highest concentration of a drug in the blood, cerebrospinal fluid, target organ, or any other physiologically relevant site after a dose is given.

As used herein, the term “Tmax” is the time taken after administration for a drug to reach its highest concentration in the blood, cerebrospinal fluid, target organ, or any other physiologically relevant site after a dose is given.

As used herein, the term “proteinopathies” refers to diseases characterized by an accumulation or aggregation of a single protein, or multiple proteins. Proteinopathies include but are not limited to: Creutzfeldt-Jakob disease and other prion diseases, Alzheimer's disease, Parkinson's disease, amyloidosis, multiple system atrophy, Amyotrophic lateral sclerosis (ALS), Frontotemporal Lobar Degeneration, and Lewy Body Dementia.

Additional aspects will be set forth in part of the description which follows and, in part, will be apparent from the description.

In the formulas set forth below, it is set out with substituents and definitions, and it should be clear to the reader that substituents and definitions are numbered (e.g., R¹, R², Y, etc.) and are intended to apply within a given formula. Numberings that are repeated across formulas are intentional and should be each read within the context of each particular formula.

### Metal Channel Activators

Amyotrophic lateral sclerosis (ALS) is a neurological disorder characterized by progressive upper and lower motor neuron degeneration, leading to muscular atrophy and eventually fatal paralysis. A now appreciated characteristic of ALS is hyperexcitability of motor neurons (Wainger B J, Kiskinis E, Mellin C, Wiskow O, Han S S, Sandoe J, Perez N P, Williams L A, Lee S, Boulting G, Berry J D, Brown R H Jr, Cudkowicz M E, Bean B P, Eggan K, Woolf C J. Intrinsic membrane hyperexcitability of amyotrophic lateral sclerosis patient-derived motor neurons. Cell Rep. 2014 Apr. 10; 7(1): 1-11. doi: 10.1016/j.celrep.2014.03.019. Epub 2014 Apr. 3. PMID: 24703839; PMCID: PMC4023477). Mutations in metal channels, and in particular mutations in the Kv7 family of voltage-gated potassium (K⁺) channels have been implicated in hyperexcitability disorders (Jepps T A, Barrese V, Miceli F. Editorial: Kv7 Channels: Structure, Physiology, and Pharmacology. Front Physiol. 2021 Apr. 16; 12:679317. doi: 10.3389/fphys.2021.679317. PMID: 33935812; PMCID: PMC8085343). The Kv7 family includes Kv7.1-7.5 corresponding to the KCNQ1-5 genes. Kv7 channel activators, a subset of metal channel activators, can reduce hyperexcitability of neurons. This suggests that treatment of ALS patients with Kv7 channel activators may reduce ALS symptomology. Thus, in combination with TDP-43 as described herein, effective treatment outcomes are obtained for ALS.

Examples of metal channel activators including Kv7 channel activators are disclosed in Formulas 1-8 and in the corresponding referenced applications.

### Formula 1

In an embodiment, the Kv7 channel activator may be selected from one of the following compounds according to Formula 1. Such compounds are described in U.S. Pat. No. 9,481,653, issued Nov. 1, 2016, and corresponding to U.S. application Ser. No. 14/853,815 filed Sep. 14, 2015; and US Publication No 20210188782A1, published Jun. 24, 2021, and corresponding to U.S. application Ser. No. 17/127,231 filed Dec. 18, 2020; which are incorporated by reference in their entirety herein. In the case of any conflict of terminology in the context of Formula 1, these references incorporated by reference herein control.

In an embodiment, the Kv7 channel activator is a compound according to formula 1:

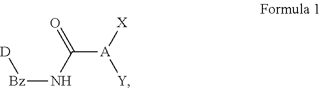

wherein D is optionally substituted C₃₋₆ carbocyclyl, optionally substituted C₂₋₅ heterocyclyl, isopropyl, or t-butyl; Bz is optionally substituted benzoimidazol-1,2-diyl; A is C₁₋₈ alkyl; X is F; and Y is H, F, Cl, Br, I, or a moiety having a molecular weight of 15 Da to 300 Da and consisting of 2 to 5 chemical elements, wherein the chemical elements are independently C, H, O, N, S, F, Cl, or Br.

In further embodiments, D is optionally substituted cyclobutyl, optionally substituted phenyl, optionally substituted isoxazolyl, optionally substituted pyridinyl, isopropyl, or t-butyl.

In further embodiments, D is optionally substituted cyclobutyl, optionally substituted phenyl, optionally substituted isoxazolyl, optionally substituted pyridinyl, isopropyl, or t-butyl, wherein each substituent of D and Y, if present, independently has a molecular weight of 15 Da to 200 Da and consists of 2 to 5 chemical elements, wherein the chemical elements are independently C, H, O, N, S, F, Cl, or Br.

In another embodiment, the Kv7 channel activator is a compound according to formula 1 wherein, D is optionally substituted C₃₋₆ carbocyclyl, optionally substituted C₂₋₅ heterocyclyl, isopropyl, or t-butyl; Bz is optionally substituted benzoimidazol-1,2-diyl; A is C₁₋₈ alkyl; X is H, F, CF₃, optionally substituted phenyl, or optionally substituted pyridinyl; and Y is H, F, Cl, Br, I, or a moiety having a molecular weight of 15 Da to 300 Da and consisting of 2 to 5 chemical elements, wherein the chemical elements are independently C, H, O, N, S, F, Cl, or Br.

In further embodiments, D is optionally substituted cyclobutyl, optionally substituted phenyl, optionally substituted isoxazolyl, optionally substituted pyridinyl, isopropyl, or t-butyl.

In further embodiments, each substituent of D, X, and Y, if present, independently has a molecular weight of 15 Da to 200 Da and consists of 2 to 5 chemical elements, wherein the chemical elements are independently C, H, O, N, S, F, Cl, or Br.

In further embodiments, R¹ is H, Cl, Br, CN, OCH₃, CF₃, —CO₂CH₂CH₃, C₁₋₄ alkyl, or C₁₋₄ hydroxyalkyl.

In further embodiments, X is optionally substituted phenyl.

In further embodiments, X is CF₃.

In further embodiments, X is optionally substituted pyridinyl.

In further embodiments, X is H.

In further embodiments, Y is H.

In further embodiments, Y is OH.

In further embodiments, the Kv7 channel activator is a compound selected from the group consisting of:

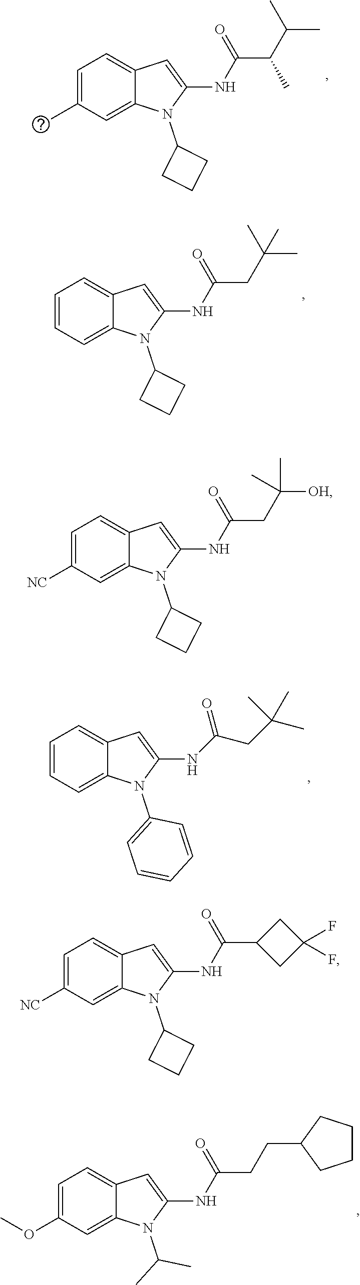

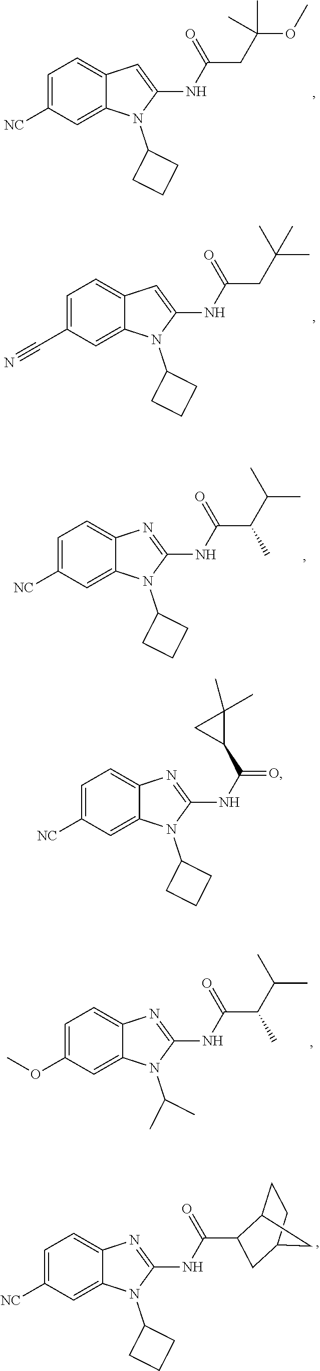

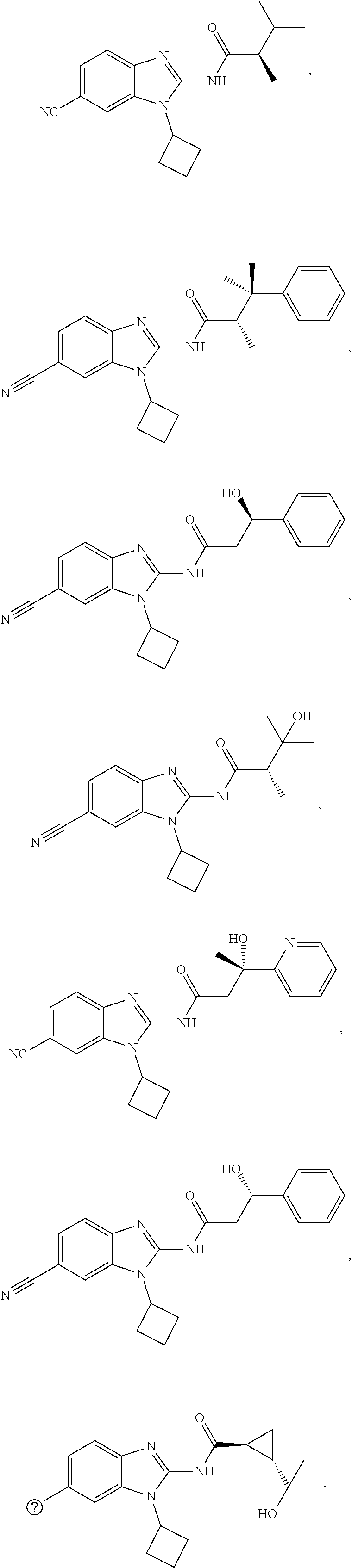

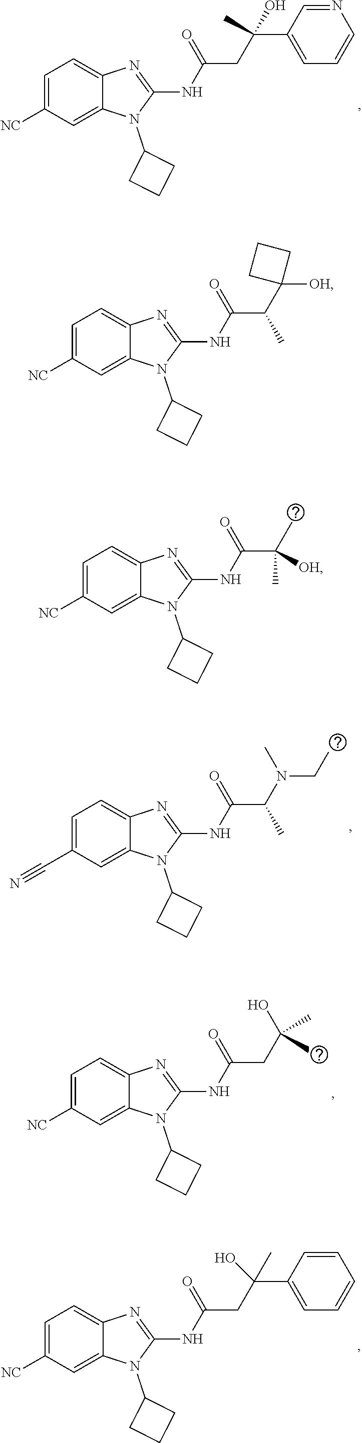

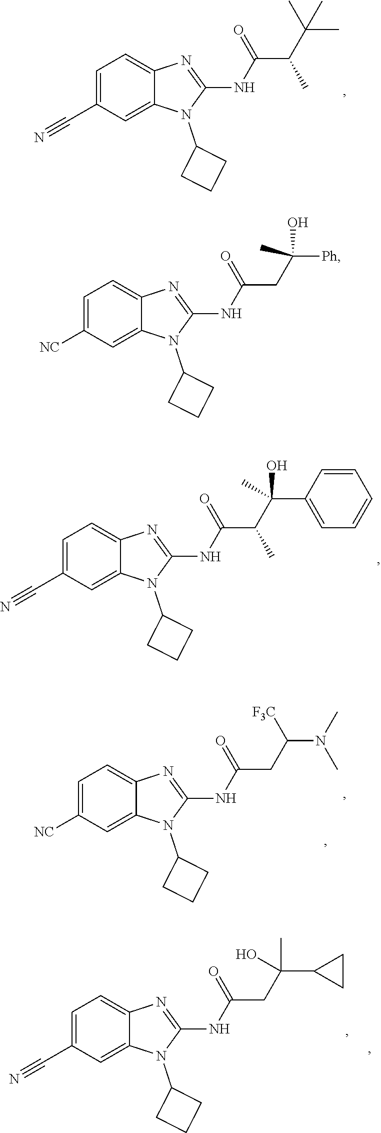 [SMILES: C[C@H](C(=O)Nc1nc2ccc(C#N)cc2n1C1CCC1)C(C)(C)C ; C[C@](O)(CC(=O)Nc1nc2ccc(C#N)cc2n1C1CCC1)c1ccccc1 ; C[C@H](C(=O)Nc1nc2ccc(C#N)cc2n1C1CCC1)[C@@](C)(O)c1ccccc1 ; CN(C)C(CC(=O)Nc1nc2ccc(C#N)cc2n1C1CCC1)C(F)(F)F ; CC(O)(CC(=O)Nc1nc2ccc(C#N)cc2n1C1CCC1)C1CC1]

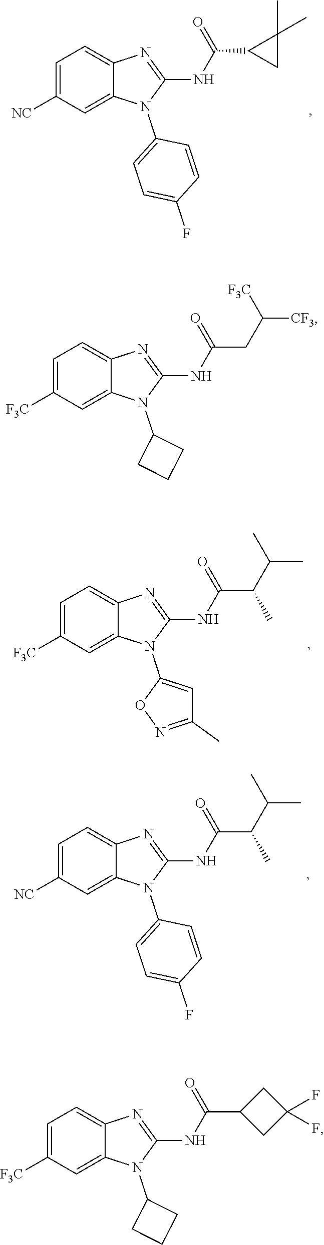

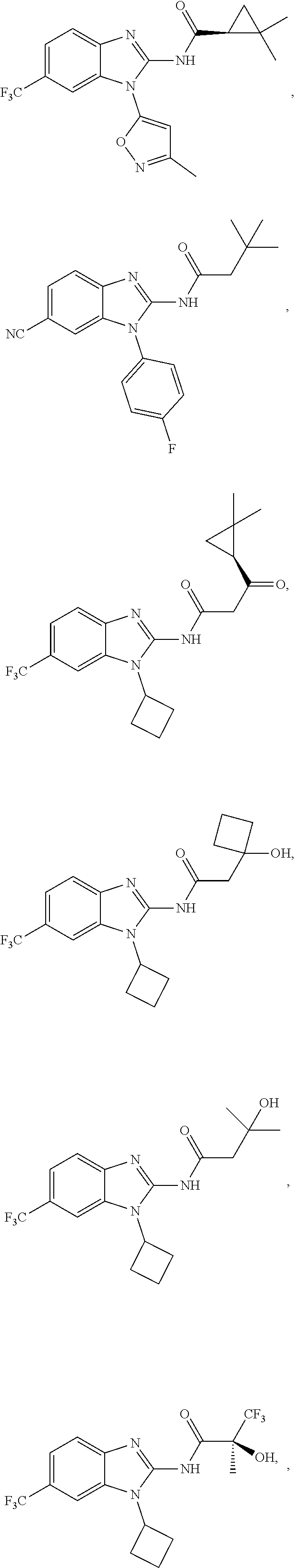 [SMILES: Cc1cc(-n2c(NC(=O)[C@H]3CC3(C)C)nc3ccc(C(F)(F)F)cc32)on1 ; CC(C)(C)CC(=O)Nc1nc2ccc(C#N)cc2n1-c1ccc(F)cc1 ; CC1(C)C[C@@H]1C(=O)CC(=O)Nc1nc2ccc(C(F)(F)F)cc2n1C1CCC1 ; O=C(CC1(O)CCC1)Nc1nc2ccc(C(F)(F)F)cc2n1C1CCC1 ; CC(C)(O)CC(=O)Nc1nc2ccc(C(F)(F)F)cc2n1C1CCC1 ; C[C@](O)(C(=O)Nc1nc2ccc(C(F)(F)F)cc2n1C1CCC1)C(F)(F)F]

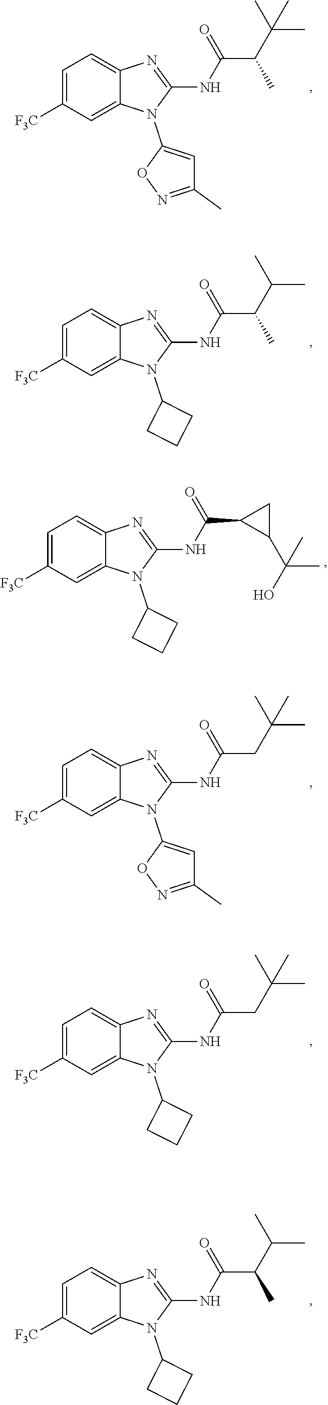 [SMILES: Cc1cc(-n2c(NC(=O)[C@@H](C)C(C)(C)C)nc3ccc(C(F)(F)F)cc32)on1 ; CC(C)[C@H](C)C(=O)Nc1nc2ccc(C(F)(F)F)cc2n1C1CCC1 ; CC(C)(O)C1C[C@@H]1C(=O)Nc1nc2ccc(C(F)(F)F)cc2n1C1CCC1 ; Cc1cc(-n2c(NC(=O)CC(C)(C)C)nc3ccc(C(F)(F)F)cc32)on1 ; CC(C)(C)CC(=O)Nc1nc2ccc(C(F)(F)F)cc2n1C1CCC1 ; CC(C)[C@@H](C)C(=O)Nc1nc2ccc(C(F)(F)F)cc2n1C1CCC1]

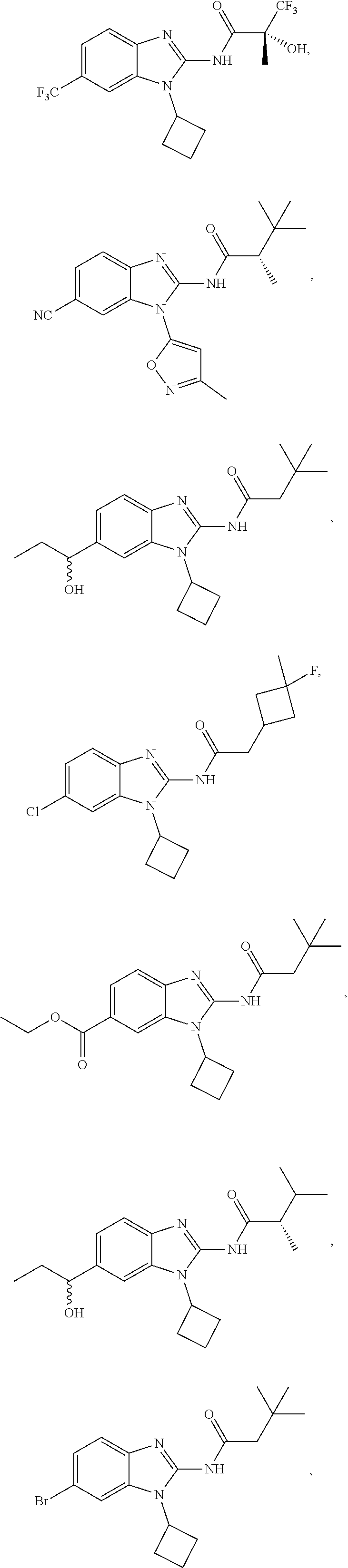

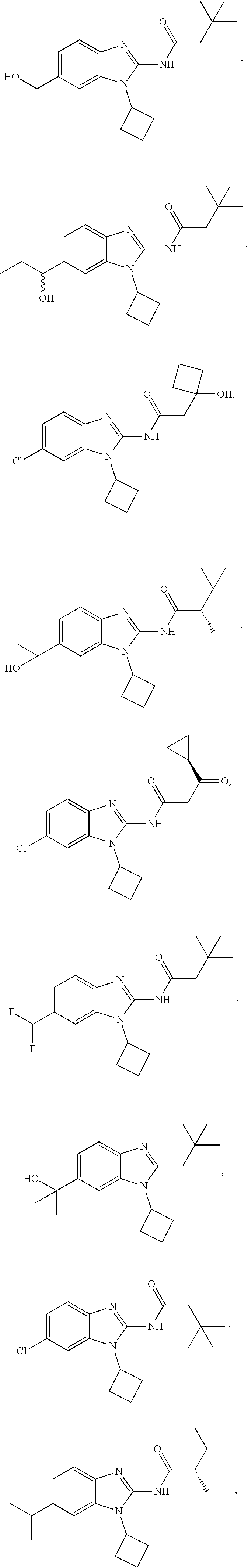

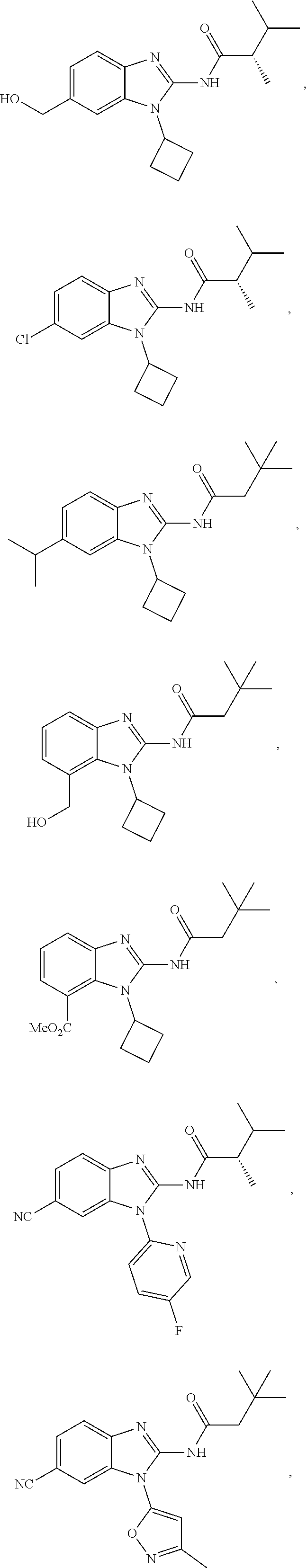

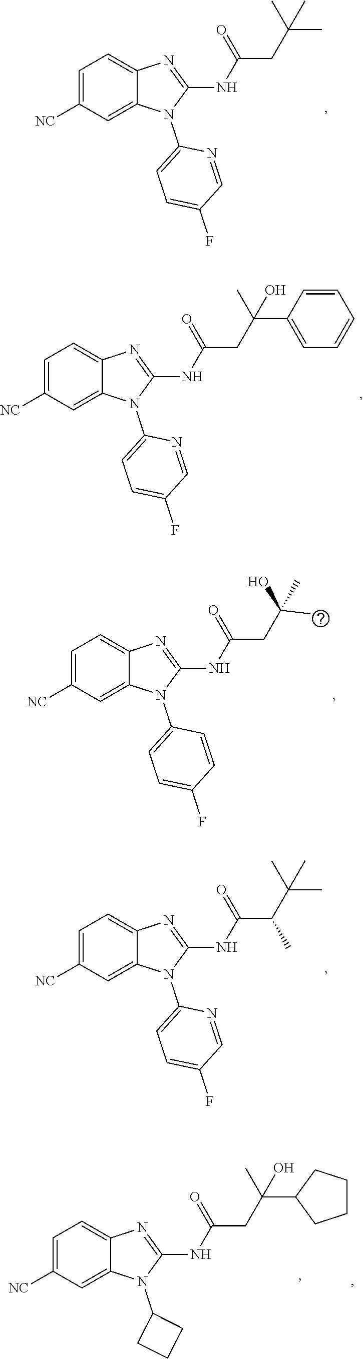

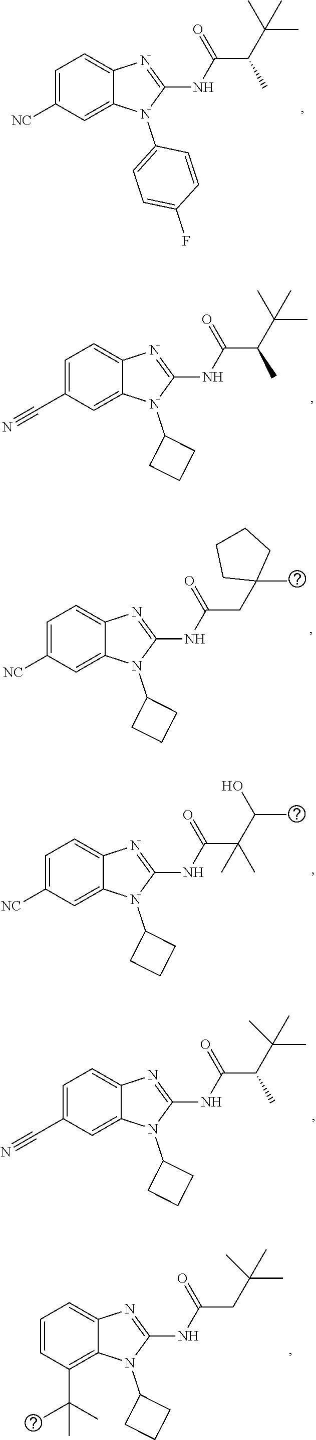 [SMILES: C[C@H](C(=O)Nc1nc2ccc(C#N)cc2n1-c1ccc(F)cc1)C(C)(C)C ; C[C@@H](C(=O)Nc1nc2ccc(C#N)cc2n1C1CCC1)C(C)(C)C ; CC1(CC(=O)Nc2nc3ccc(C#N)cc3n2C2CCC2)CCCC1 ; CC(O)C(C)(C)C(=O)Nc1nc2ccc(C#N)cc2n1C1CCC1 ; C[C@H](C(=O)Nc1nc2ccc(C#N)cc2n1C1CCC1)C(C)(C)C ; CC(C)(C)CC(=O)Nc1nc2cccc(C(C)(C)C)c2n1C1CCC1]

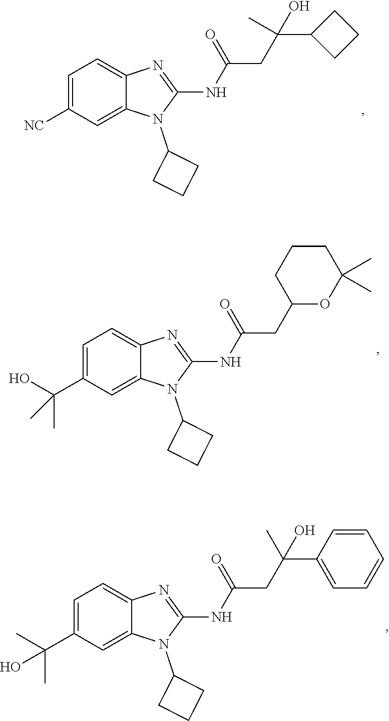

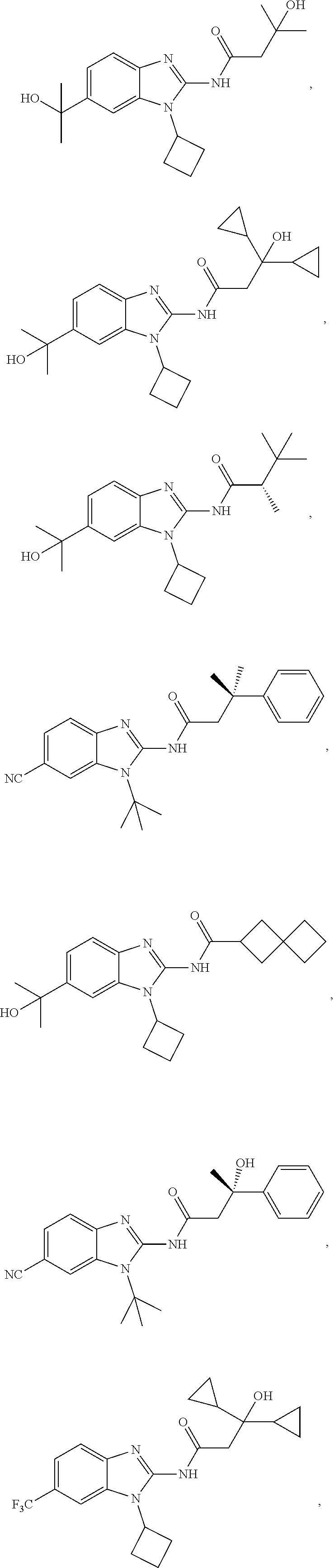

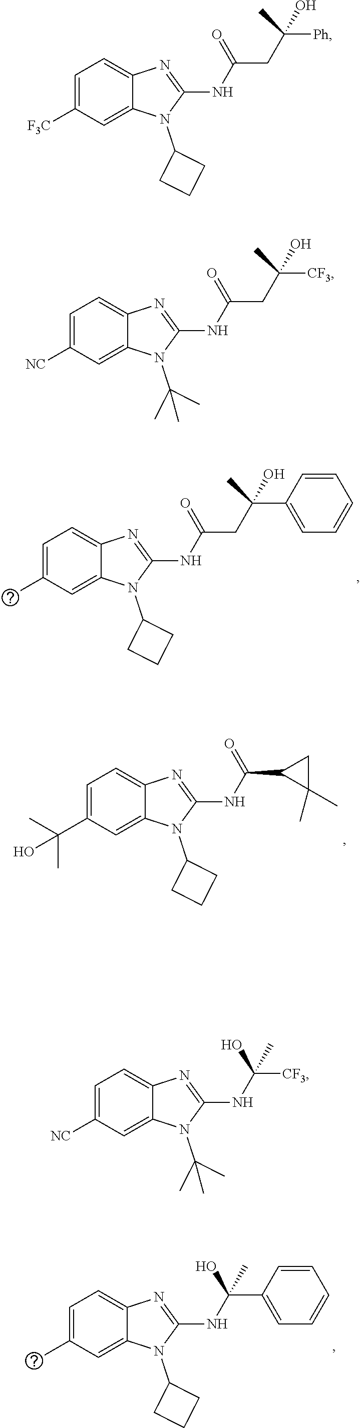

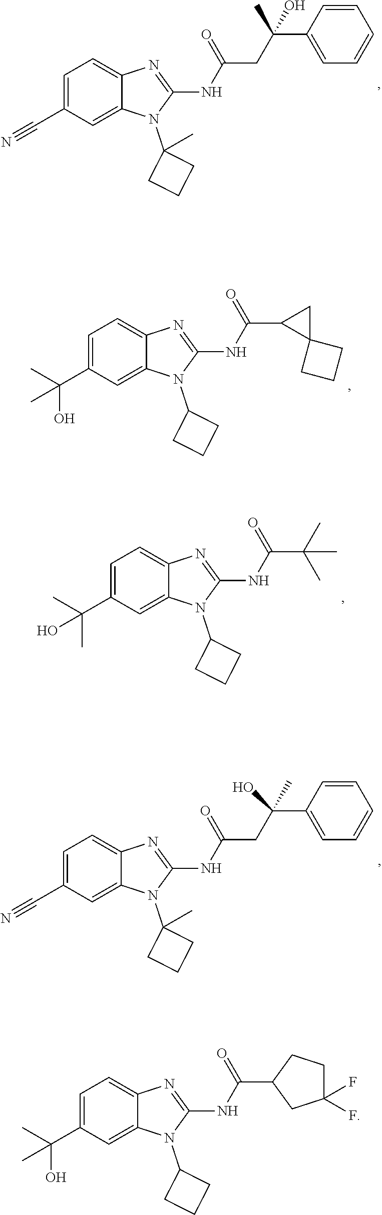

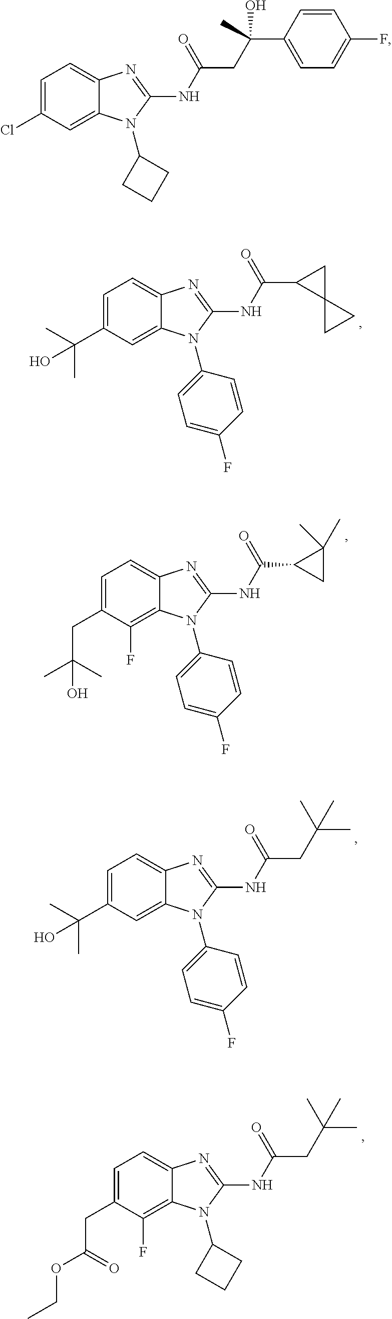

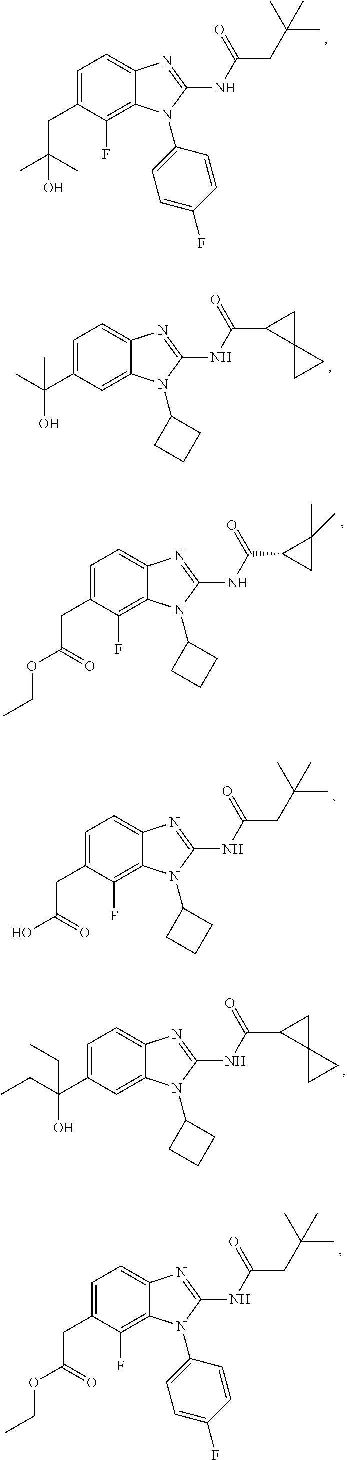

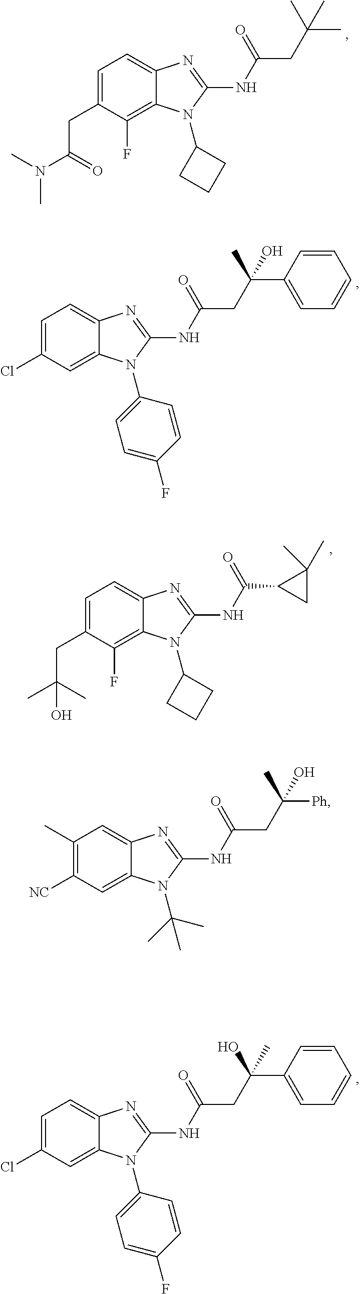

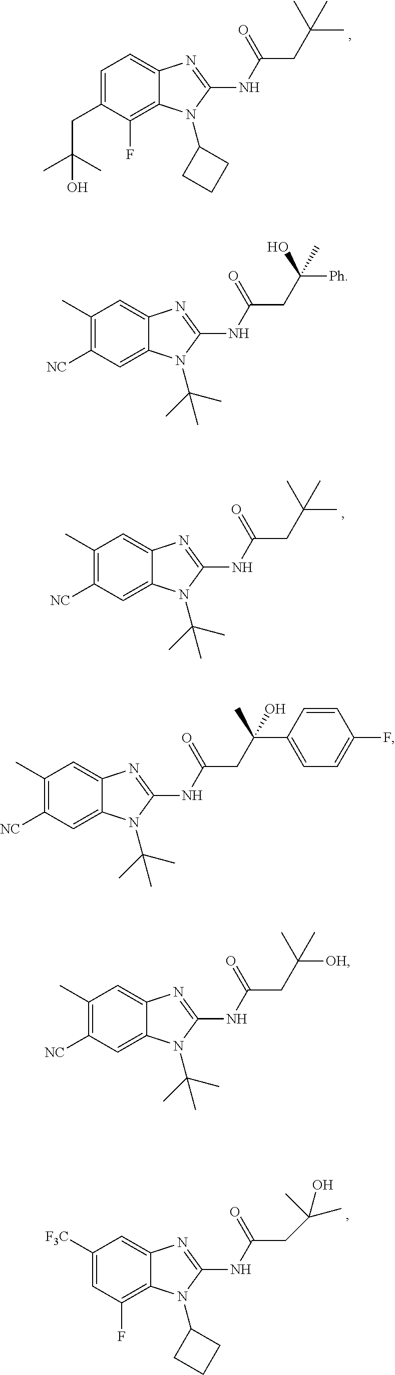

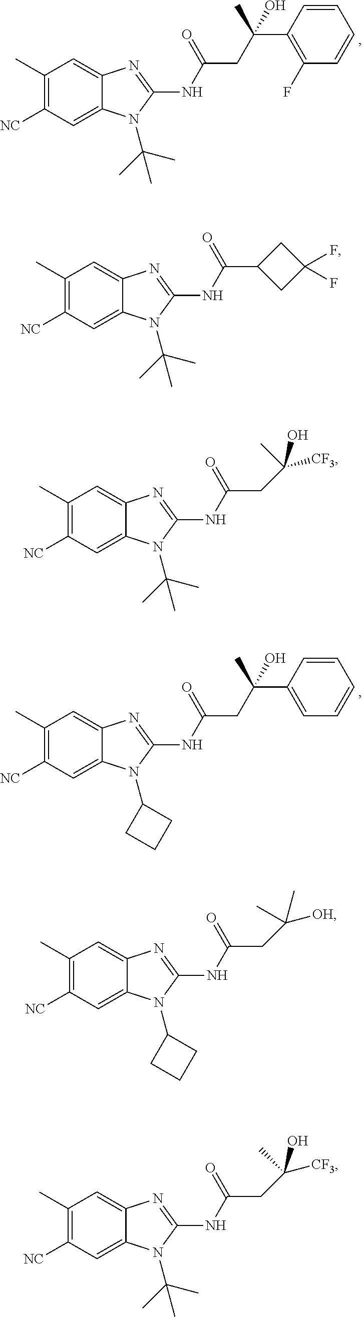 [SMILES: Cc1cc2nc(NC(=O)C[C@](C)(O)c3ccccc3F)n(C(C)(C)C)c2cc1C#N ; Cc1cc2nc(NC(=O)C3CC(F)(F)C3)n(C(C)(C)C)c2cc1C#N ; Cc1cc2nc(NC(=O)C[C@@](C)(O)C(F)(F)F)n(C(C)(C)C)c2cc1C#N ; Cc1cc2nc(NC(=O)C[C@](C)(O)c3ccccc3)n(C3CCC3)c2cc1C#N ; Cc1cc2nc(NC(=O)CC(C)(C)O)n(C3CCC3)c2cc1C#N ; Cc1cc2nc(NC(=O)C[C@@](C)(O)C(F)(F)F)n(C(C)(C)C)c2cc1C#N]

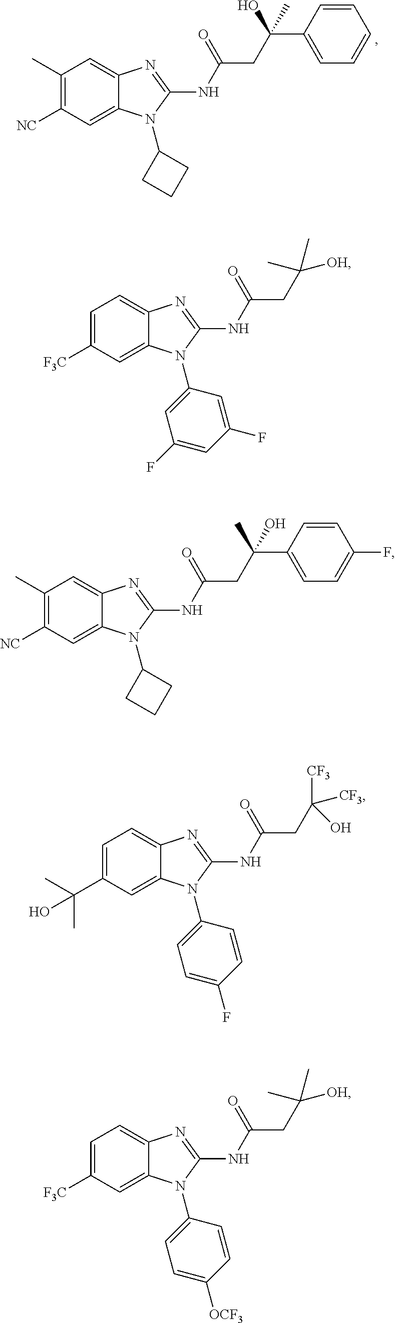 [SMILES: Cc1cc2nc(NC(=O)C[C@@](C)(O)c3ccccc3)n(C3CCC3)c2cc1C#N ; CC(C)(O)CC(=O)Nc1nc2ccc(C(F)(F)F)cc2n1-c1cc(F)cc(F)c1 ; Cc1cc2nc(NC(=O)C[C@](C)(O)c3ccc(F)cc3)n(C3CCC3)c2cc1C#N ; CC(C)(O)c1ccc2nc(NC(=O)CC(O)(C(F)(F)F)C(F)(F)F)n(-c3ccc(F)cc3)c2c1 ; CC(C)(O)CC(=O)Nc1nc2ccc(C(F)(F)F)cc2n1-c1ccc(OC(F)(F)F)cc1]

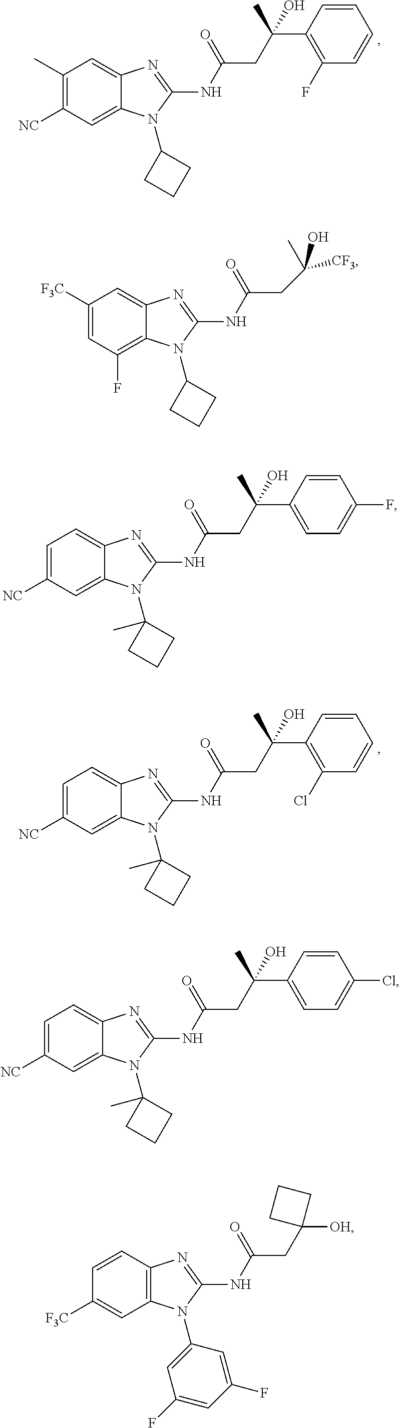 [SMILES: Cc1cc2nc(NC(=O)C[C@](C)(O)c3ccccc3F)n(C3CCC3)c2cc1C#N ; C[C@@](O)(CC(=O)Nc1nc2cc(C(F)(F)F)cc(F)c2n1C1CCC1)C(F)(F)F ; CC1(n2c(NC(=O)C[C@](C)(O)c3ccc(F)cc3)nc3ccc(C#N)cc32)CCC1 ; CC1(n2c(NC(=O)C[C@](C)(O)c3ccccc3Cl)nc3ccc(C#N)cc32)CCC1 ; CC1(n2c(NC(=O)C[C@](C)(O)c3ccc(Cl)cc3)nc3ccc(C#N)cc32)CCC1 ; O=C(CC1(O)CCC1)Nc1nc2ccc(C(F)(F)F)cc2n1-c1cc(F)cc(F)c1]

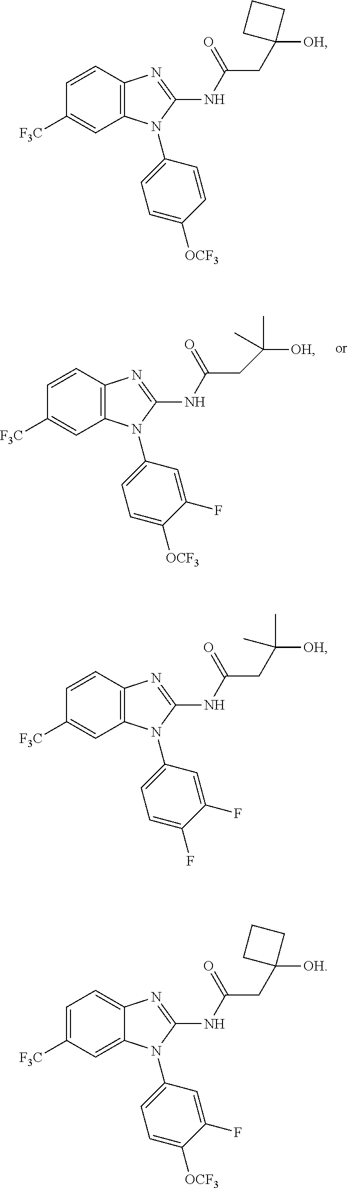 [SMILES: O=C(CC1(O)CCC1)Nc1nc2ccc(C(F)(F)F)cc2n1-c1ccc(OC(F)(F)F)cc1 ; CC(C)(O)CC(=O)Nc1nc2ccc(C(F)(F)F)cc2n1-c1ccc(OC(F)(F)F)c(F)c1 ; "or" ; CC(C)(O)CC(=O)Nc1nc2ccc(C(F)(F)F)cc2n1-c1ccc(F)c(F)c1 ; O=C(CC1(O)CCC1)Nc1nc2ccc(C(F)(F)F)cc2n1-c1ccc(OC(F)(F)F)c(F)c1]

### Formula 2

In an embodiment, the Kv7 channel activator may be selected from one of the following compounds according to Formula 2. Such compounds are described in U.S. Pat. No. 9,481,653 issued Nov. 1, 2016 and corresponding to US Application No. U.S. Ser. No. 14/853,815 filed Sep. 14, 2015; U.S. Pat. No. 9,914,708, issued Mar. 13, 2018 and corresponding to U.S. application Ser. No. 15/339,590 filed October 31; U.S. Pat. No. 10,385,025, issued Aug. 20, 2019 and corresponding to U.S. application Ser. No. 15/879,792 filed Jan. 25, 2018; U.S. Pat. No. 10,906,877 issued on Feb. 2, 2021 and corresponding to U.S. application Ser. No. 16/460,449 filed Jul. 2, 2019; U.S. Pat. No. 10,851,067 issued on Dec. 1, 2020 and corresponding to U.S. application Ser. No. 16/358,642 filed Mar. 19, 2019; U.S. Pat. No. 11,261,162 issued on Mar. 1, 2022 and corresponding to U.S. application Ser. No. 17/077,068 filed Oct. 22, 2020; US Publication No. 20210188782A1, published Jun. 24, 2021 and corresponding to U.S. application Ser. No. 17/127,231 filed Dec. 18, 2020; which are incorporated by reference in their entirety herein. In the case of any conflict of terminology in the context of Formula 2, these references incorporated by reference herein control.

In an embodiment, the Kv7 channel activator is a compound according to formula 2:

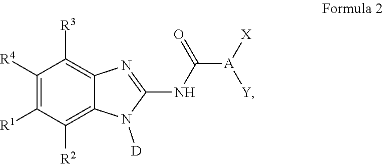

wherein D is optionally substituted cyclobutyl, optionally substituted phenyl, optionally substituted isoxazolyl, optionally substituted pyridinyl, isopropyl, or t-butyl; A is C₂₋₈ alkyl; X is H, F, CF₃ optionally substituted phenyl, or optionally substituted pyridinyl; Y is H, F, Cl, Br, I, or a moiety having a molecular weight of 15 Da to 300 Da and consisting of 2 to 5 chemical elements, wherein the chemical elements are independently C, H, O, N, S, F, Cl, or Br; R¹ is F, Cl, Br, CN, OCH₃, CHF₂, CF₃, C₁₋₄—CO₂-alkyl, C₁₋₄ alkyl, —CH₂CO₂H, —CH₂CO₂CH₂CH₃ or —CH₂CON(CH₃)₂, or C₁₋₅ hydroxyalkyl; and R², R³, and R⁴ are independently H, F, Cl, Br, I, or a substituent having a molecular weight of 15 Da to 200 Da and consisting of 2 to 5 chemical elements, wherein the chemical elements are independently C, H, O, N, S, F, Cl, or Br.

In further embodiments, Y is H, F, CF₃, OH, C₁₋₅ O-alkyl, C₀₋₆ alkylamino, optionally substituted tetrahydropyranyl, or C₀₋₆ fluoroalkylamino.

In further embodiments R¹ is Cl, Br, —OCH₃, —CN, —CF₃, —CH₂OH, —COOCH₂CH₃, —C(CH₃)₂OH, —CHOHCH₂CH₃, —CHOHCH₃, —CHF₂, —CH(CH₃)₂, —C(CH₂CH₃)₂OH, —CH₂COOCH₂CH₃, —CH₂C(CH₃)₂OH, —CH₂COOH, or —CH₂CON(CH₃)₂.

In further embodiments, R² is H, F, —CH₂OH, —CO₂Me, or —C(CH₃)₂OH.

In further embodiments, R³ is H.

In further embodiments, R⁴ is H, —CH₃, or —CF₃.

In further embodiments, R¹ is Cl, Br, CN, OCH₃, CF₃, —CO₂CH₂CH₃, C₁₋₄ alkyl, or C₁₋₄ hydroxyalkyl.

In further embodiments, X is optionally substituted phenyl.

In further embodiments, X is CF₃.

In further embodiments, X is optionally substituted pyridinyl.

In further embodiments, X is H.

In further embodiments, Y is H.

In further embodiments, Y is OH.

In further embodiments, the Kv7 channel activator is a compound selected from the group consisting of:

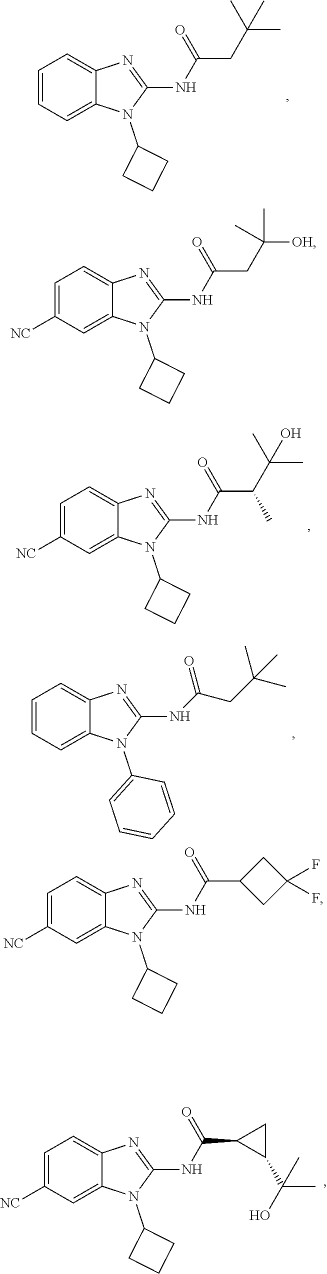

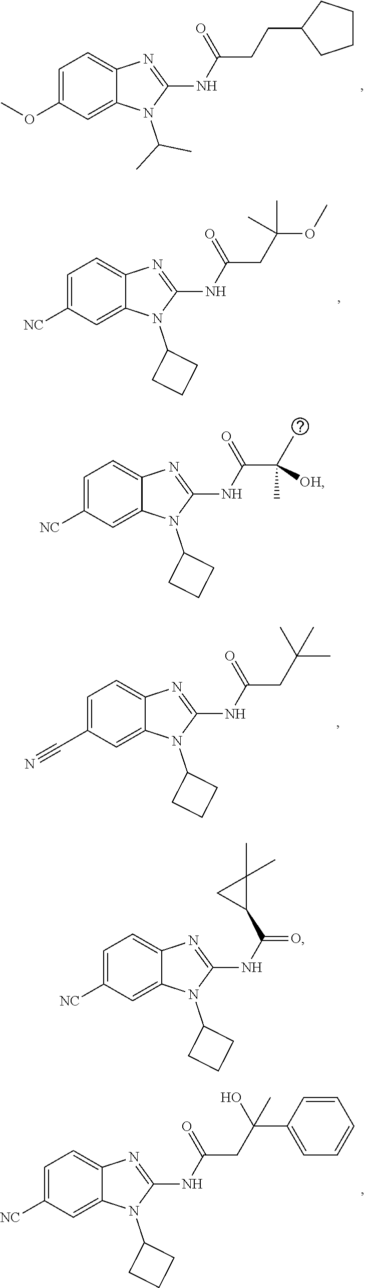

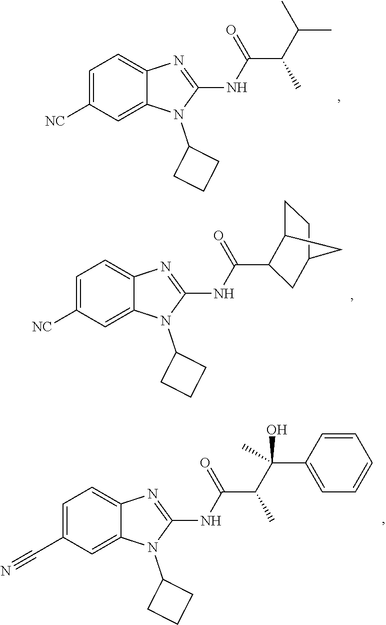 [SMILES: CC(C)[C@H](C)C(=O)Nc1nc2ccc(C#N)cc2n1C1CCC1 ; N#Cc1ccc2nc(NC(=O)C3CC4CCC3C4)n(C3CCC3)c2c1 ; C[C@H](C(=O)Nc1nc2ccc(C#N)cc2n1C1CCC1)[C@@](C)(O)c1ccccc1]

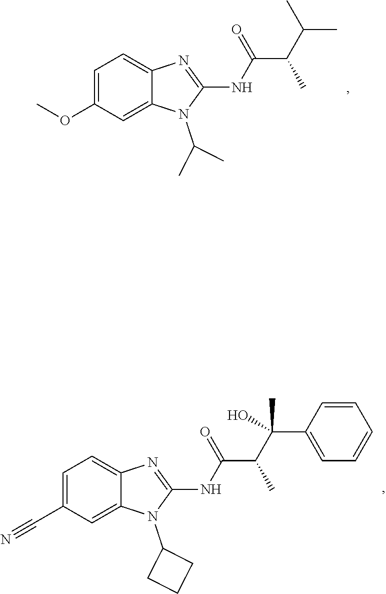 [SMILES: COc1ccc2nc(NC(=O)[C@@H](C)C(C)C)n(C(C)C)c2c1 ; C[C@H](C(=O)Nc1nc2ccc(C#N)cc2n1C1CCC1)[C@](C)(O)c1ccccc1]

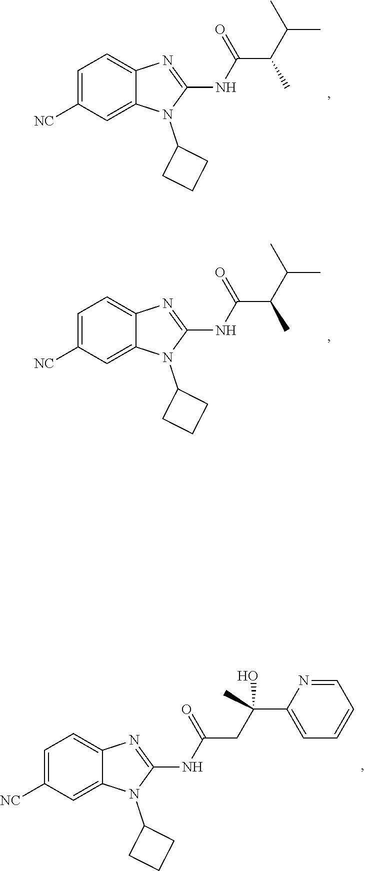 [SMILES: CC(C)[C@H](C)C(=O)Nc1nc2ccc(C#N)cc2n1C1CCC1 ; CC(C)[C@@H](C)C(=O)Nc1nc2ccc(C#N)cc2n1C1CCC1 ; C[C@](O)(CC(=O)Nc1nc2ccc(C#N)cc2n1C1CCC1)c1ccccn1]

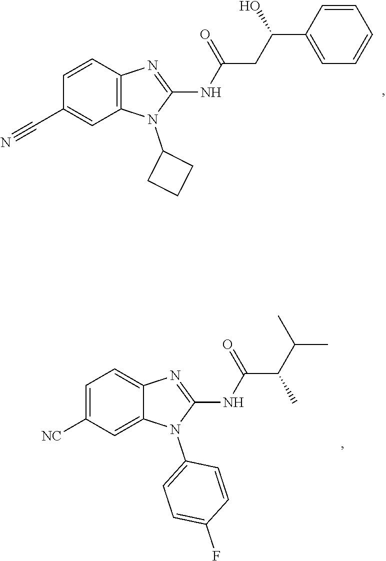 [SMILES: N#Cc1ccc2nc(NC(=O)C[C@H](O)c3ccccc3)n(C3CCC3)c2c1 ; CC(C)[C@H](C)C(=O)Nc1nc2ccc(C#N)cc2n1-c1ccc(F)cc1]

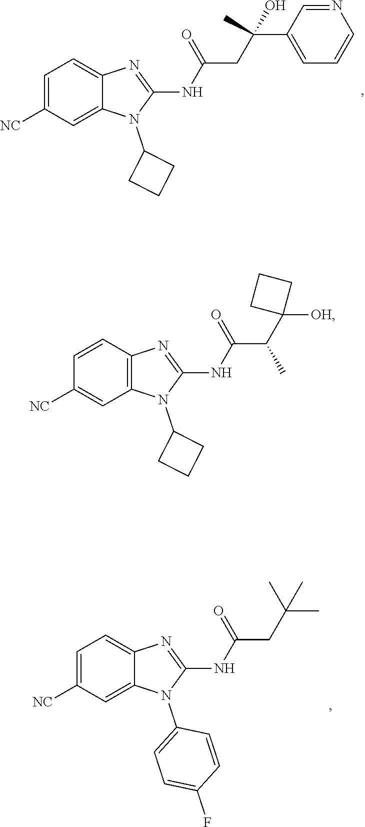 [SMILES: C[C@](O)(CC(=O)Nc1nc2ccc(C#N)cc2n1C1CCC1)c1cccnc1 ; C[C@H](C(=O)Nc1nc2ccc(C#N)cc2n1C1CCC1)C1(O)CCC1 ; CC(C)(C)CC(=O)Nc1nc2ccc(C#N)cc2n1-c1ccc(F)cc1]

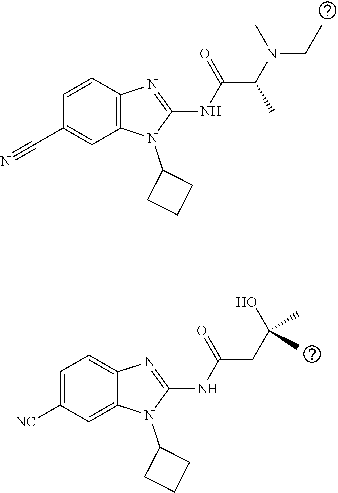

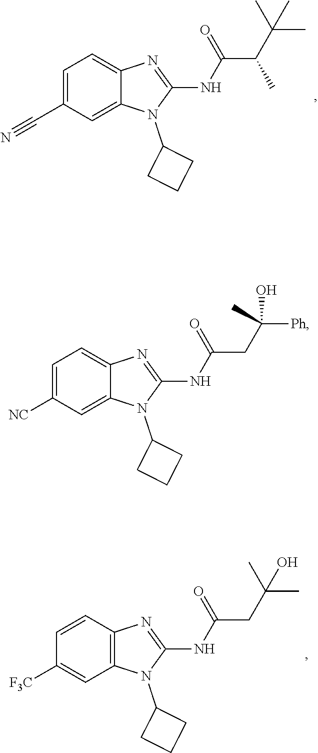 [SMILES: C[C@H](C(=O)Nc1nc2ccc(C#N)cc2n1C1CCC1)C(C)(C)C ; C[C@](O)(CC(=O)Nc1nc2ccc(C#N)cc2n1C1CCC1)c1ccccc1 ; CC(C)(O)CC(=O)Nc1nc2ccc(C(F)(F)F)cc2n1C1CCC1]

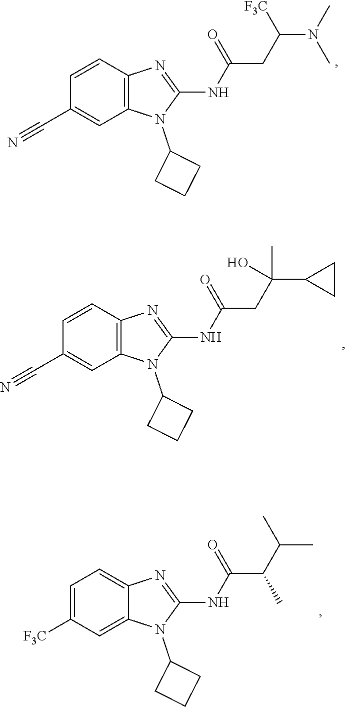 [SMILES: CN(C)C(CC(=O)Nc1nc2ccc(C#N)cc2n1C1CCC1)C(F)(F)F ; CC(O)(CC(=O)Nc1nc2ccc(C#N)cc2n1C1CCC1)C1CC1 ; CC(C)[C@H](C)C(=O)Nc1nc2ccc(C(F)(F)F)cc2n1C1CCC1]

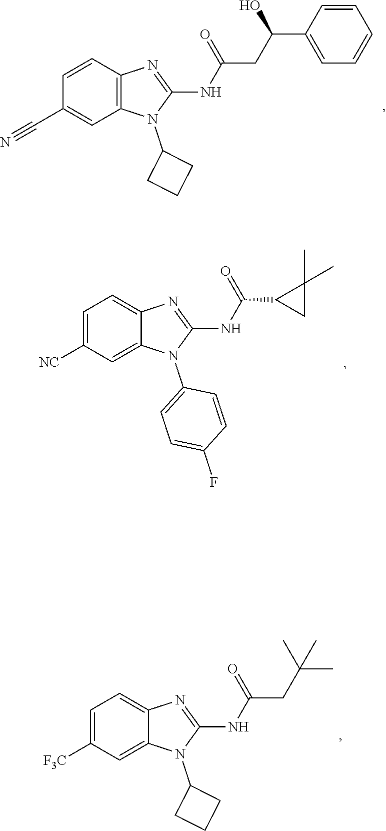

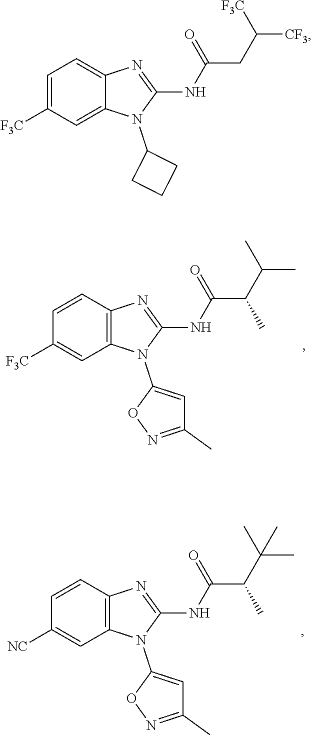 [SMILES: O=C(CC(C(F)(F)F)C(F)(F)F)Nc1nc2ccc(C(F)(F)F)cc2n1C1CCC1 ; Cc1cc(-n2c(NC(=O)[C@@H](C)C(C)C)nc3ccc(C(F)(F)F)cc32)on1 ; Cc1cc(-n2c(NC(=O)[C@@H](C)C(C)(C)C)nc3ccc(C#N)cc32)on1]

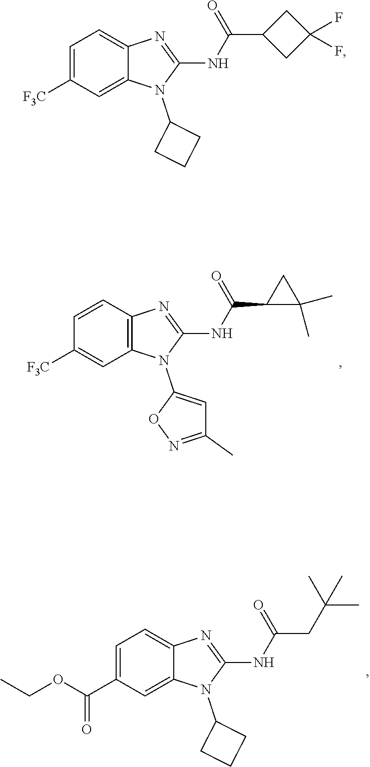

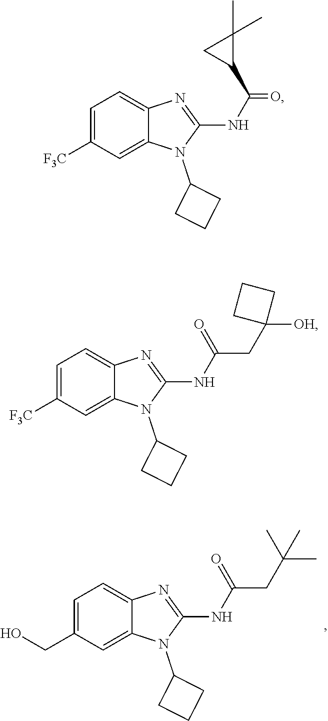

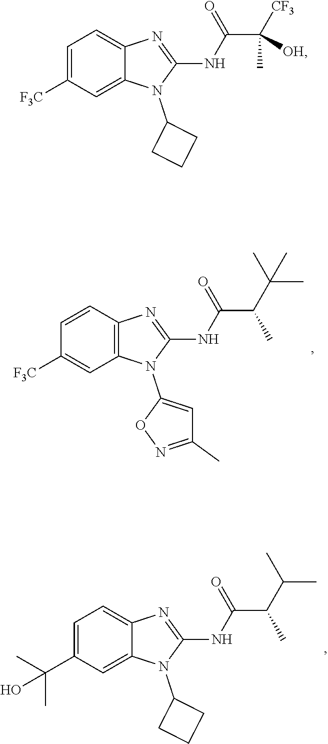

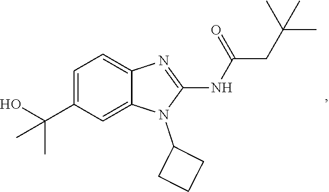 [SMILES: CC(C)(C)CC(=O)Nc1nc2ccc(C(C)(C)O)cc2n1C1CCC1]

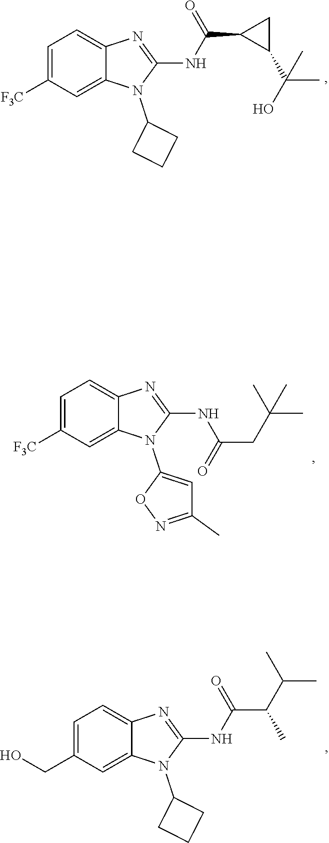 [SMILES: CC(C)(O)[C@H]1C[C@@H]1C(=O)Nc1nc2ccc(C(F)(F)F)cc2n1C1CCC1 ; Cc1cc(-n2c(NC(=O)CC(C)(C)C)nc3ccc(C(F)(F)F)cc32)on1 ; CC(C)[C@H](C)C(=O)Nc1nc2ccc(CO)cc2n1C1CCC1]

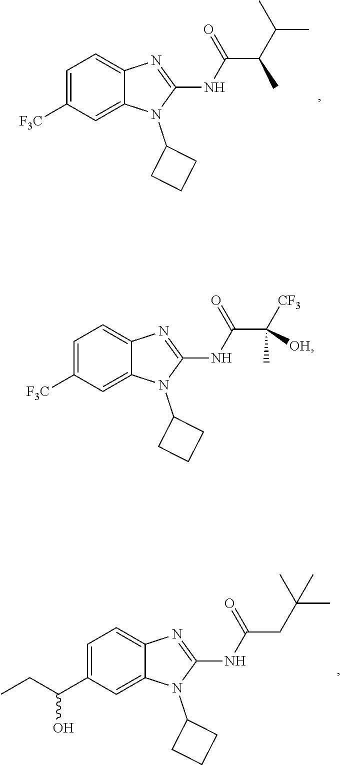

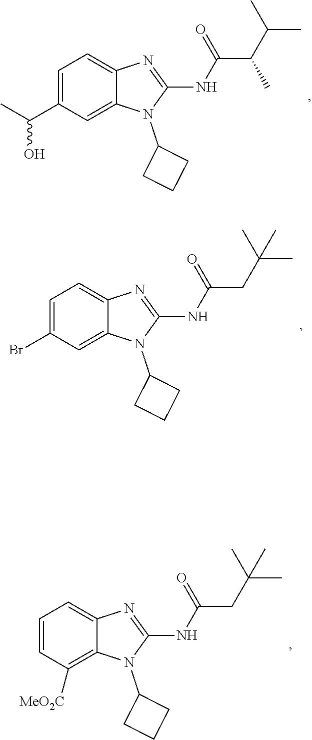 [SMILES: CC(O)c1ccc2nc(NC(=O)[C@@H](C)C(C)C)n(C3CCC3)c2c1 ; CC(C)(C)CC(=O)Nc1nc2ccc(Br)cc2n1C1CCC1 ; COC(=O)c1cccc2nc(NC(=O)CC(C)(C)C)n(C3CCC3)c12]

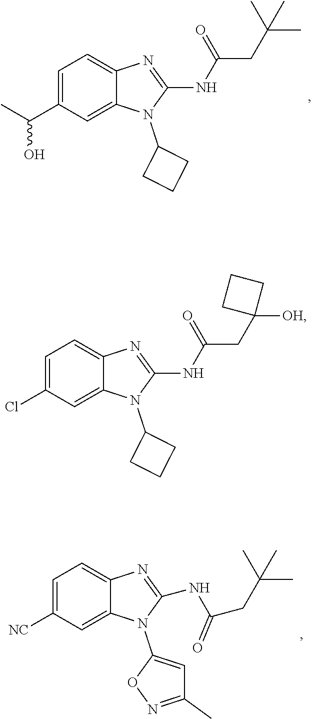 [SMILES: CC(O)c1ccc2nc(NC(=O)CC(C)(C)C)n(C3CCC3)c2c1 ; O=C(CC1(O)CCC1)Nc1nc2ccc(Cl)cc2n1C1CCC1 ; Cc1cc(-n2c(NC(=O)CC(C)(C)C)nc3ccc(C#N)cc32)on1]

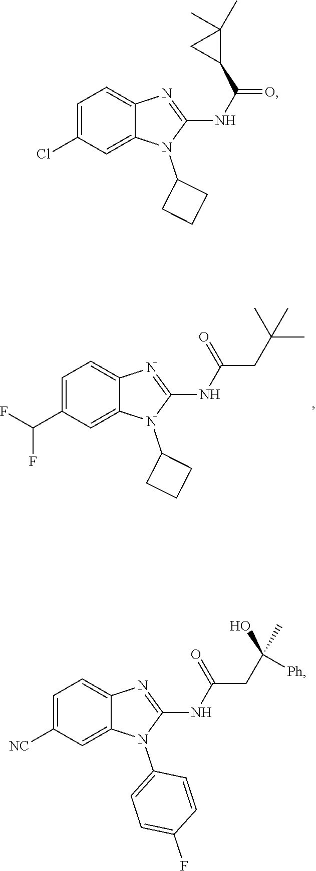 [SMILES: CC1(C)C[C@@H]1C(=O)Nc1nc2ccc(Cl)cc2n1C1CCC1 ; CC(C)(C)CC(=O)Nc1nc2ccc(C(F)F)cc2n1C1CCC1 ; C[C@@](O)(CC(=O)Nc1nc2ccc(C#N)cc2n1-c1ccc(F)cc1)c1ccccc1]

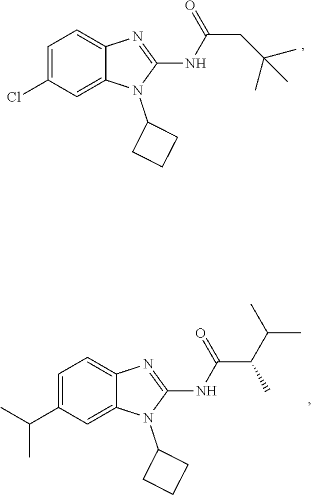

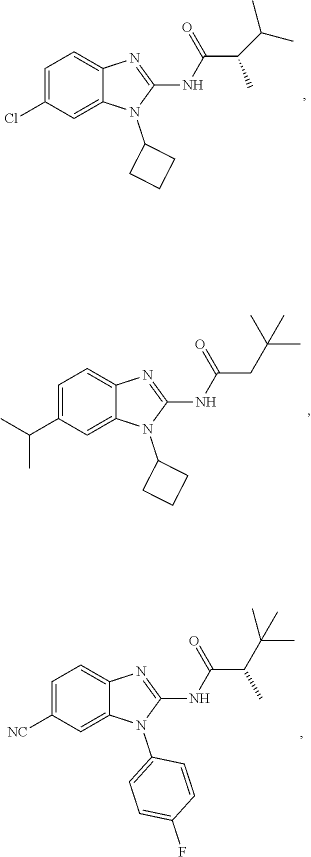

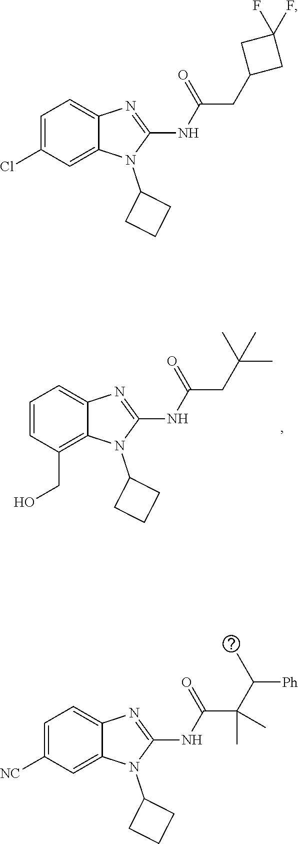 [SMILES: O=C(CC1CC(F)(F)C1)Nc1nc2ccc(Cl)cc2n1C1CCC1 ; CC(C)(C)CC(=O)Nc1nc2cccc(CO)c2n1C1CCC1 ; CC(c1ccccc1)C(C)(C)C(=O)Nc1nc2ccc(C#N)cc2n1C1CCC1]

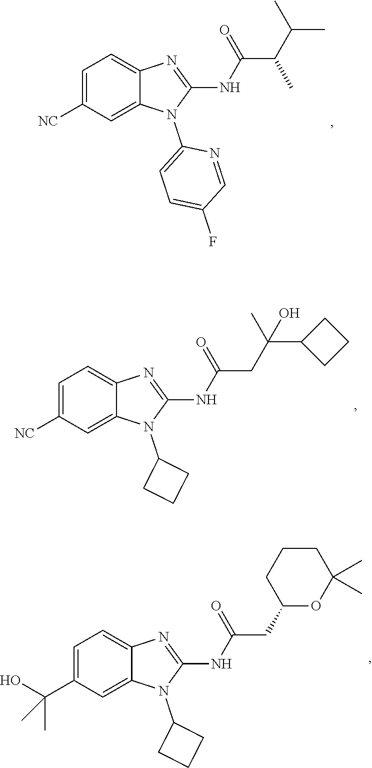 [SMILES: CC(C)[C@H](C)C(=O)Nc1nc2ccc(C#N)cc2n1-c1ccc(F)cn1 ; CC(O)(CC(=O)Nc1nc2ccc(C#N)cc2n1C1CCC1)C1CCC1 ; CC1(C)CCC[C@@H](CC(=O)Nc2nc3ccc(C(C)(C)O)cc3n2C2CCC2)O1]

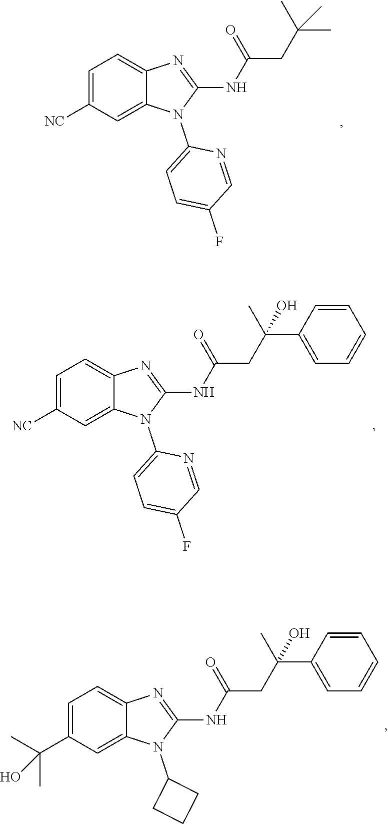

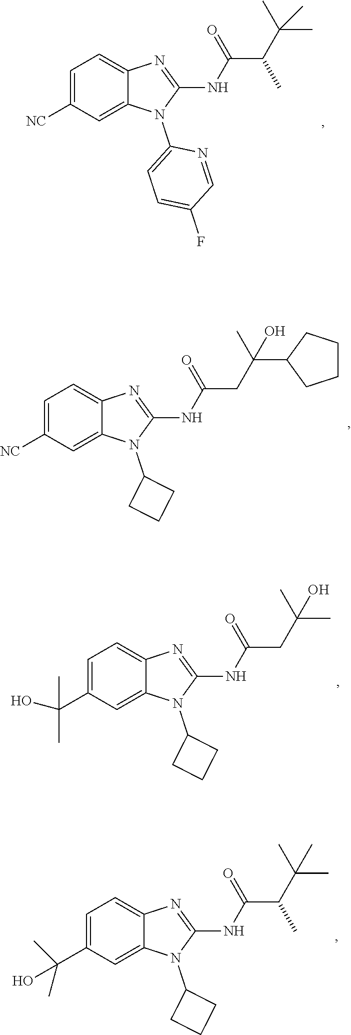 [SMILES: C[C@H](C(=O)Nc1nc2ccc(C#N)cc2n1-c1ccc(F)cn1)C(C)(C)C ; CC(O)(CC(=O)Nc1nc2ccc(C#N)cc2n1C1CCC1)C1CCCC1 ; CC(C)(O)CC(=O)Nc1nc2ccc(C(C)(C)O)cc2n1C1CCC1 ; C[C@H](C(=O)Nc1nc2ccc(C(C)(C)O)cc2n1C1CCC1)C(C)(C)C]

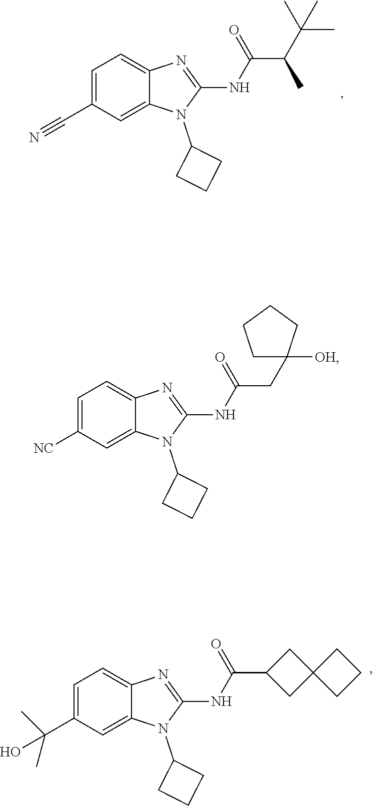 [SMILES: C[C@@H](C(=O)Nc1nc2ccc(C#N)cc2n1C1CCC1)C(C)(C)C ; N#Cc1ccc2nc(NC(=O)CC3(O)CCCC3)n(C3CCC3)c2c1 ; CC(C)(O)c1ccc2nc(NC(=O)C3CC4(CCC4)C3)n(C3CCC3)c2c1]

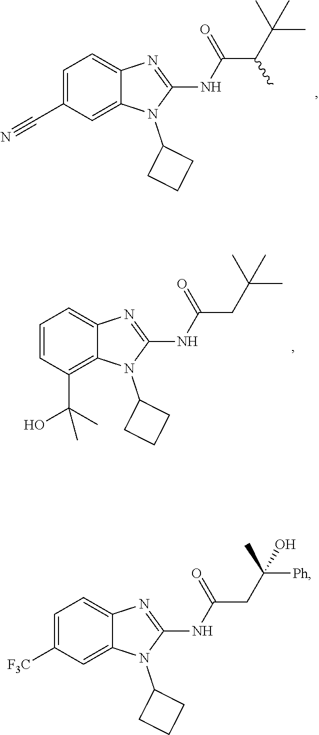 [SMILES: CC(C(=O)Nc1nc2ccc(C#N)cc2n1C1CCC1)C(C)(C)C ; CC(C)(C)CC(=O)Nc1nc2cccc(C(C)(C)O)c2n1C1CCC1 ; C[C@](O)(CC(=O)Nc1nc2ccc(C(F)(F)F)cc2n1C1CCC1)c1ccccc1]

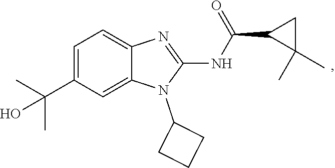

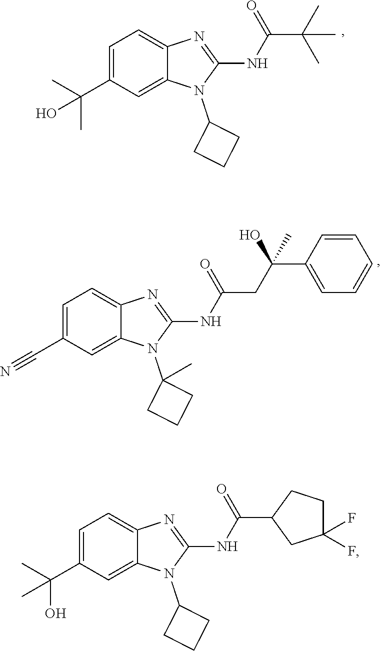

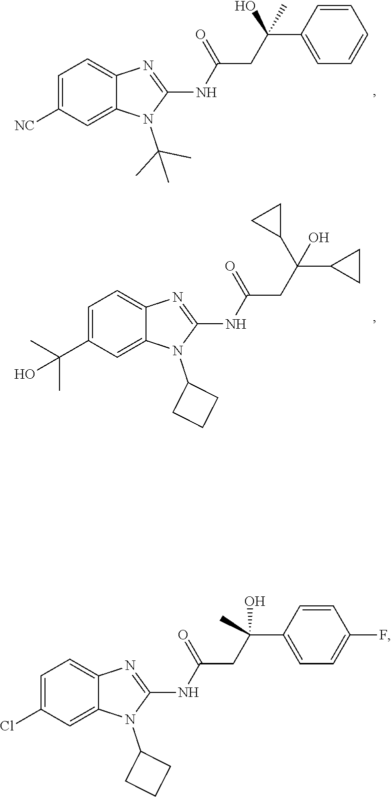 [SMILES: CC(C)(C)n1c(NC(=O)C[C@@](C)(O)c2ccccc2)nc2ccc(C#N)cc21 ; CC(C)(O)c1ccc2nc(NC(=O)CC(O)(C3CC3)C3CC3)n(C3CCC3)c2c1 ; C[C@](O)(CC(=O)Nc1nc2ccc(Cl)cc2n1C1CCC1)c1ccc(F)cc1]

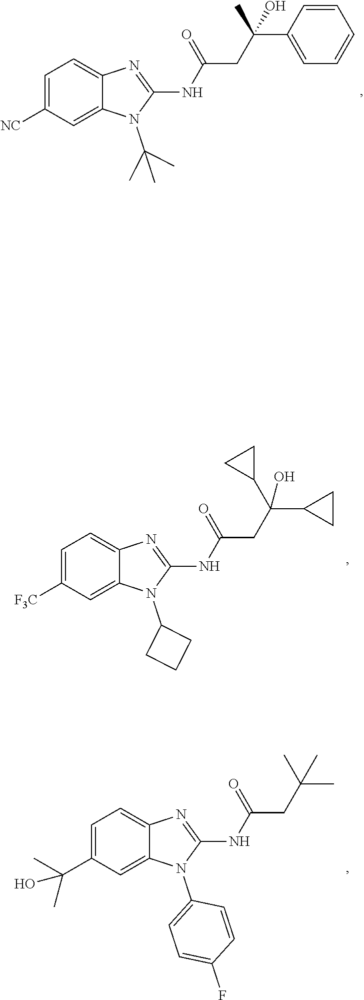 [SMILES: CC(C)(C)n1c(NC(=O)C[C@](C)(O)c2ccccc2)nc2ccc(C#N)cc21 ; O=C(CC(O)(C1CC1)C1CC1)Nc1nc2ccc(C(F)(F)F)cc2n1C1CCC1 ; CC(C)(C)CC(=O)Nc1nc2ccc(C(C)(C)O)cc2n1-c1ccc(F)cc1]

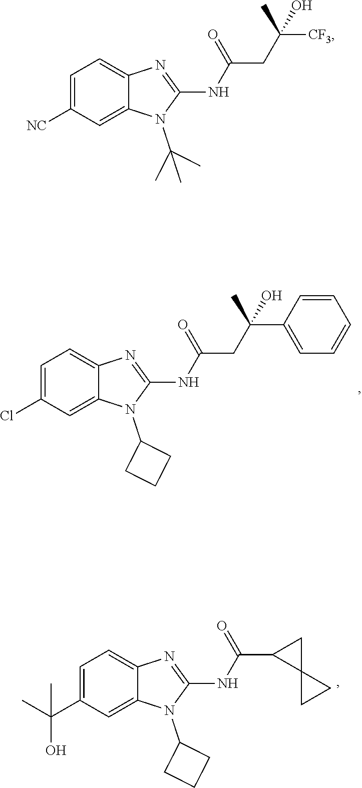 [SMILES: CC(C)(C)n1c(NC(=O)C[C@](C)(O)C(F)(F)F)nc2ccc(C#N)cc21 ; C[C@](O)(CC(=O)Nc1nc2ccc(Cl)cc2n1C1CCC1)c1ccccc1 ; CC(C)(O)c1ccc2nc(NC(=O)C3CC34CC4)n(C3CCC3)c2c1]

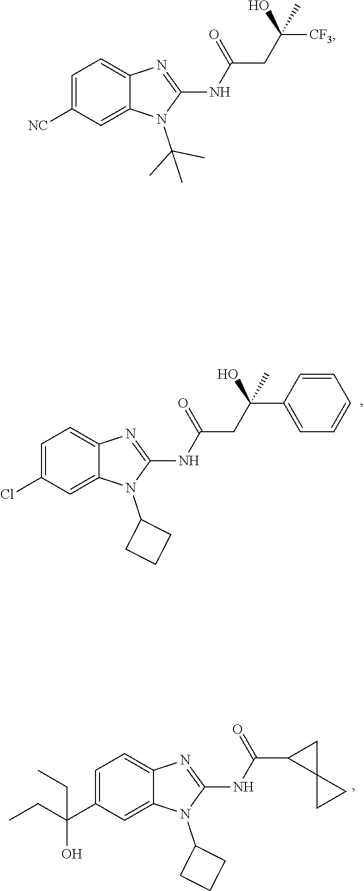 [SMILES: CC(C)(C)n1c(NC(=O)C[C@@](C)(O)C(F)(F)F)nc2ccc(C#N)cc21 ; C[C@@](O)(CC(=O)Nc1nc2ccc(Cl)cc2n1C1CCC1)c1ccccc1 ; CCC(O)(CC)c1ccc2nc(NC(=O)C3CC34CC4)n(C3CCC3)c2c1]

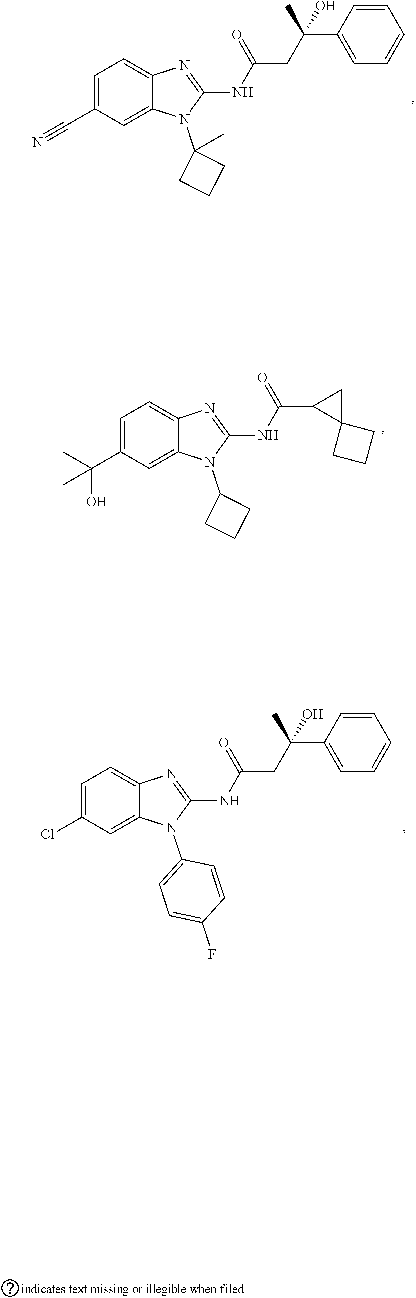 [SMILES: CC1(n2c(NC(=O)C[C@](C)(O)c3ccccc3)nc3ccc(C#N)cc32)CCC1 ; CC(C)(O)c1ccc2nc(NC(=O)C3CC34CCC4)n(C3CCC3)c2c1 ; C[C@](O)(CC(=O)Nc1nc2ccc(Cl)cc2n1-c1ccc(F)cc1)c1ccccc1]

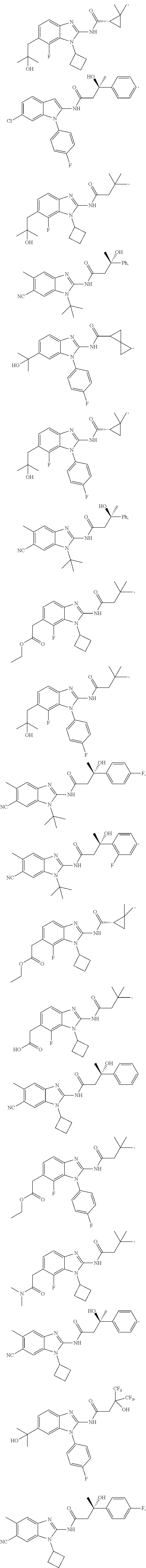

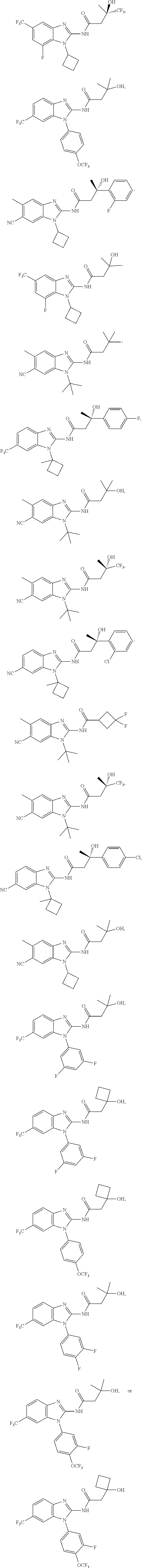

In further embodiments, the Kv7 channel activator is a compound according to formula 2 wherein, D is optionally substituted cyclobutyl, or t-butyl; A is C₂₋₈ alkyl; X is H, CF₃, or optionally substituted phenyl; Y is H or OH; R¹ is CN or C₁₋₄ hydroxyalkyl; and R², R³, and R⁴ are independently H, or F.

In further embodiments, R¹ is CN, —C(CH₃)₂OH, or —CH₂C(CH₃)₂OH.

In further embodiments, R² is F.

In further embodiments, R³ is H.

In further embodiments, R⁴ is H.

In further embodiments, R¹ is CN.

In further embodiments, R¹ is C₁₋₄ hydroxyalkyl.

In further embodiments, X is optionally substituted phenyl.

In further embodiments, X is CF₃.

In further embodiments, X is H.

In further embodiments, Y is H.

In further embodiments, Y is OH.

In further embodiments, the Kv7 channel activator is a compound according to formula 2 wherein, D is cyclobutyl; A is C₁₋₈ alkyl; X is CF₃; Y is H; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃; or
D is optionally substituted cyclobutyl; A is C₁₋₈ alkyl; X is CF₃; Y is H; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃; or
D is t-butyl; A is C₁₋₈ alkyl; X is H; Y is H; R¹ is Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C (CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃; or
D is t-butyl; A is C₁₋₈ alkyl; X is CF₃; Y is H; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C (CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃; or
D is cyclobutyl; A is C₁₋₈ alkyl; X is H; Y is methyl (2,2,2-trifluoroethyl)amino; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃; or
D is cyclobutyl; A is C₁₋₈ alkyl; X is CF₃; Y is dimethylamino; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃; or
D is optionally substituted cyclobutyl, optionally substituted phenyl, or optionally substituted C₂₋₅ alkyl, wherein the optional substituents are selected from —CH₃ and F; A is C₁ alkyl; X is substituted cyclobutyl, wherein the substituent is F; Y is H; R¹ is selected from H, C3 hydroxyalkyl, CN, F, or Cl; R² is selected from H, CN, F, Br, or —OCF₃; R³ is selected from H, F, or —OCH₃; R⁴ is H or F; and wherein when X is substituted with 2 fluorine atoms, the fluorine atoms are not geminal; or a pharmaceutically acceptable salt thereof;

In an embodiment, the Kv7 channel activator is a compound according to formula 2. Wherein, R¹, R², R³, and R⁴ are independently H, F, Cl, Br, I, or a substituent having a molecular weight of 15 Da to 200 Da and consisting of 2 to 5 chemical elements, wherein the chemical elements are independently C, H, O, N, S, F, Cl, or Br.

In further embodiments, Y is H, F, CF₃, OH, C₁₋₅ O-alkyl, C₀₋₆ alkylamino, optionally substituted tetrahydropyranyl, or C₀₋₆ fluoroalkylamino.

In further embodiments, R¹ is H, Cl, Br, —OCH₃, —CN, —CF₃, —CH₂OH, —COOCH₂CH₃, —C(CH₃)₂OH, —CHOHCH₂CH₃, —CHOHCH₃, —CHF₂, —CH(CH₃)₂, —C (CH₂CH₃)OH, —CH₂COOCH₂CH₃, —CH₂C(CH₃)₂OH, —CH₂COOH, or —CH₂CON(CH₃)₂.

In further embodiments, R² is H, F, —CH₂OH, —CO₂Me, or —C(CH₃)₂OH.

In further embodiments, R³ is H.

In further embodiments, R⁴ is H, —CH₃, or —CF₃.

In further embodiments, the Kv7 channel activator is a compound selected from the group consisting of:

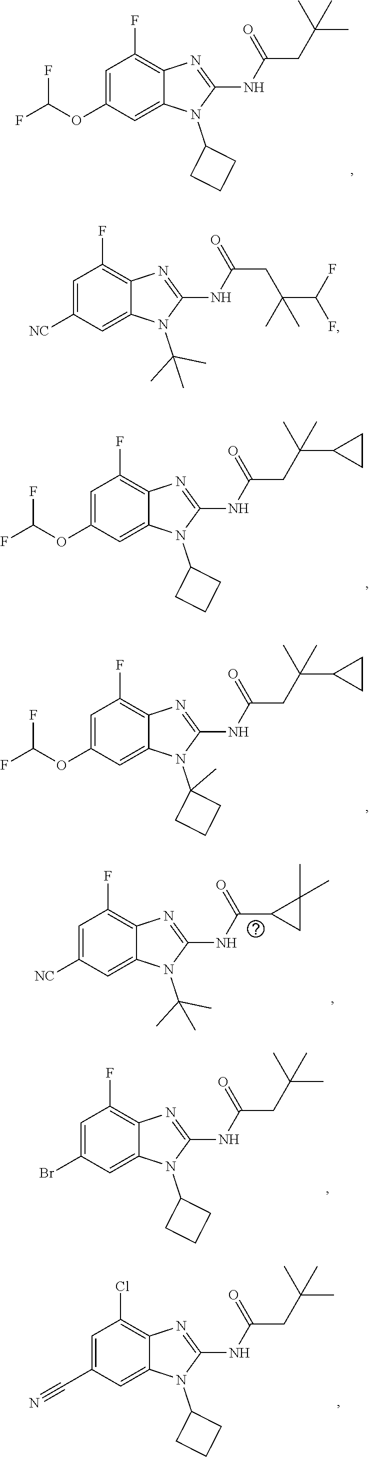

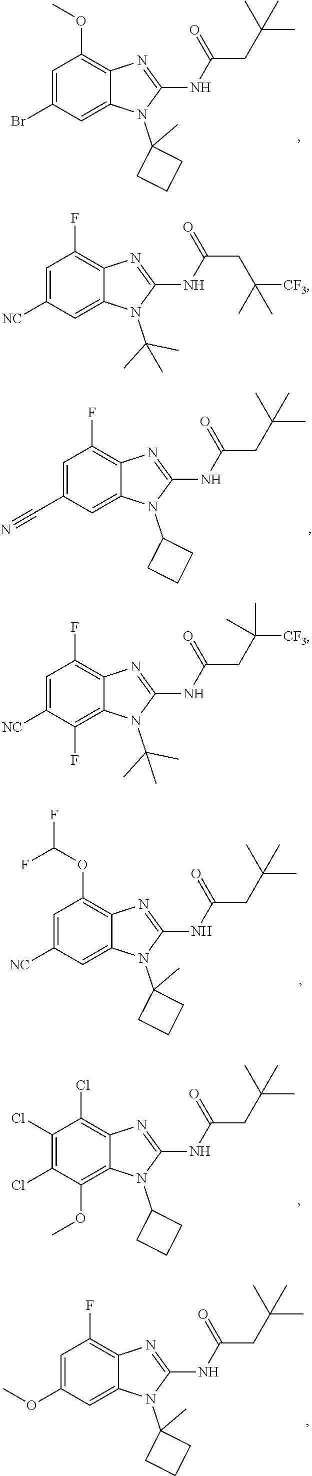

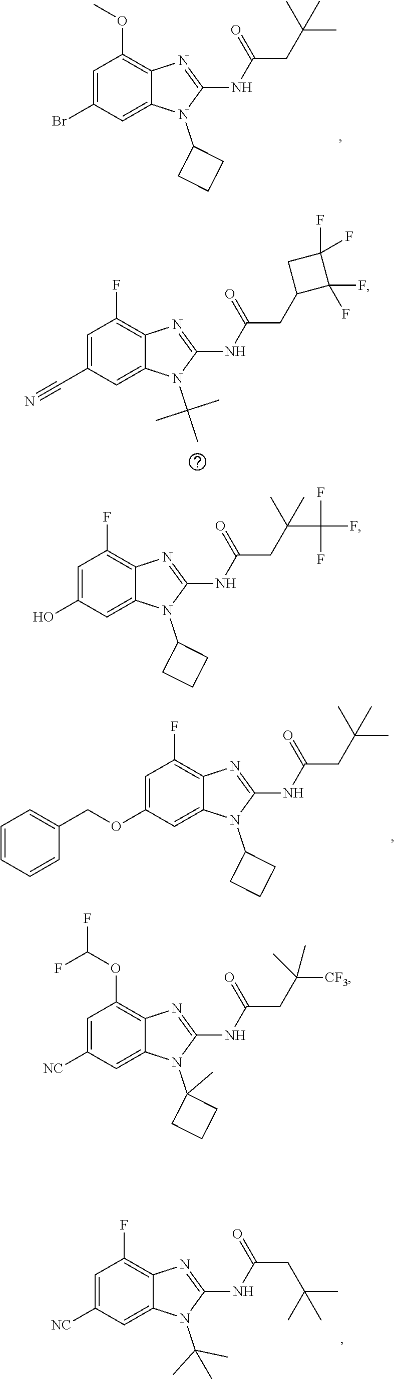

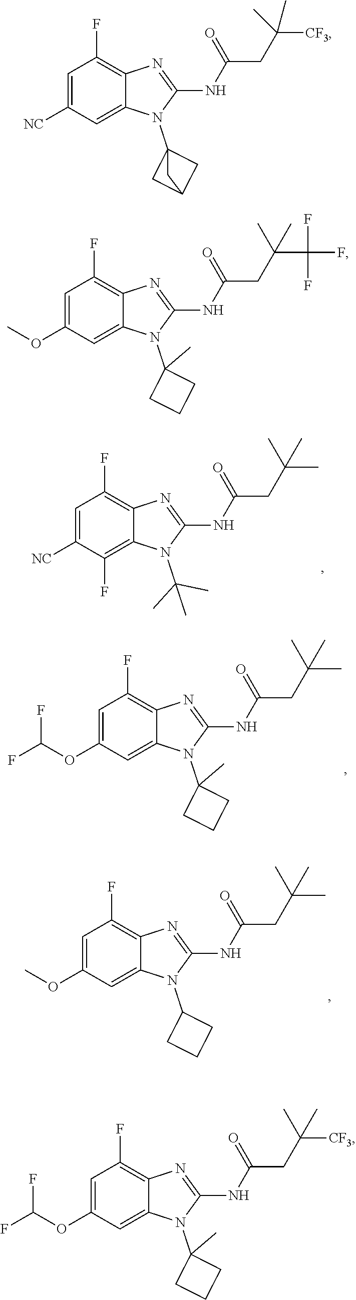

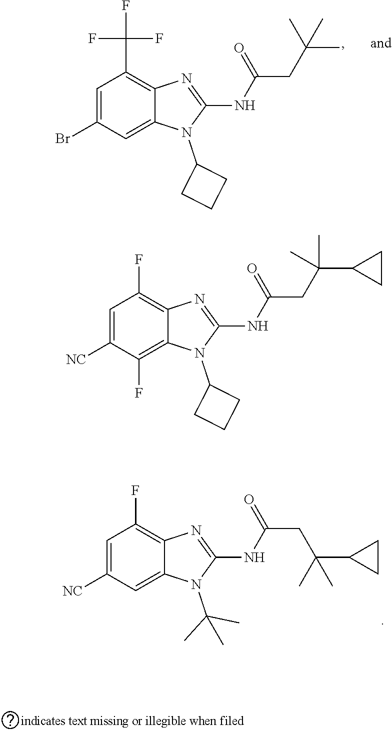 [SMILES: CC(C)(C)CC(=O)Nc1nc2c(C(F)(F)F)cc(Br)cc2n1C1CCC1 ; CC(C)(CC(=O)Nc1nc2c(F)cc(C#N)c(F)c2n1C1CCC1)C1CC1 ; CC(C)(CC(=O)Nc1nc2c(F)cc(C#N)cc2n1C(C)(C)C)C1CC1]

or a pharmaceutically acceptable salt thereof.

In further embodiments, the Kv7 channel activator is a compound selected from the group consisting of:

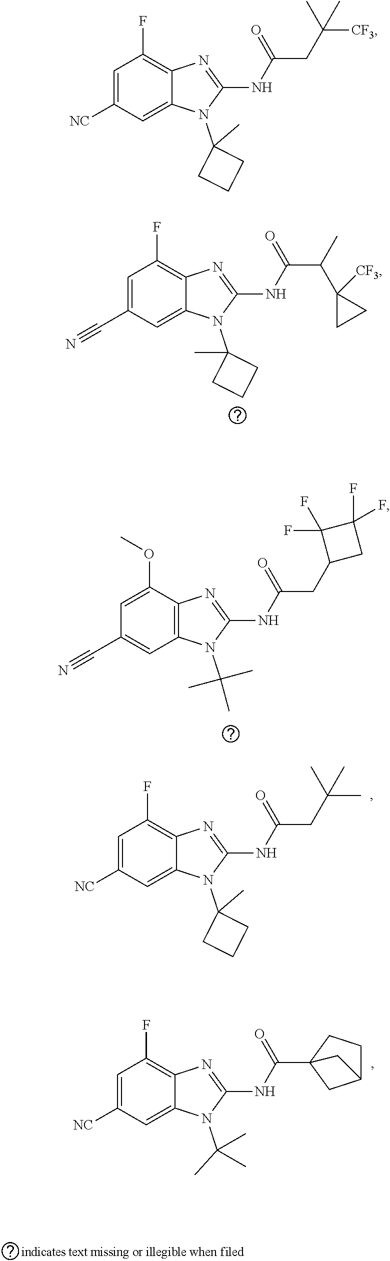 [SMILES: CC1(n2c(NC(=O)CC(C)(C)C(F)(F)F)nc3c(F)cc(C#N)cc32)CCC1 ; CC(C(=O)Nc1nc2c(F)cc(C#N)cc2n1C1(C)CCC1)C1(C(F)(F)F)CC1 ; COc1cc(C#N)cc2c1nc(NC(=O)CC1CC(F)(F)C1(F)F)n2C(C)(C)C ; CC(C)(C)CC(=O)Nc1nc2c(F)cc(C#N)cc2n1C1(C)CCC1 ; CC(C)(C)n1c(NC(=O)C23CCC(C2)C3)nc2c(F)cc(C#N)cc21]

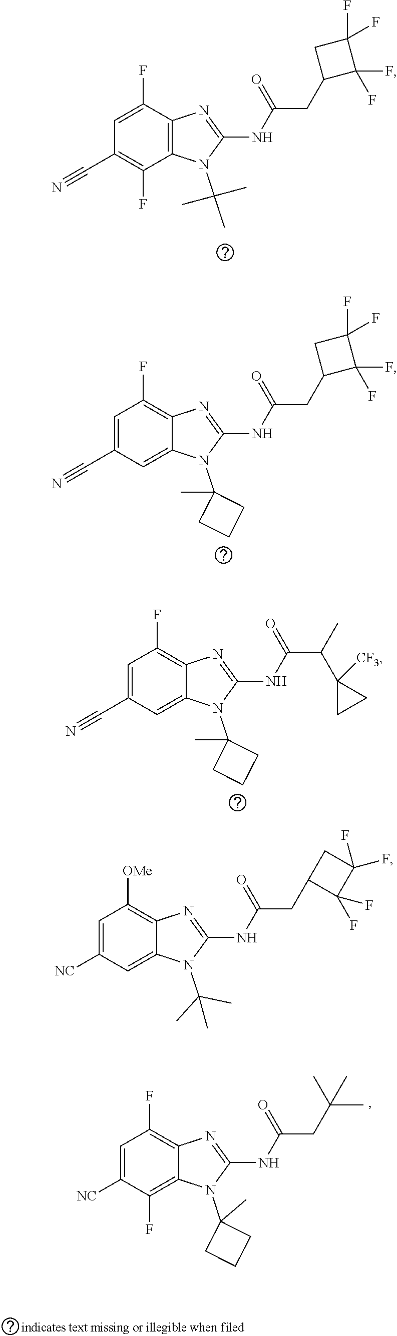 [SMILES: CC(C)(C)n1c(NC(=O)CC2CC(F)(F)C2(F)F)nc2c(F)cc(C#N)c(F)c21 ; CC1(n2c(NC(=O)CC3CC(F)(F)C3(F)F)nc3c(F)cc(C#N)cc32)CCC1 ; CC(C(=O)Nc1nc2c(F)cc(C#N)cc2n1C1(C)CCC1)C1(C(F)(F)F)CC1 ; COc1cc(C#N)cc2c1nc(NC(=O)CC1CC(F)(F)C1(F)F)n2C(C)(C)C ; CC(C)(C)CC(=O)Nc1nc2c(F)cc(C#N)c(F)c2n1C1(C)CCC1]

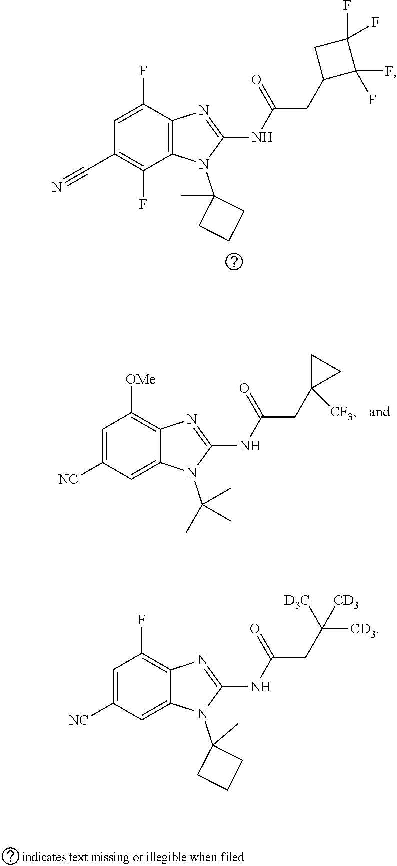 [SMILES: CC1(n2c(NC(=O)CC3CC(F)(F)C3(F)F)nc3c(F)cc(C#N)c(F)c32)CCC1 ; COc1cc(C#N)cc2c1nc(NC(=O)CC1(C(F)(F)F)CC1)n2C(C)(C)C ; [2H]C([2H])([2H])C(CC(=O)Nc1nc2c(F)cc(C#N)cc2n1C1(C)CCC1)(C([2H])([2H])[2H])C([2H])([2H])[2H]]

or a pharmaceutically acceptable salt thereof.

In further embodiments the Kv7 channel activator is a compound selected from the group consisting of:

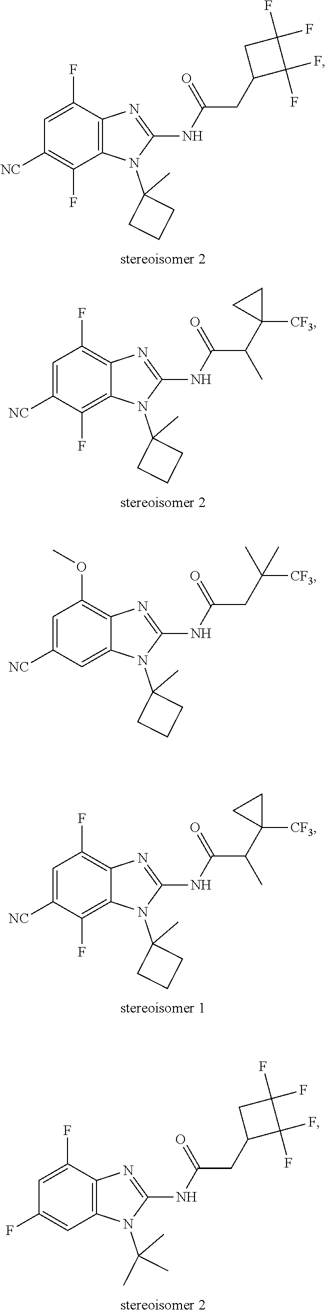 [SMILES: CC1(n2c(NC(=O)CC3CC(F)(F)C3(F)F)nc3c(F)cc(C#N)c(F)c32)CCC1 ; CC(C(=O)Nc1nc2c(F)cc(C#N)c(F)c2n1C1(C)CCC1)C1(C(F)(F)F)CC1 ; COc1cc(C#N)cc2c1nc(NC(=O)CC(C)(C)C(F)(F)F)n2C1(C)CCC1 ; CC(C(=O)Nc1nc2c(F)cc(C#N)c(F)c2n1C1(C)CCC1)C1(C(F)(F)F)CC1 ; CC(C)(C)n1c(NC(=O)CC2CC(F)(F)C2(F)F)nc2c(F)cc(F)cc21]

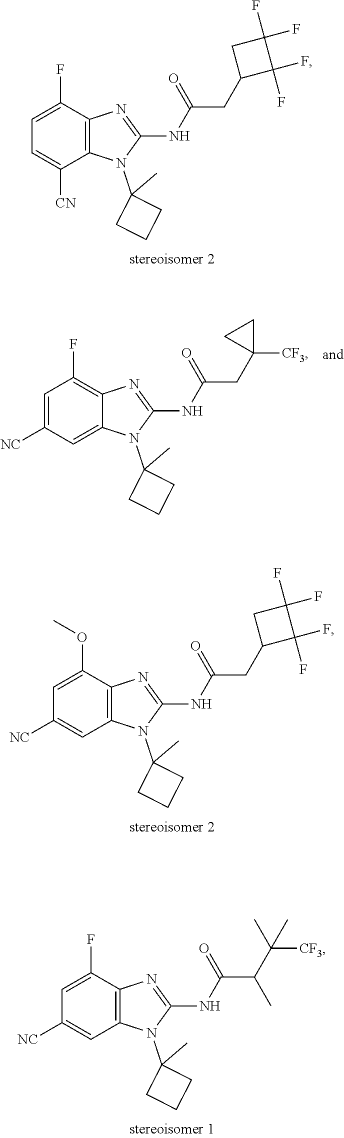 [SMILES: CC1(n2c(NC(=O)CC3CC(F)(F)C3(F)F)nc3c(F)ccc(C#N)c32)CCC1 ; CC1(n2c(NC(=O)CC3(C(F)(F)F)CC3)nc3c(F)cc(C#N)cc32)CCC1 ; COc1cc(C#N)cc2c1nc(NC(=O)CC1CC(F)(F)C1(F)F)n2C1(C)CCC1 ; CC(C(=O)Nc1nc2c(F)cc(C#N)cc2n1C1(C)CCC1)C(C)(C)C(F)(F)F]

or a pharmaceutically acceptable salt thereof.

In further embodiments, the Kv7 channel activator is a compound selected from the group consisting of:

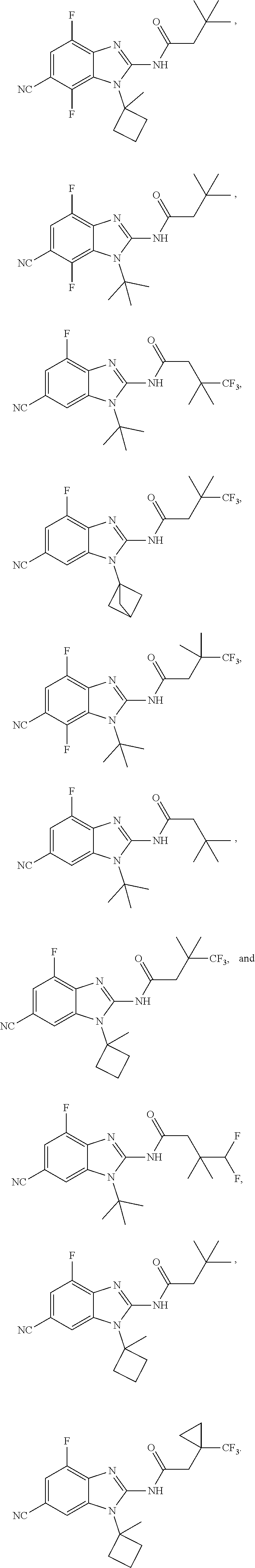

or a pharmaceutically acceptable salt thereof.

In further embodiments, the compound is:

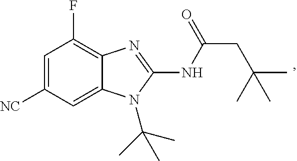 [SMILES: CC(C)(C)CC(=O)Nc1nc2c(F)cc(C#N)cc2n1C(C)(C)C]

or a pharmaceutically acceptable salt thereof.

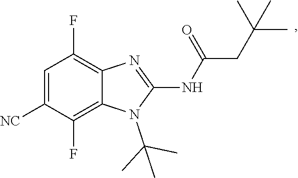 [SMILES: CC(C)(C)CC(=O)Nc1nc2c(F)cc(C#N)c(F)c2n1C(C)(C)C]

or a pharmaceutically acceptable salt thereof.

In further embodiments, the compound is:

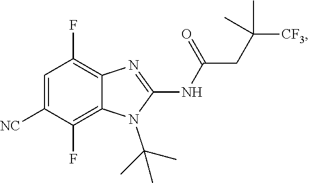 [SMILES: CC(C)(C)n1c(NC(=O)CC(C)(C)C(F)(F)F)nc2c(F)cc(C#N)c(F)c21]

or a pharmaceutically acceptable salt thereof.

In further embodiments, the compound is:

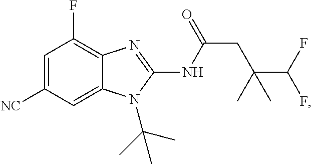 [SMILES: CC(C)(CC(=O)Nc1nc2c(F)cc(C#N)cc2n1C(C)(C)C)C(F)F]

or a pharmaceutically acceptable salt thereof.

In further embodiments, the compound is:

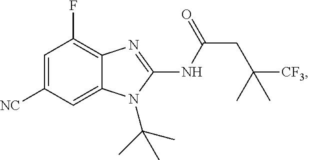 [SMILES: CC(C)(C)n1c(NC(=O)CC(C)(C)C(F)(F)F)nc2c(F)cc(C#N)cc21]

or a pharmaceutically acceptable salt thereof.

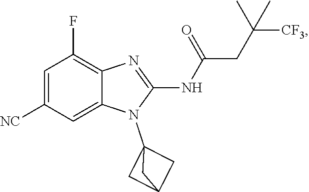 [SMILES: CC(C)(CC(=O)Nc1nc2c(F)cc(C#N)cc2n1C12CC(C1)C2)C(F)(F)F]

or a pharmaceutically acceptable salt thereof.

In further embodiments, the compound is:

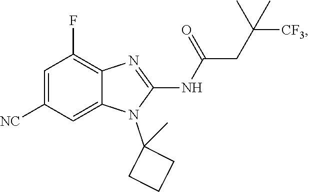 [SMILES: CC1(n2c(NC(=O)CC(C)(C)C(F)(F)F)nc3c(F)cc(C#N)cc32)CCC1]

or a pharmaceutically acceptable salt thereof.

In further embodiments, the compound is:

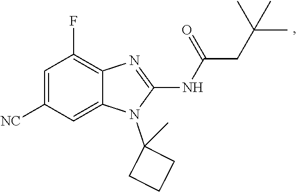 [SMILES: CC(C)(C)CC(=O)Nc1nc2c(F)cc(C#N)cc2n1C1(C)CCC1]

or a pharmaceutically acceptable salt thereof.

In further embodiments, the compound is:

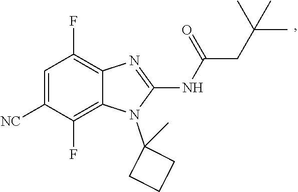 [SMILES: CC(C)(C)CC(=O)Nc1nc2c(F)cc(C#N)c(F)c2n1C1(C)CCC1]

or a pharmaceutically acceptable salt thereof.

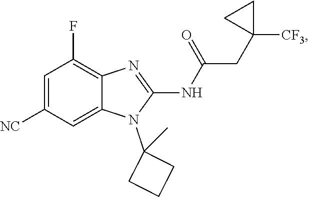 [SMILES: CC1(n2c(NC(=O)CC3(C(F)(F)F)CC3)nc3c(F)cc(C#N)cc32)CCC1]

or a pharmaceutically acceptable salt thereof.

In further embodiments, the Kv7 channel activator is a compound selected from the group consisting of:

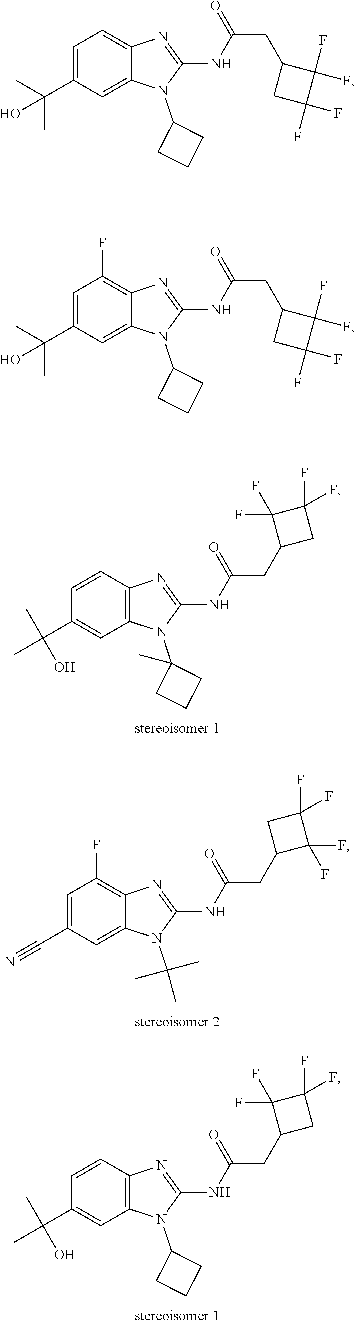 [SMILES: CC(C)(O)c1ccc2nc(NC(=O)CC3CC(F)(F)C3(F)F)n(C3CCC3)c2c1 ; CC(C)(O)c1cc(F)c2nc(NC(=O)CC3CC(F)(F)C3(F)F)n(C3CCC3)c2c1 ; CC(C)(O)c1ccc2nc(NC(=O)CC3CC(F)(F)C3(F)F)n(C3(C)CCC3)c2c1 ; CC(C)(C)n1c(NC(=O)CC2CC(F)(F)C2(F)F)nc2c(F)cc(C#N)cc21 ; CC(C)(O)c1ccc2nc(NC(=O)CC3CC(F)(F)C3(F)F)n(C3CCC3)c2c1]

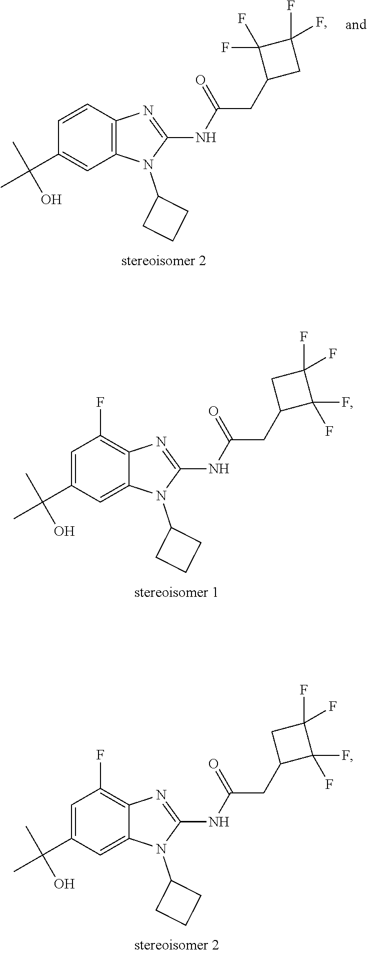 [SMILES: CC(C)(O)c1ccc2nc(NC(=O)CC3CC(F)(F)C3(F)F)n(C3CCC3)c2c1 ; CC(C)(O)c1cc(F)c2nc(NC(=O)CC3CC(F)(F)C3(F)F)n(C3CCC3)c2c1 ; CC(C)(O)c1cc(F)c2nc(NC(=O)CC3CC(F)(F)C3(F)F)n(C3CCC3)c2c1]

or a pharmaceutically acceptable salt thereof.

In further embodiments, the Kv7 channel activator is a compound selected from the group consisting of:

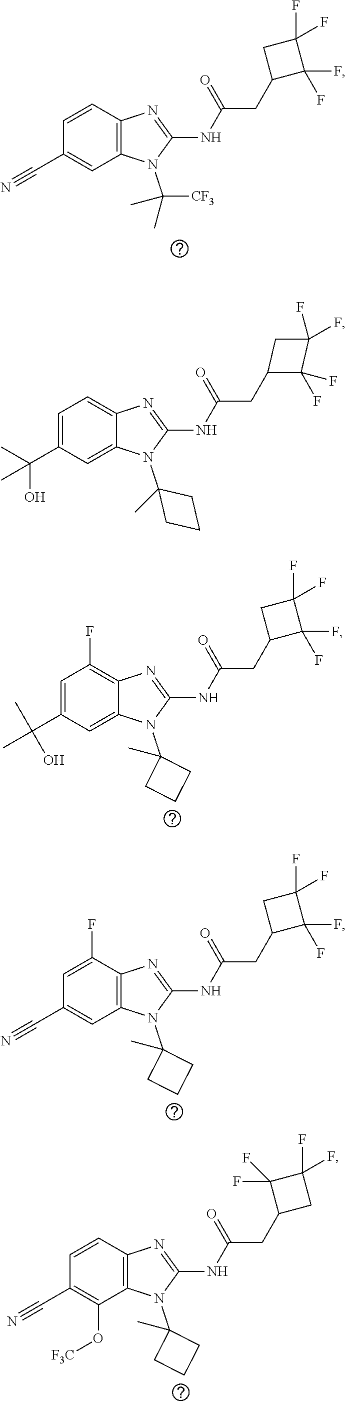 [SMILES: CC(C)(n1c(NC(=O)CC2CC(F)(F)C2(F)F)nc2ccc(C#N)cc21)C(F)(F)F ; CC(C)(O)c1ccc2nc(NC(=O)CC3CC(F)(F)C3(F)F)n(C3(C)CCC3)c2c1 ; CC(C)(O)c1cc(F)c2nc(NC(=O)CC3CC(F)(F)C3(F)F)n(C3(C)CCC3)c2c1 ; CC1(n2c(NC(=O)CC3CC(F)(F)C3(F)F)nc3c(F)cc(C#N)cc32)CCC1 ; CC1(n2c(NC(=O)CC3CC(F)(F)C3(F)F)nc3ccc(C#N)c(OC(F)(F)F)c32)CCC1]

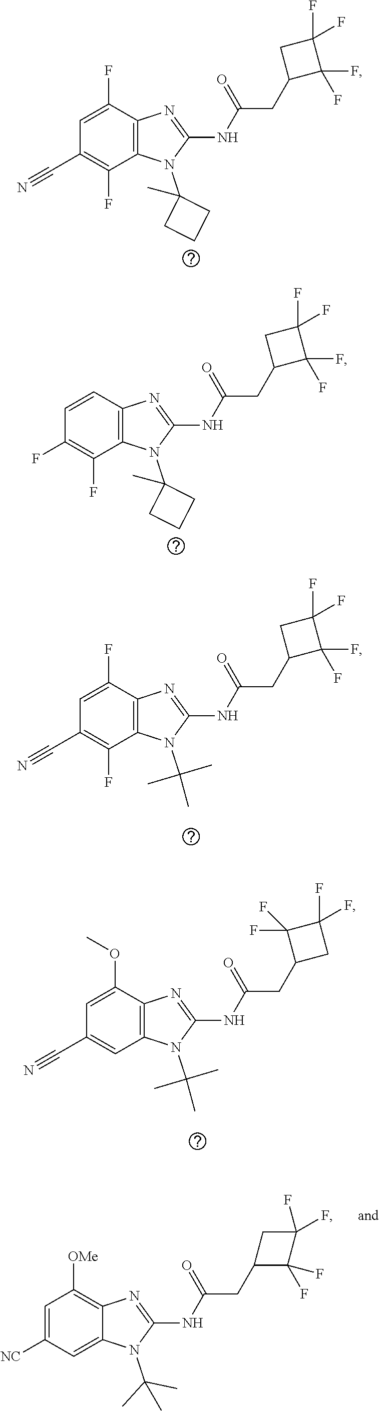 [SMILES: CC1(n2c(NC(=O)CC3CC(F)(F)C3(F)F)nc3c(F)cc(C#N)c(F)c32)CCC1 ; CC1(n2c(NC(=O)CC3CC(F)(F)C3(F)F)nc3ccc(F)c(F)c32)CCC1 ; CC(C)(C)n1c(NC(=O)CC2CC(F)(F)C2(F)F)nc2c(F)cc(C#N)c(F)c21 ; COc1cc(C#N)cc2c1nc(NC(=O)CC1CC(F)(F)C1(F)F)n2C(C)(C)C ; COc1cc(C#N)cc2c1nc(NC(=O)CC1CC(F)(F)C1(F)F)n2C(C)(C)C ; "and"]

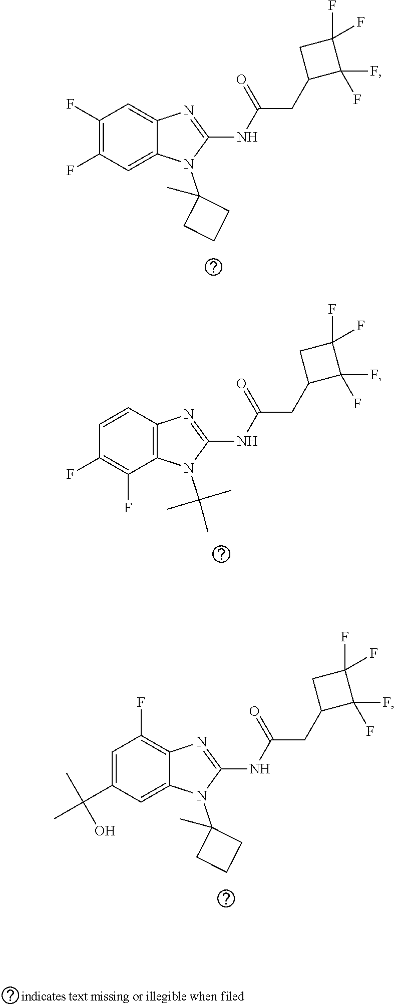 [SMILES: CC1(n2c(NC(=O)CC3CC(F)(F)C3(F)F)nc3cc(F)c(F)cc32)CCC1 ; CC(C)(C)n1c(NC(=O)CC2CC(F)(F)C2(F)F)nc2ccc(F)c(F)c21 ; CC(C)(O)c1cc(F)c2nc(NC(=O)CC3CC(F)(F)C3(F)F)n(C3(C)CCC3)c2c1]

or a pharmaceutically acceptable salt thereof.

In further embodiments, the Kv7 channel activator is a compound selected from the group consisting of:

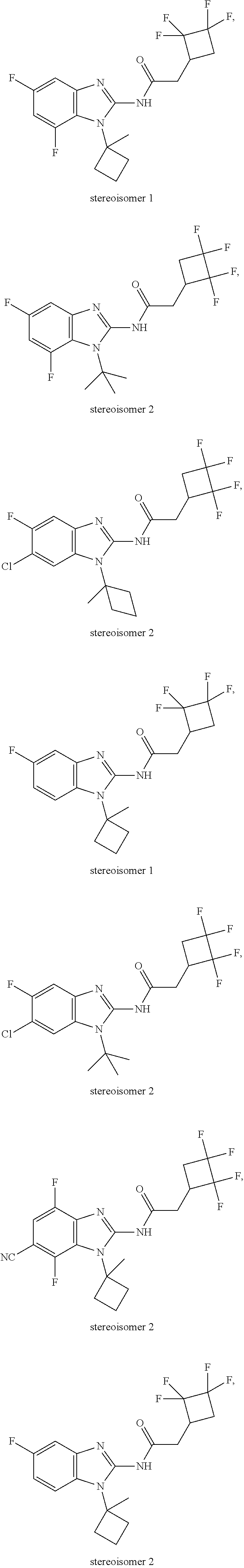

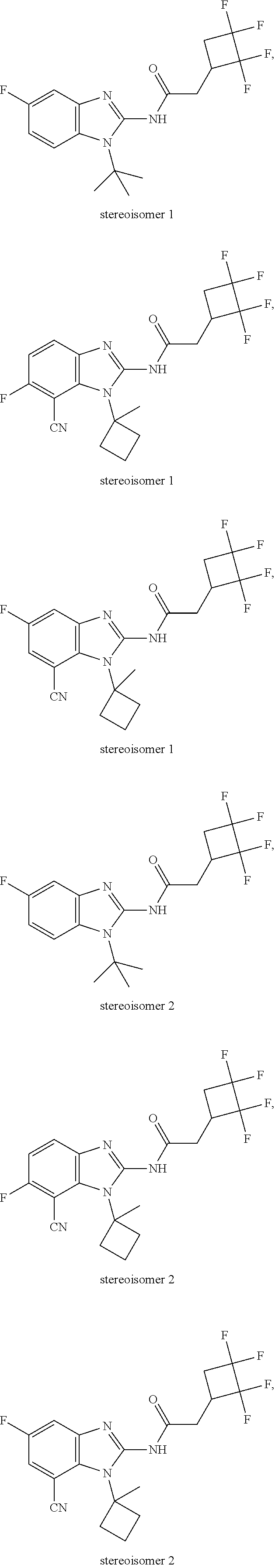 [SMILES: CC(C)(C)n1c(NC(=O)CC2CC(F)(F)C2(F)F)nc2cc(F)ccc21 ; CC1(n2c(NC(=O)CC3CC(F)(F)C3(F)F)nc3ccc(F)c(C#N)c32)CCC1 ; CC1(n2c(NC(=O)CC3CC(F)(F)C3(F)F)nc3cc(F)cc(C#N)c32)CCC1 ; CC(C)(C)n1c(NC(=O)CC2CC(F)(F)C2(F)F)nc2cc(F)ccc21 ; CC1(n2c(NC(=O)CC3CC(F)(F)C3(F)F)nc3ccc(F)c(C#N)c32)CCC1 ; CC1(n2c(NC(=O)CC3CC(F)(F)C3(F)F)nc3cc(F)cc(C#N)c32)CCC1]

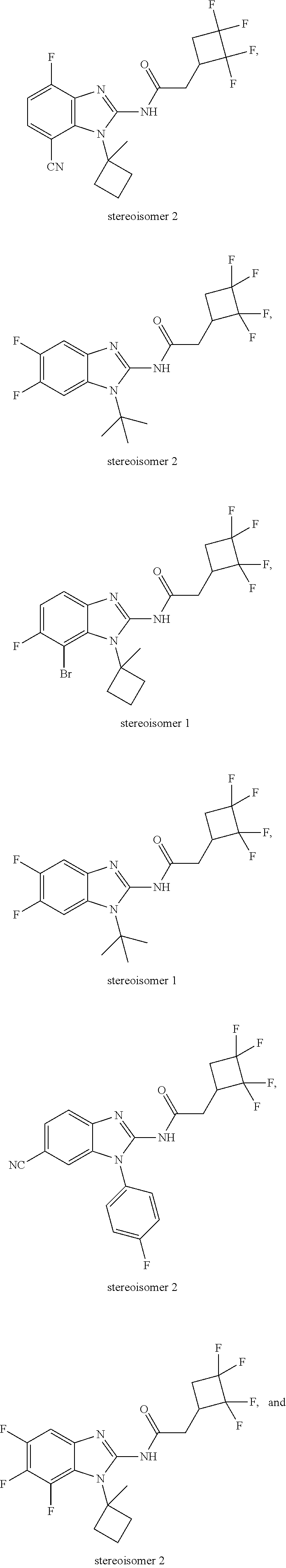

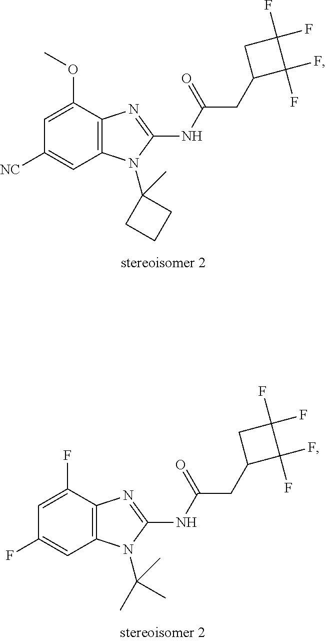 [SMILES: COc1cc(C#N)cc2c1nc(NC(=O)CC1CC(F)(F)C1(F)F)n2C1(C)CCC1 ; CC(C)(C)n1c(NC(=O)CC2CC(F)(F)C2(F)F)nc2c(F)cc(F)cc21]

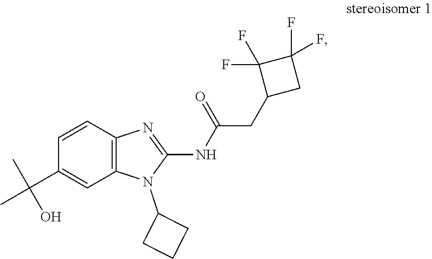 [SMILES: CC(C)(O)c1ccc2nc(NC(=O)CC3CC(F)(F)C3(F)F)n(C3CCC3)c2c1]

or a pharmaceutically acceptable salt thereof.

In further embodiments, the compound is:

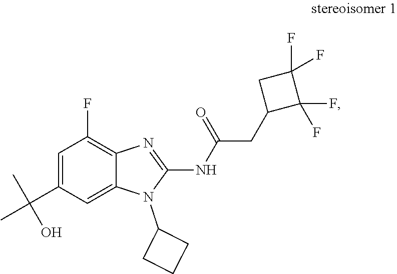 [SMILES: CC(C)(O)c1cc(F)c2nc(NC(=O)CC3CC(F)(F)C3(F)F)n(C3CCC3)c2c1]

or a pharmaceutically acceptable salt thereof.

In further embodiments, the compound is:

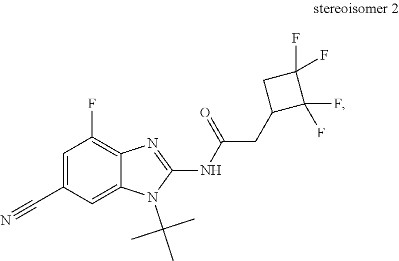 [SMILES: CC(C)(C)n1c(NC(=O)CC2CC(F)(F)C2(F)F)nc2c(F)cc(C#N)cc21]

or a pharmaceutically acceptable salt thereof.

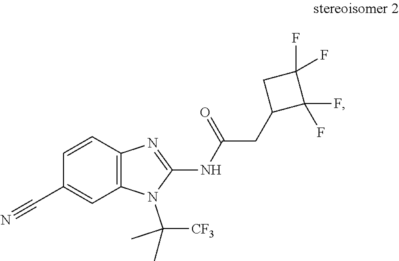 [SMILES: CC(C)(n1c(NC(=O)CC2CC(F)(F)C2(F)F)nc2ccc(C#N)cc21)C(F)(F)F]

or a pharmaceutically acceptable salt thereof.

In further embodiments, the compound is:

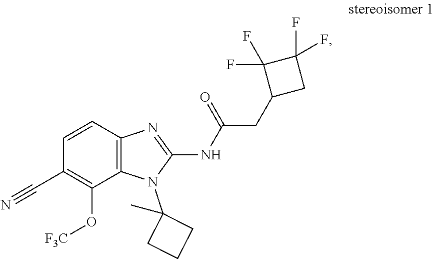 [SMILES: CC1(n2c(NC(=O)CC3CC(F)(F)C3(F)F)nc3ccc(C#N)c(OC(F)(F)F)c32)CCC1]

or a pharmaceutically acceptable salt thereof.

In further embodiments, the compound is:

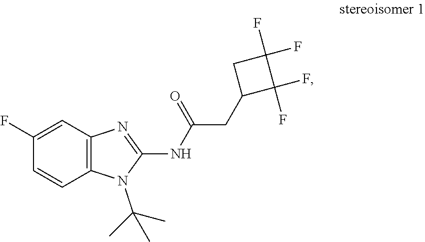 [SMILES: CC(C)(C)n1c(NC(=O)CC2CC(F)(F)C2(F)F)nc2cc(F)ccc21]

or a pharmaceutically acceptable salt thereof.

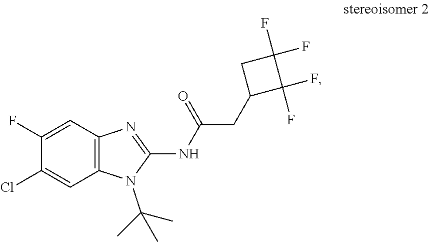 [SMILES: CC(C)(C)n1c(NC(=O)CC2CC(F)(F)C2(F)F)nc2cc(F)c(Cl)cc21]

or a pharmaceutically acceptable salt thereof.

In further embodiments, the compound is:

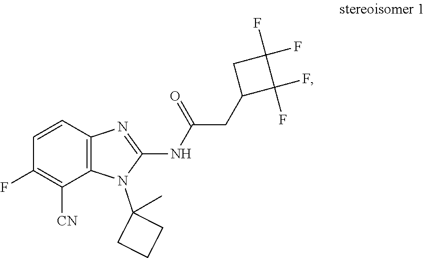 [SMILES: CC1(n2c(NC(=O)CC3CC(F)(F)C3(F)F)nc3ccc(F)c(C#N)c32)CCC1]

or a pharmaceutically acceptable salt thereof.

In further embodiments, the compound is:

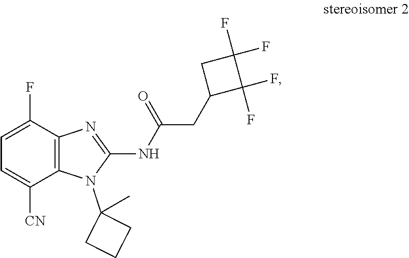 [SMILES: CC1(n2c(NC(=O)CC3CC(F)(F)C3(F)F)nc3c(F)ccc(C#N)c32)CCC1]

or a pharmaceutically acceptable salt thereof.

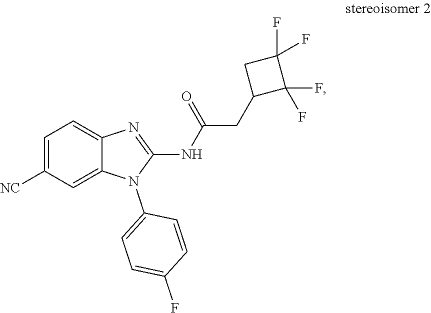 [SMILES: N#Cc1ccc2nc(NC(=O)CC3CC(F)(F)C3(F)F)n(-c3ccc(F)cc3)c2c1]

or a pharmaceutically acceptable salt thereof.

In further embodiments, the compound is:

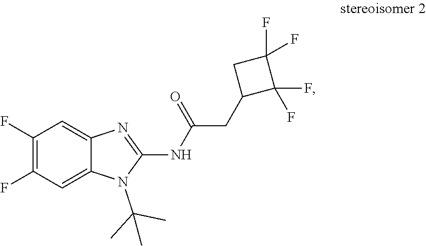 [SMILES: CC(C)(C)n1c(NC(=O)CC2CC(F)(F)C2(F)F)nc2cc(F)c(F)cc21]

or a pharmaceutically acceptable salt thereof.

In further embodiments, the compound is:

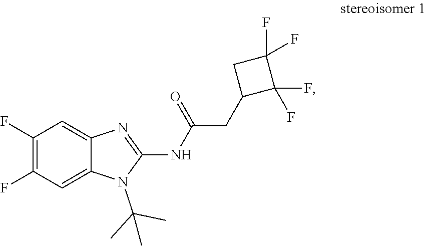 [SMILES: CC(C)(C)n1c(NC(=O)CC2CC(F)(F)C2(F)F)nc2cc(F)c(F)cc21]

or a pharmaceutically acceptable salt thereof.

In an embodiment of Formula 2, D is optionally substituted cyclobutyl, or t-butyl; A is C₂₋₈ alkyl, X is H, CF₃, or optionally substituted phenyl, Y is H or OH, R¹ is CN or C₁₋₄ hydroxyalkyl; and R², R³, and R⁴ are independently H, or F. In a further embodiment, R¹ is CN, —C(CH₃)₂OH, or —CH₂C(CH₃)₂OH. In a further embodiment, R² is F. In a further embodiment, R³ is H. In a further embodiment, R⁴ is H. In a further embodiment, R¹ is CN. In a further embodiment, R¹ is C₁₋₄ hydroxyalkyl. In a further embodiment, X is optionally substituted phenyl. In a further embodiment, X is CF₃. In a further embodiment, X is H. In a further embodiment, Y is H. In a further embodiment, Y is OH.

In an embodiment of Formula 2, D is cyclobutyl; A is C₁₋₈ alkyl, X is CF₃, Y is H, R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, CI or B; and R⁴ is H, —CH₃, or —CF₃.

In an embodiment of Formula 2, D is optionally substituted cyclobutyl, A is C₁₋₈ alkyl, X is CH₃, Y is H, R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃.

In an embodiment of Formula 2, D is t-butyl; A is C₁₋₈ alkyl; X is H; Y is H; R¹ is Cl, Br, CN, OCH₃, CF₃, —CO₂CH₂CH₃, C₁₋₄alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃.

In an embodiment of Formula 2, D is t-butyl; A is C₁₋₈ alkyl; X is CF₃; Y is H; R¹ is H, Cl, Br, CN, OCH₃, CF₃, —CO₂CH₂CH₃, C₁₋₄alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, CH₃, or CF₃.

In an embodiment of Formula 2, D is cyclobutyl; A is C₁₋₈ alkyl; X is H; Y is methyl (2,2,2-trifluoroethyl)amino; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —CH(CH₃)₂OH; R³ is H; and R⁴ is H, —CH₃, or —CF₃.

In an embodiment of Formula 2, D is optionally substituted cyclobutyl, optionally substituted phenyl, or optionally substituted C₂₋₅ alkyl, wherein the optional substituents are selected from —CH₃ and F; A is C1 alkyl, X is substituted cyclobutyl, wherein the substituent is F; Y is H; R¹ is selected from H, C3 hydroxyalkyl, CN, F, or Cl; R² is selected from H, CN, F, Br, or —OCF₃; R³ is selected from H, F, or —OCH₃; R⁴ is H or F; and wherein when X is substituted with 2 fluorine atoms, the fluorine atoms are not geminal; or a pharmaceutically acceptable salt thereof.

### Formula 3

In an embodiment, the Kv7 channel activator may be selected from one of the following compounds according to Formula 3. Such compounds are described in U.S. Pat. No. 8,293,911 issued on Oct. 23, 2012 and corresponding to U.S. application Ser. No. 11/894,877 filed Aug. 22, 2007; which is incorporated by reference in its entirety herein. In the case of any conflict of terminology in the context of Formula 3, this reference incorporated by reference herein controls.

In an embodiment, the Kv7 channel activator is a compound according to formula 3:

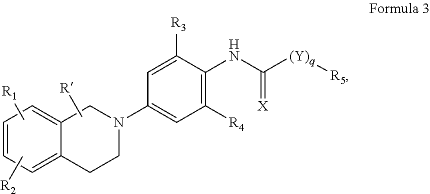

wherein, R₁ and R₂, vary independently, and are selected from the group consisting of H, CN, halogen, CH₂CN, OH, CH₂F, CHF₂, CF₃, CF₂CF₃, C₁-C₆ alkyl, C(═O)C₁-C₆ alkyl; NH—C₁-C₆ alkyl; N(C₁-C₆ alkyl)-C₁-C₆ alkyl, NHC(═O)C₁-C₆ alkyl, C(═O) N(CH₃)₂, C(═O)N(Et)₂, C(═O) NH₂, C(═O)NH—C₁-C₆alkyl, SO₂NH₂, NHSO₂—C₁-C₆ alkyl; C(═O)OC₁-C₆ alkyl, OC(═O)C₁-C₆ alkyl, OC₁-C₆ alkyl, SC₁-C₆ alkyl, C₃-C₆ cycloalkyl, (CH₂)ₘC₃-C₆ cycloalkyl, C₃-C₆ cycloalkenyl, (CH₂)ₘC₃-C₆ cycloalkenyl, C₂-C₆ alkenyl, C₂-C₆ alkynyl, Ar, (CH₂)ₘthienyl, (CH₂)ₘimidazolyl, (CH₂)ₘpyrazyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, and (CH₂)ₘpyrimidyl, Wherein m=zero, 1, or 2; Ar is a 5- to 10-member mono- or bicyclic aromatic group, optionally containing 1-4 ring heteroatoms selected independently from N, O, and S; or R₁ and R₂, together with the ring carbon atoms to which they are attached, form a 5- or 6-member fused ring; wherein said fused ring may be saturated, unsaturated, or aromatic, and said fused ring optionally contains one or two heteroatoms selected independently from the group consisting of O, N, and S; R′ is selected from the group consisting of H, halogen, phenyl, 2-(N, N-dimethylamino)ethyl, CF₃, OC₁-C₃ alkyl and C₁-C₃ alkyl; R₃ and R₄ vary independently, and are selected from the group consisting of H, CN, halogen, CF₃, OCF₃, OC₁-C₃ alkyl, and C₁-C₃ alkyl; X is O or S; Y is O or S; q=1 or zero; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆—C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar, wherein w=zero, 1, 2, or 3; Ar is a 5- to 10-member mono- or bicyclic aromatic group, optionally containing 1-4 ring heteroatoms selected independently from the group consisting of N, O, and S; and Reis selected from the group consisting of H or C₁-C₃ alkyl; where all cycloalkyl and cycloalkenyl optionally contain one or two ring heteroatoms selected independently from N, O, and S; wherein all alkyl, cycloalkyl, alkenyl, cycloalkenyl, heterocycloalkyl, heterocycloalkenyl, alkynyl, aryl, and heteroaryl groups in R₁, R₂, R′, R₃, R₄, R₅, R₆, and Ar are optionally substituted with one or two substituents selected independently from the group consisting of C₁-C₃ alkyl, halogen, OH, OEt, OMe, CN, CH₂F, OCF₃, and CF₃; and wherein, additionally, all cycloalkyl and heterocycloalkyl groups are optionally substituted with a carbonyl group.

In further embodiments, R₁ and R₂, vary independently, and are selected from the group consisting of H, halogen, CF₃, C₁-C₆ alkyl, C(═O)C₁-C₆ alkyl, C(═O)OC₁-C₆ alkyl, OC(═O)C₁-C₆ alkyl, OC₁-C₆ alkyl, SCH₃, C₃-C₆ cycloalkyl, (CH₂)ₘC₃-C₆ cycloalkyl, phenyl, pyridyl, pyrrolyl, thienyl, (CH₂)ₘphenyl, (CH₂)ₘpyrrolyl, and (CH₂)ₘpyridyl; wherein said cycloalkyl groups optionally contain one or two heteroatoms selected independently from the group consisting of O, N, and S; wherein said alkyl, cycloalkyl, phenyl, pyrrolyl, and pyridyl groups are optionally substituted with one or two groups selected, independently, from halogen, methyl, ethyl, or trifluoromethyl; and wherein m is zero, 1, or 2; R′ is selected from the group consisting of H, halogen, phenyl, 2-(N,N-dimethylamino)ethyl, CF₃, OC₁-C₃ alkyl and C₁-C₃ alkyl; R₃ and R₄ vary independently, and are selected from the group consisting of H, halogen, CF₃, OCF₃, OC₁-C₃ alkyl, and C₁-C₃ alkyl; X is O or S; Y is O or S; q=1 or 0; R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆—C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₇)_{w}CH₂Ar; wherein w=0-3; Ar is selected from the group consisting of phenyl, pyrimidyl, or pyridyl, and a 5-member heteroaromatic ring; wherein said heteroaromatic ring contains 1 or 2 ring heteroatoms selected independently from the group consisting of N, O, and S; and R₆ is selected from the group consisting of H and methyl; wherein all cycloalkyl and cycloalkenyl groups in R₅ optionally contain one or two ring heteroatoms selected independently from the group consisting of N, O, and S; and wherein all alkyl, cycloalkyl, alkenyl, cycloalkenyl, heterocycloalkyl, heterocycloalkenyl, alkynyl, aryl, and heteroaryl groups in R₁, R₂, R₃, R₄, R₅, R₆, and Ar are optionally substituted with one or two substituents selected independently from the group consisting of C₁-C₃ alkyl, halogen, OEt, OMe, and trifluoromethyl.

In further embodiments, R₁ and R₂, vary independently, and are selected from the group consisting of H, halogen, CF₃, C₁-C₆ alkyl, C(═O)C₁-C₆ alkyl, C(═O)OC₁-C₆ alkyl, OC(═O)C₁-C₆ alkyl, OC₁-C₆ alkyl, SCH₃, (CH₂)ₘcyclopropyl, (CH₂)ₘcyclobutyl, (CH₂)ₘcyclopentyl, (CH₂)ₘcyclohexyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, and (CH₂)ₘpyrimidyl; wherein said cyclopentyl and said cyclohexyl groups optionally contain one or two ring heteroatoms selected independently from the group consisting of O, N, and S; wherein said alkyl, cycloalkyl, phenyl, pyrrolyl, and pyridyl groups are optionally substituted with one or two groups selected, independently, from the group consisting of halogen, CH₃, ethyl, and CF₃; and m is zero, 1, or 2; R′ is selected from the group consisting of H, halogen, CF₃, and C₁-C₃ alkyl; R₃ and R₄ vary independently, and are selected from the group consisting of H, halogen, CF₃, OCF₃, OC₁-C₃ alkyl, and C₁-C₃ alkyl; X is O or S; Y is O; q=1 or 0; R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar; wherein w=0-3, Ar is selected from the group consisting of phenyl, pyridyl, and a 5-member heteroaromatic ring, wherein said heteroaromatic ring contains 1 or 2 ring heteroatoms selected independently from the group consisting of N, O, and S; R₆ is H or methyl; wherein all cycloalkyl and cycloalkenyl groups optionally contain one or two ring heteroatoms selected independently from the group consisting of N, O, and S; and wherein all alkyl, cycloalkyl, alkenyl, cycloalkenyl, heterocycloalkyl, heterocycloalkenyl, alkynyl, aryl, and heteroaryl groups in R₁, R₂, R₃, R₄, R₅, R₆, and Ar are optionally substituted with one or two substituents selected independently from the group consisting of C₁-C₃ alkyl, halogen, OMe, OEt, and CF₃.

In further embodiments, R₁ and R₂, vary independently, and are selected from the group consisting of H, halogen, CF₃, OC₁-C₃ alkyl, C₁-C₆ alkyl, C(═O)OC₁-C₃ alkyl, OC(═O)C₁-C₃ alkyl, and C(═O)C₁-C₃ alkyl; R′ is selected from the group consisting of H, F, CH₃, and ethyl; R₃ and R₄ vary independently, and are selected from the group consisting of H, F, Cl, CF₃, OCF₃, OC₁-C₃ alkyl, and C₁-C₃ alkyl; and R₅ is C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, or (CHR₆)_{w}Ar, CH₂(CHR₆)_{w}Ar, or (CHR₆)_{w}CH₂Ar.

In further embodiments, R₂ is H or F; R′ is H; R₃ is selected from the group consisting of H, CH₃, OCH₃, CF₃, OCF₃, and Cl; R₄ is selected from the group consisting of CH₃, OCH₃, CF₃, OCF₃, and Cl; and R₅ is C₃-C₆ alkyl or (CH₂)_{w}C₃-C₆ cycloalkyl.

In further embodiments, R₁ is halogen or CF₃; R₂ is H or F; R′ is H; R₃ and R₄ vary independently, and are selected from the group consisting of H, CH₃, OCH₃, CF₃, OCF₃, or Cl; and R₅ is selected from the group consisting of C₁-C₆ alkyl, (CHR₆)_{w}C3-C6 cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar.

In further embodiments, R₁ is halogen or CF₃; R₂ is H or F; R′ is H; R₃ and R₄ vary independently, and are selected from the group consisting of H, CH₃, OCH₃, CF₃, OCF₃, or Cl; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆—C₃-C₆ cycloalkyl, (CHR₆)_{w}C5-C6 cycloalkenyl, CH₂(CHR₆)_{w}C5-C6 cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar.

### Formula 4

In an embodiment, the Kv7 channel activator may be selected from one of the following compounds according to Formula 4. Such compounds are described in U.S. Pat. No. 8,293,911 issued on Oct. 23, 2012 and corresponding to U.S. application Ser. No. 11/894,877; which is incorporated by reference in its entirety herein. In the case of any conflict of terminology in the context of Formula 4, this reference incorporated by reference herein controls.

In an embodiment, the Kv7 channel activator is a compound according to formula 4:

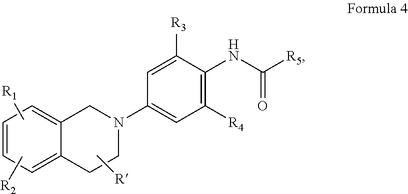

wherein, R₁ is selected from the group consisting of H, halogen, CN, CH₂CN, CF₃, C₁-C₆ alkyl, OCH₃, (C═O)OCH₃, O(C═O)CH₃, OCF₃, (CH₂)ₘC₃-C₆ cycloalkyl, phenyl, and pyridyl; R₂ is selected from the group consisting of H, F, OCH₃, CH₃, and CF₃; R₃ and R₄ vary independently, and are selected from the group consisting of H, F, Cl, CF₃, OCF₃, OC₁-C₃ alkyl, or C₁-C₃ alkyl; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar, wherein w=0-3; Ar is phenyl, furyl, pyrrolyl, oxazolyl, thiazolyl, thienyl, or pyridyl; and R₆ is C₁-C₃ alkyl; wherein all alkyl, cycloalkyl, aryl, and heteroaryl groups in R₁, R₂, R₃, R₄, R₅, R₆, and Ar are optionally substituted with one or two substituents selected independently from the group consisting of C₁-C₃ alkyl, halogen, OCH₃, OCH₂CH₃, CN, and CF₃.

In other embodiments, R₁ is selected from the group consisting of H, F, Cl, Br, CF₃, C₁-C₆ alkyl, OCH₃, CH₂OCH₃, CH₂CH₂OCH₃, CH₂OCH₂CH₃, and OCH₂CH₃; R′ is selected from the group consisting of H, CH₃, CH₂CH₃, or halogen; R₃ and R₄ vary independently, and are selected from the group consisting of H, F, Cl, CF₃, OCF₃, OCH₃, and CH₃; and R₅ is selected from the group consisting of C₁-C₆ alkyl, CH₂C₃-C₆ cycloalkyl, CH₂CH₂C₃-C₆ cycloalkyl, CH═CH—C₃-C₆ cycloalkyl, CH═CH—C₅-C₆ cycloalkenyl, CH₂C₅-C₆ cycloalkenyl, CH₂CH₂C₅-C₆ cycloalkenyl, C₂-C₆ alkenyl, and (CH₂)_{w}Ar; wherein w=1 or 2; Ar is selected from the group consisting of phenyl, oxazolyl, thiazolyl, isoxazolyl, isothiazolyl, furyl, thienyl, pyrrolyl, and pyridyl; wherein all alkyl, cycloalkyl, aryl, and heteroaryl groups in R₁, R₂, R₃, R₄, R₅, R₆, and Ar are optionally substituted with one or two substituents selected independently from the group consisting of CH₃, halogen, OCH₃, OCH₂CH₃, CN, and CF₃.

In other embodiments, R₁ is selected from the group consisting of H, F, Cl, Br, CF₃, C₁-C₆ alkyl, OCH₃, CH₂OCH₃, CH₂CH₂OCH₃, CH₂OCH₂CH₃, and OCH₂CH₃; R′ is selected from the group consisting of H, CH₃, CH₂CH₃, or halogen; R₃ and R₄ vary independently, and are selected from the group consisting of H, F, Cl, CF₃, OCF₃, OCH₃, and CH₃; and R₅ is selected from the group consisting of C₁-C₆ alkyl, CH₂C3-C6 cycloalkyl, CH₂CH₂C₃-C₆ cycloalkyl, CH═CH—C₃-C₆ cycloalkyl, CH═CH—C₅-C₆ cycloalkenyl, CH₂C₅-C₆ cycloalkenyl, CH₂CH₂C₅-C₆ cycloalkenyl, C₂-C₆ alkenyl, and (CH₂)_{w}Ar; wherein w=1 or 2; Ar is selected from the group consisting of phenyl, oxazolyl, thiazolyl, isoxazolyl, isothiazolyl, furyl, thienyl, pyrrolyl, and pyridyl; wherein all alkyl, cycloalkyl, aryl, and heteroaryl groups in R₁, R₂, R₃, R₄, R₅, R₆, and Ar are optionally substituted with one or two substituents selected independently from the group consisting of CH₃, halogen, OCH₃, OCH₂CH₃, CN, and CF₃. Wherein, R₁ is selected from the group consisting of F, CF₃, Cl, CH₃, CH₂CH₃, SCH₃, OCH₃, CH₂OCH₃, CH₂OCH₂CH₃, OCF₃, phenyl, thienyl, and H; R₂ is selected from the group consisting of H, F, Cl, and OCH₃; R′ is selected from the group consisting of H, F, CH₂CH₃, and CH₃; R₃ and R₄ vary independently, and are selected from the group consisting of H, Cl, CH₃, CF₃, OCH₃, and OCF₃; and R₅ is selected from the group consisting of C4-C6 alkyl, (CH₂)_{w}Ar, and (CH₂)_{w}C₅-C₆ cycloalkyl; wherein w is 1, 2, or 3.

In other embodiments, R₁ is selected from the group consisting of F, CF₃, Cl, CH₃, OCH₃, CH₂OCH₃, and H; R₂ is selected from the group consisting of H, F, CH₃, and Cl; R′ is H; R₅ is selected from the group consisting of H, Cl, CH₃, CF₃, OCH₃, and OCF₃; R₄ is selected from the group consisting of Cl, OCH₃, and CH₃; and R₅ is C₄-C₆ alkyl or 2-cyclopentyl ethyl.

In other embodiments, R₃ and R₄ are both CH₃ or both OCH₃; and R₅ is C₅-C₆ alkyl.

In other embodiments, R′ and R₂ are H; R₃ and R₄ are both methyl; and R₅ is C5-C6 alkyl or (CH₂)_{w}C5-C6 cycloalkyl; wherein w is 1, 2, or 3.

In other embodiments, the compound is selected from the group consisting of: N-(2-chloro-4-(3,4-dihydroisoquinolin-2 (1H)-yl)-6-(trifluoromethyl)phenyl)-3,3-dimethylbutanamide; N-(4-(3,4-dihydroisoquinolin-2 (1H)-yl)-2,6-dimethylphenyl)-3,3-dimethylbutanamide; N-(2-chloro-4-(3,4-dihydroisoquinolin-2 (1H)-yl)-6-(trifluoromethyl)phenyl)-3-cyclopentylpropanamide; N-(2-chloro-4-(6-fluoro-3,4-dihydroisoquinolin-2 (1H)-yl)-6-(trifluoromethylphenyl)-3,3-dimethylbutanamide; N-[2-chloro-4-(3,4-dihydro-1H-isoquinolin-2-yl)-6-methyl phenyl]-3,3-dimethylbutanamide; N-[2-chloro-4-(6-fluoro-3,4-dihydro-1H-isoquinolin-2-yl)-6-trifluoromethyl phenyl]-3-cyclopentylpropionamide; N-[2,6-dimethyl-4-(6-trifluoromethyl-3,4-dihydro-1H-isoquinolin-2-yl)-phenyl]-3,3-dimethylbutanamide; N-[2-chloro-6-trifluoromethyl-4-(6-trifluoromethyl-3,4-dihydro-1H-isoquinolin-2-yl)-phenyl]-3,3-dimethylbutanamide; N-[2-chloro-4-(6-chloro-3,4-dihydro-1H-isoquinolin-2-yl)-6-trifluoromethyl phenyl]-3,3-dimethylbutanamide; N-[4-(6-chloro-3,4-dihydro-1H-isoquinolin-2-yl)-2,6-dimethyl-phenyl]-3,3-dimethylbutanamide; N-[4-(6-fluoro-3,4-dihydro-1H-isoquinolin-2-yl)-2,6-dimethyl phenyl]-3,3-dimethylbutanamide; N-[2-chloro-4-(7-fluoro-3,4-dihydro-1H-isoquinolin-2-yl)-6-trifluoromethyl-phenyl]-3,3-dimethylbutanamide; N-[4-(7-fluoro-3,4-dihydro-1H-isoquinolin-2-yl)-2,6-dimethyl-phenyl]-3,3-dimethylbutanamide; N-[2-chloro-4-(6-fluoro-3,4-dihydro-1H-isoquinolin-2-yl)-6-methylphenyl]-3,3-dimethylbutanamide; N-[2-chloro-4-(7-fluoro-3,4-dihydro-1H-isoquinolin-2-yl)-6-methylphenyl]-3,3-dimethylbutanamide; N-[2-chloro-6-methyl-4-(6-trifluoromethyl-3,4-dihydro-1H-isoquinolin-2-yl)-phenyl]-3,3-dimethylbutanamide; N-[2-chloro-4-(6-chloro-3,4-dihydro-1H-isoquinolin-2-yl)-6-methyl-phenyl]-3,3-dimethylbutanamide; N-[2-chloro-4-(6-fluoro-3,4-dihydro-1H-isoquinolin-2-yl)-phenyl]-3,3-dimethylbutanamide; N-[4-(6-fluoro-3,4-dihydro-1H-isoquinolin-2-yl)-2-methyl-phenyl]-3,3-dimethylbutanamide; N-[4-(6-fluoro-3,4-dihydro-1H-isoquinolin-2-yl)-2-trifluoromethylphenyl]-3,3-dimethylbutanamide; N-[2-chloro-4-(6-trifluoromethyl-3,4-dihydro-1H-isoquinolin-2-yl)-phenyl]-3,3-dimethylbutanamide; N-[4-(7-fluoro-3,4-dihydro-1H-isoquinolin-2-yl)-2-trifluoromethyl-phenyl]-3,3-dimethylbutanamide; 3,3-dimethyl-N-[2-trifluoromethyl-4-(7-trifluoromethyl-3,4-dihydro-1H-isoquinolin-2-yl)-phenyl]butanamide; N-[4-(6-methoxy-3,4-dihydro-1H-isoquinolin-2-yl)-2,6-dimethyl-phenyl]-3,3-dimethylbutanamide; N-[4-(3,4-dihydro-1H-isoquinolin-2-yl)-2-methoxy-6-methyl-phenyl]-3,3-dimethylbutanamide; N-[2-chloro-4-(3,4-dihydro-1H-isoquinolin-2-yl)-6-trifluoromethoxy-phenyl]-3,3-dimethylbutanamide; N-[4-(3,4-dihydro-M-isoquinolin-2-yl)-2,6-dimethoxy-phenyl]-3,3-dimethylbutanamide; N-[2,6-dimethyl-4-(7-trifluoromethyl-3,4-dihydro-1H-isoquinolin-2-yl)-phenyl]-3,3-dimethyl butanamide; N-[2,6-Dimethyl-4-(6-trifluoromethyl-3,4-dihydro-1H-isoquinolin-2-yl)-phenyl]-3,3-dimethyl-thiobutanamide; [2,6-Dimethyl-4-(6-trifluoromethyl-3,4-dihydro-1H-isoquinolin-2-yl)-phenyl]-carbamic acid ethyl ester; and N-[2,6-Dimethyl-4-(7-trifluoromethyl-3,4-dihydro-1H-isoquinolin-2-yl)-phenyl]-3,3-dimethyl butanamide.

### Formula 5

In another embodiment, the Kv7 channel activator may be selected from one of the following compounds. Such compounds are described in U.S. Pat. No. 8,993,593 issued on Mar. 31, 2015 and corresponding to U.S. application Ser. No. 12/698,070 filed Feb. 1, 2010; US Publication No. US20220265634A1, published Aug. 25, 2022 and corresponding to U.S. application Ser. No. 17/668,340 filed Feb. 9, 2022; which are incorporated by reference in their entirety herein. In the case of any conflict of terminology in the context of Formula 5, these references incorporated by reference herein control.

In an embodiment, the Kv7 channel activator is a compound according to formula 5:

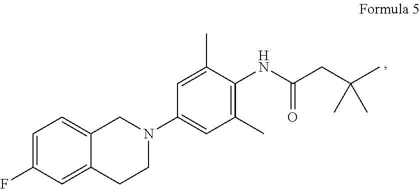 [SMILES: Cc1cc(N2CCc3cc(F)ccc3C2)cc(C)c1NC(=O)CC(C)(C)C]

optionally wherein the compound is substituted at any position.

### Formula 6

In another embodiment, the Kv7 channel activator may be the following compound (ezogabine, also known as retigabine) or a pharmaceutically acceptable salt thereof. Ezogabine is a compound according to formula 6: In the case of any conflict of terminology in the context of Formula 6, these references incorporated by reference herein control.

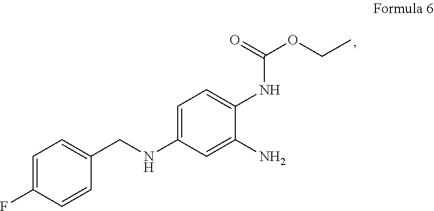 [SMILES: CCOC(=O)Nc1ccc(NCc2ccc(F)cc2)cc1N]

or, optionally, wherein the compound is substituted at any position.

### Formula 7

In an embodiment, the Kv7 channel activator may be selected from one of the following compounds according to Formula 7. Such compounds are described in U.S. Pat. No. 10,526,328 issued on Jan. 7, 2020 and corresponding to U.S. application Ser. No. 16/124,853 filed Sep. 7, 2018; U.S. Pat. No. 10,106,536 issued on Oct. 23, 2018 and corresponding to U.S. application Ser. No. 15/591,844 filed on May 10, 2017; US U.S. Pat. No. 9,650,376 issued on May 16, 2017, and corresponding to U.S. application Ser. No. 14/776,271 filed Mar. 17, 2014; US Publication No. US20220060208A1, published Feb. 24, 2022 and corresponding to U.S. application Ser. No. 17/277,145 filed Jan. 14, 2019; which are incorporated by reference in their entirety herein. In the case of any conflict of terminology in the context of Formula 7, these references incorporated by reference herein control.

In an embodiment, the Kv7 channel activator is a compound according to formula 7:

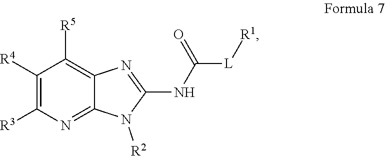

wherein, L is CH₂; R¹ is optionally substituted cyclic C₃H₅, wherein the optional substituent of R¹ is CF₃; R² is optionally substituted cyclobutyl; R³ is optionally substituted C₃ alkyl, wherein the optional substituent of R³ is OH; R⁴ is H; and R⁵ is H;
or
wherein, L is CH₂; R¹ is optionally substituted C₂ alkyl, wherein the optional substituents of R¹ are independently CF₃ or CH₃; R² is optionally substituted cyclobutyl; R³ is optionally substituted C₃ alkyl, wherein the optional substituent of R³ is OH; R⁴ is H; and R⁵ is H;
or
wherein, wherein L is CH₂, CF₂, CHCH₃, CH₂CH₂, C₃H₆, CH₂O, C₂H₄O, or C₃H₆O with the O of CH₂O, C₂H₄O, or C₃H₆O bonded with R¹; wherein R¹ is optionally substituted C₁₋₂ alkyl, optionally substituted C₅₋₁₀ cycloalkyl, optionally substituted C₁₋₁₂—O-alkyl, optionally substituted C₆₋₁₀ aryl, optionally substituted C₆₋₁₀—O-aryl, or optionally substituted C₂₋₉ heterocyclyl, wherein the optional substituents of R¹ are independently R^{A}, F, Cl, CN, OR^{A}, CF₃, NR^{A}R^{B}, COR^{A}, CO₂R^{A}, OCOR^{A}, NR^{A}COR^{B}, CONR^{A}R^{B}, wherein R^{A} and R^{B} are independently H or C₁₋₁₂ alkyl; wherein R² is optionally substituted C₂₋₄ acyclic alkyl, optionally substituted cyclobutyl, optionally substituted C₆₋₁₀ aryl, or optionally substituted C₂₋₉ heterocyclyl, wherein the optional substituents of R² are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ alkylthio, or C₁₋₆ alkylsulfonyl; wherein R³, R⁴, and R⁵ are independently H, F, Cl, Br, I, CN, optionally substituted C₁₋₁₂ alkyl, optionally substituted C₁₋₁₂-O-alkyl, optionally substituted C₂₋₉ heterocyclyl, optionally substituted C₆₋₁₀ aryl, optionally substituted C₂₋₉-O-heterocyclyl, optionally substituted C₆₋₁₀ O-aryl, optionally substituted C₁₋₁₂ acylamino, optionally substituted C₁₋₁₂ aminoacyl, or optionally substituted C₁₋₁₂ aminoalkyl, wherein the optional substituents of R³, R⁴, or R⁵ are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ acylamino, C₁₋₆ alkylthio, or C₁₋₆ alkylsulfonyl.

In further embodiments, R² is optionally substituted C₄H₉, optionally substituted cyclobutyl, optionally substituted C6-10 aryl, or optionally substituted C₂₋₉ heterocyclyl.

In further embodiments, R² is optionally substituted C₄H₉, or optionally substituted cyclobutyl.

In further embodiments, R² is optionally substituted phenyl, or optionally substituted C₂₋₉ heterocyclyl.

In further embodiments, R¹ is optionally substituted phenyl.

In further embodiments, R¹ is optionally substituted phenyl.

In further embodiments, R¹ is optionally substituted cyclopentyl or optionally substituted cyclohexyl.

In further embodiments, R¹ is C₂₋₄-O-alkyl.

In further embodiments, R¹ is optionally substituted tetrahydrofuranyl or optionally substituted tetrahydro-2H-pyranyl.

In further embodiments, R¹ is selected from the group consisting of:

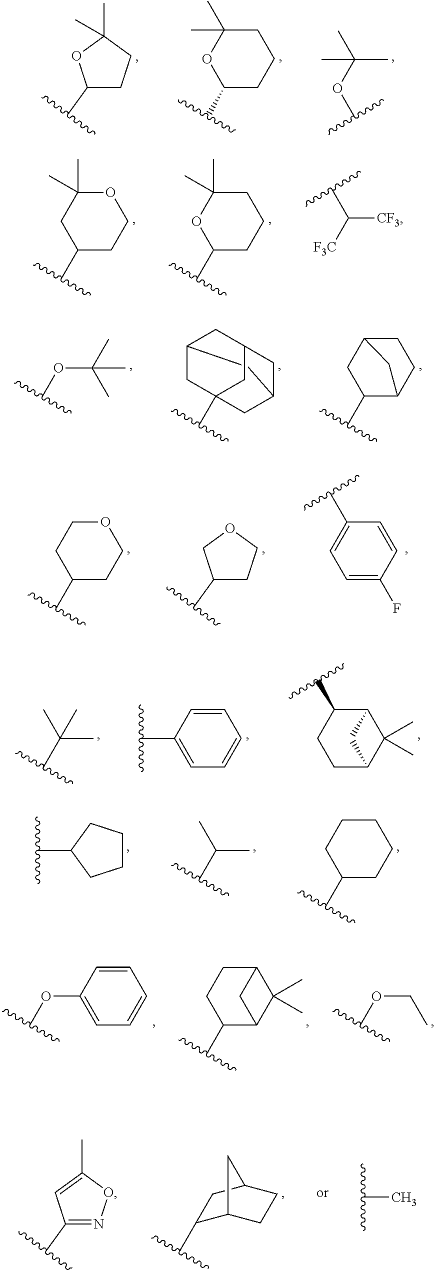

In further embodiments, R² is selected from the group consisting of:

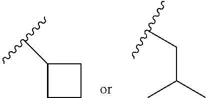

In further embodiments, R³ is CF₃, Cl, CN, OCH₃, or H.

In further embodiments, R¹ is selected from the group consisting of:

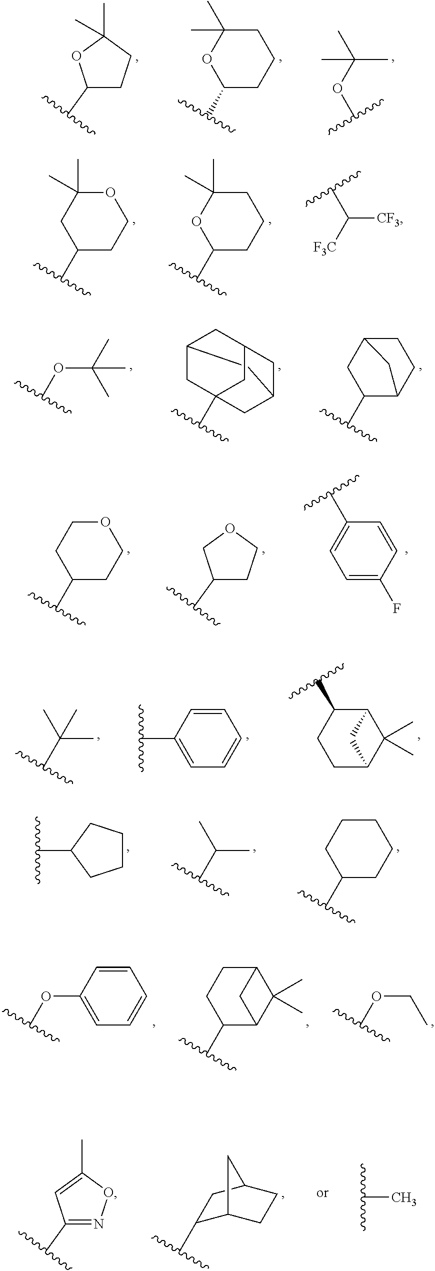

In further embodiments, R⁴ is CH₃, CF₃, Cl, or H.

In further embodiments, R² is selected from the group consisting of:

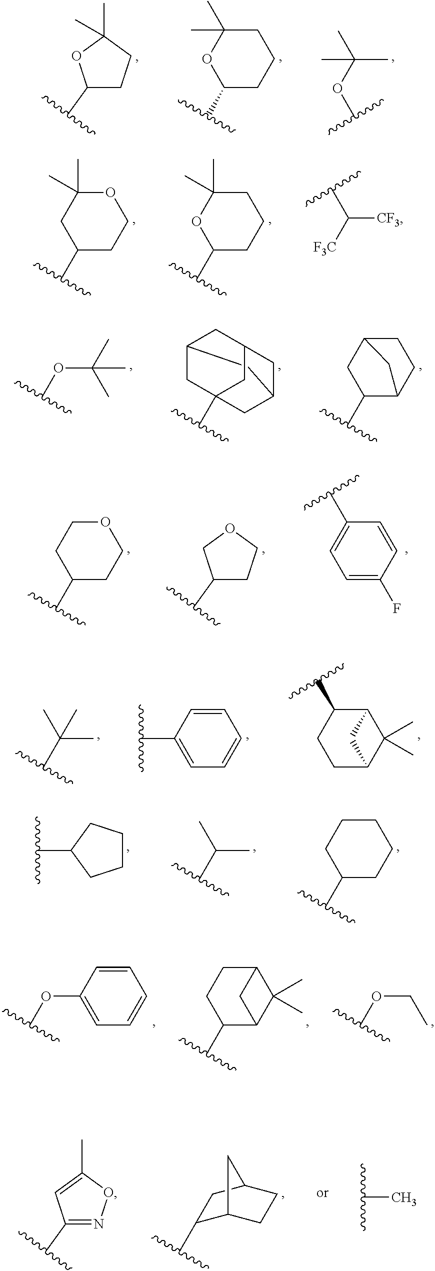

In further embodiments, the Kv7 channel activator is selected from the group consisting of:

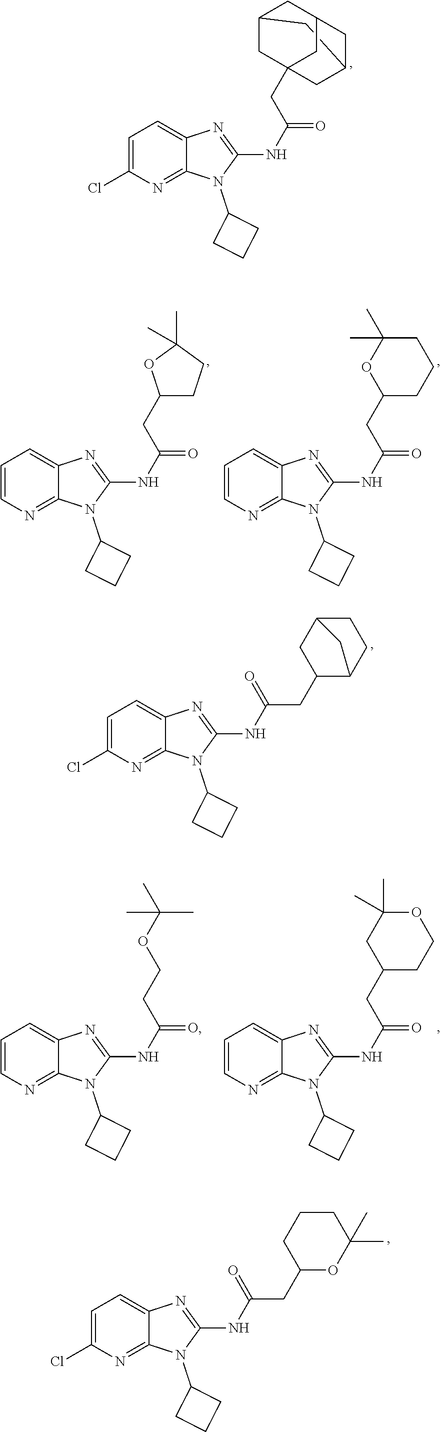

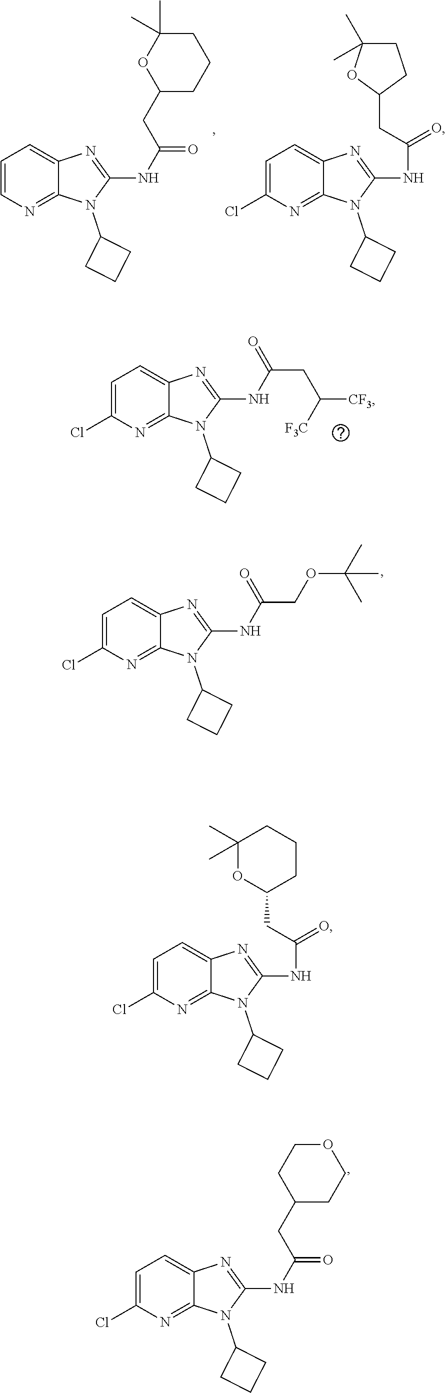 [SMILES: CC1(C)CCCC(CC(=O)Nc2nc3cccnc3n2C2CCC2)O1 ; CC1(C)CCC(CC(=O)Nc2nc3ccc(Cl)nc3n2C2CCC2)O1 ; O=C(CC(C(F)(F)F)C(F)(F)F)Nc1nc2ccc(Cl)nc2n1C1CCC1 ; CC(C)(C)OCC(=O)Nc1nc2ccc(Cl)nc2n1C1CCC1 ; CC1(C)CCC[C@H](CC(=O)Nc2nc3ccc(Cl)nc3n2C2CCC2)O1 ; O=C(CC1CCOCC1)Nc1nc2ccc(Cl)nc2n1C1CCC1]

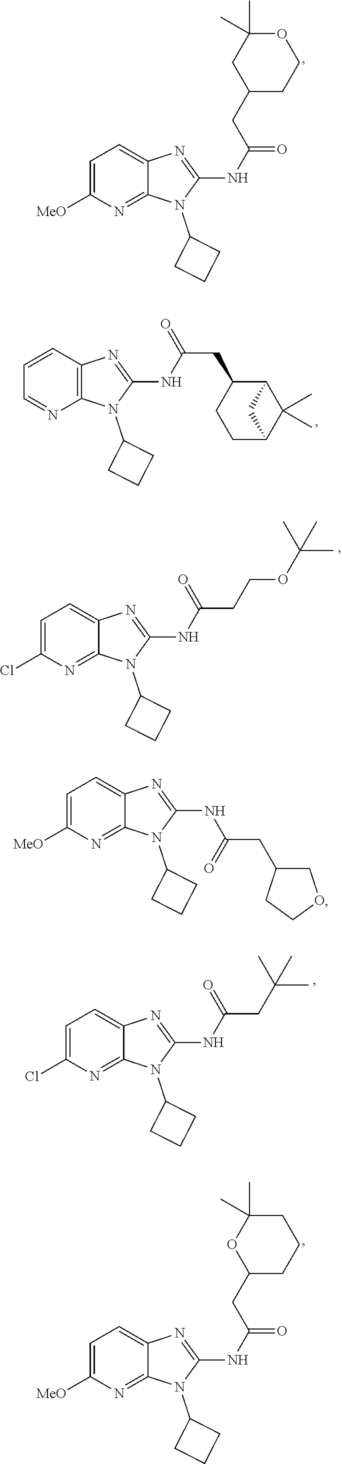

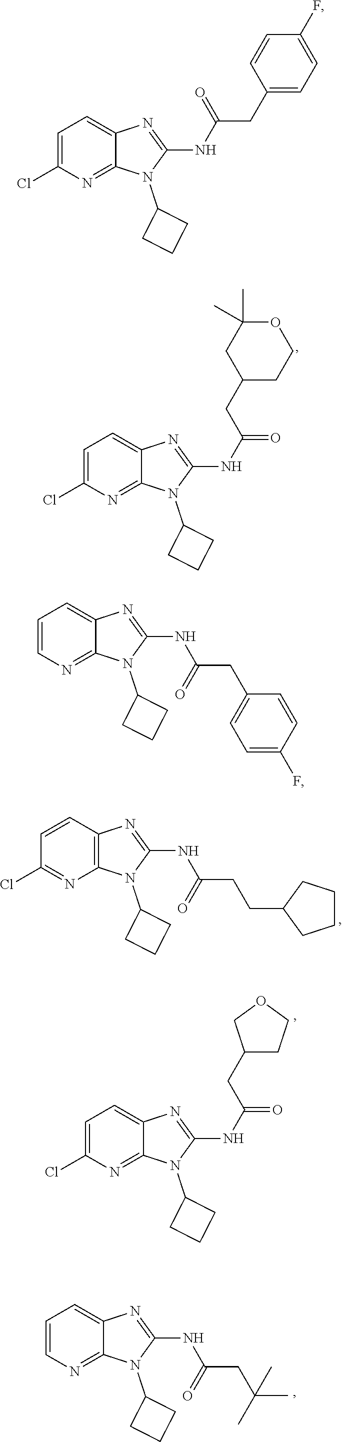 [SMILES: O=C(Cc1ccc(F)cc1)Nc1nc2ccc(Cl)nc2n1C1CCC1 ; CC1(C)CC(CC(=O)Nc2nc3ccc(Cl)nc3n2C2CCC2)CCO1 ; O=C(Cc1ccc(F)cc1)Nc1nc2cccnc2n1C1CCC1 ; O=C(CCC1CCCC1)Nc1nc2ccc(Cl)nc2n1C1CCC1 ; O=C(CC1CCOC1)Nc1nc2ccc(Cl)nc2n1C1CCC1 ; CC(C)(C)CC(=O)Nc1nc2cccnc2n1C1CCC1]

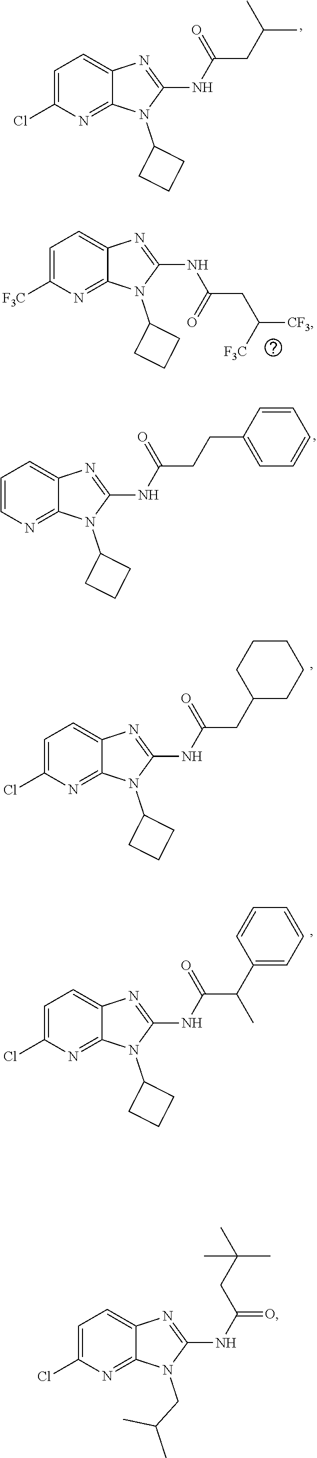 [SMILES: CC(C)CC(=O)Nc1nc2ccc(Cl)nc2n1C1CCC1 ; O=C(CC(C(F)(F)F)C(F)(F)F)Nc1nc2ccc(C(F)(F)F)nc2n1C1CCC1 ; O=C(CCc1ccccc1)Nc1nc2cccnc2n1C1CCC1 ; O=C(CC1CCCCC1)Nc1nc2ccc(Cl)nc2n1C1CCC1 ; CC(C(=O)Nc1nc2ccc(Cl)nc2n1C1CCC1)c1ccccc1 ; CC(C)Cn1c(NC(=O)CC(C)(C)C)nc2ccc(Cl)nc21]

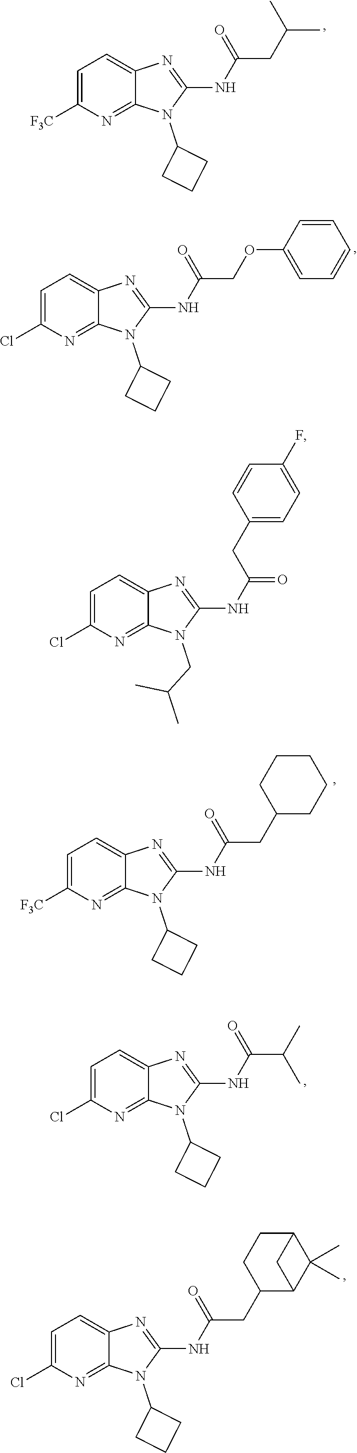 [SMILES: CC(C)CC(=O)Nc1nc2ccc(C(F)(F)F)nc2n1C1CCC1 ; O=C(COc1ccccc1)Nc1nc2ccc(Cl)nc2n1C1CCC1 ; CC(C)Cn1c(NC(=O)Cc2ccc(F)cc2)nc2ccc(Cl)nc21 ; O=C(CC1CCCCC1)Nc1nc2ccc(C(F)(F)F)nc2n1C1CCC1 ; CC(C)C(=O)Nc1nc2ccc(Cl)nc2n1C1CCC1 ; CC1(C)C2CCC(CC(=O)Nc3nc4ccc(Cl)nc4n3C3CCC3)C1C2]

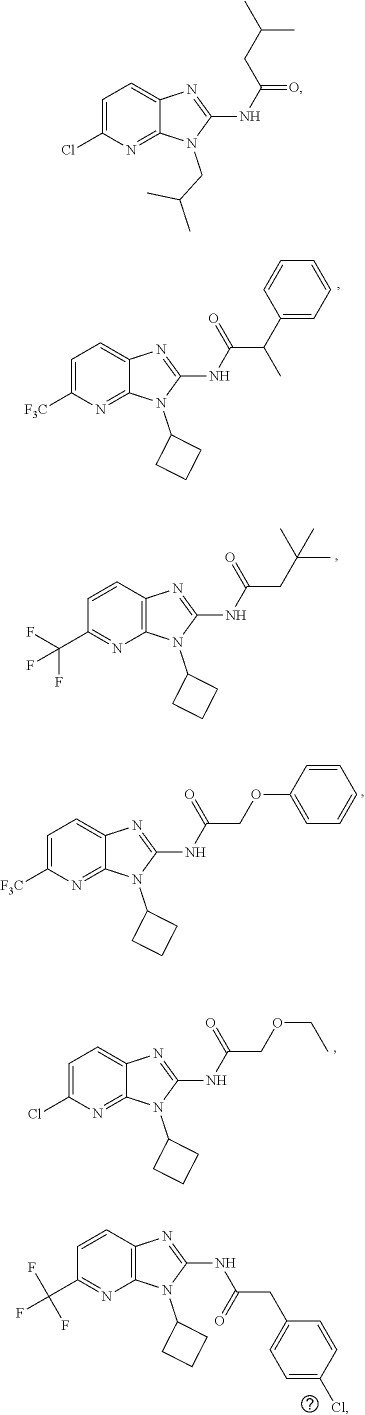 [SMILES: CC(C)CC(=O)Nc1nc2ccc(Cl)nc2n1CC(C)C ; CC(C(=O)Nc1nc2ccc(C(F)(F)F)nc2n1C1CCC1)c1ccccc1 ; CC(C)(C)CC(=O)Nc1nc2ccc(C(F)(F)F)nc2n1C1CCC1 ; O=C(COc1ccccc1)Nc1nc2ccc(C(F)(F)F)nc2n1C1CCC1 ; CCOCC(=O)Nc1nc2ccc(Cl)nc2n1C1CCC1 ; O=C(Cc1ccc(Cl)cc1)Nc1nc2ccc(C(F)(F)F)nc2n1C1CCC1]

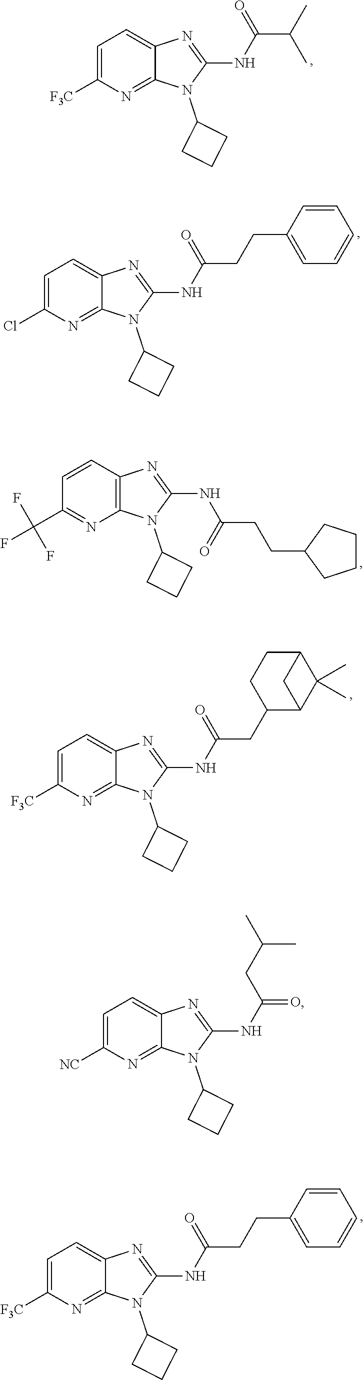 [SMILES: CC(C)C(=O)Nc1nc2ccc(C(F)(F)F)nc2n1C1CCC1 ; O=C(CCc1ccccc1)Nc1nc2ccc(Cl)nc2n1C1CCC1 ; O=C(CCC1CCCC1)Nc1nc2ccc(C(F)(F)F)nc2n1C1CCC1 ; CC1(C)C2CCC(CC(=O)Nc3nc4ccc(C(F)(F)F)nc4n3C3CCC3)C1C2 ; CC(C)CC(=O)Nc1nc2ccc(C#N)nc2n1C1CCC1 ; O=C(CCc1ccccc1)Nc1nc2ccc(C(F)(F)F)nc2n1C1CCC1]

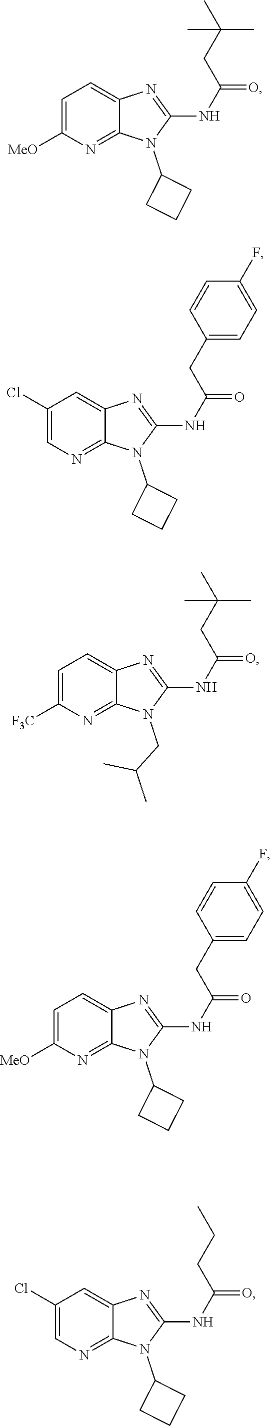 [SMILES: COc1ccc2nc(NC(=O)CC(C)(C)C)n(C3CCC3)c2n1 ; O=C(Cc1ccc(F)cc1)Nc1nc2cc(Cl)cnc2n1C1CCC1 ; CC(C)Cn1c(NC(=O)CC(C)(C)C)nc2ccc(C(F)(F)F)nc21 ; COc1ccc2nc(NC(=O)Cc3ccc(F)cc3)n(C3CCC3)c2n1 ; CCCC(=O)Nc1nc2cc(Cl)cnc2n1C1CCC1]

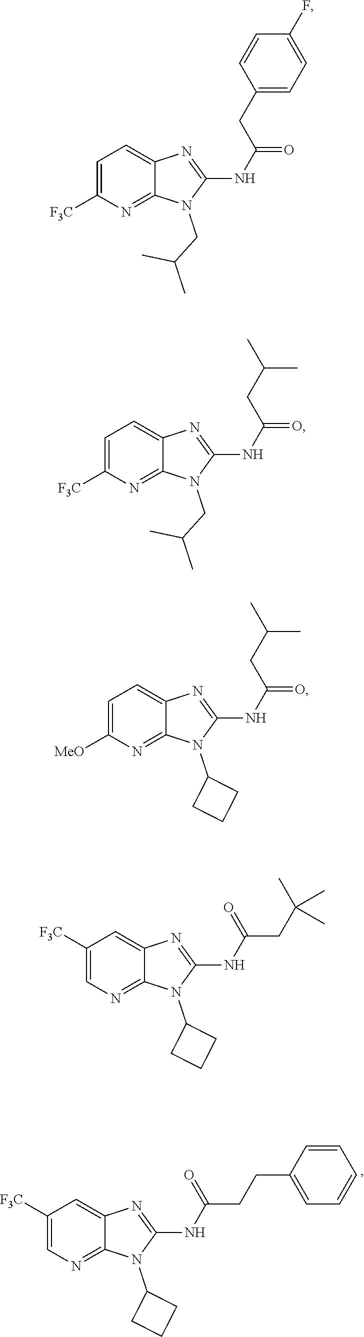 [SMILES: CC(C)Cn1c(NC(=O)Cc2ccc(F)cc2)nc2ccc(C(F)(F)F)nc21 ; CC(C)CC(=O)Nc1nc2ccc(C(F)(F)F)nc2n1CC(C)C ; COc1ccc2nc(NC(=O)CC(C)C)n(C3CCC3)c2n1 ; CC(C)(C)CC(=O)Nc1nc2cc(C(F)(F)F)cnc2n1C1CCC1 ; O=C(CCc1ccccc1)Nc1nc2cc(C(F)(F)F)cnc2n1C1CCC1]

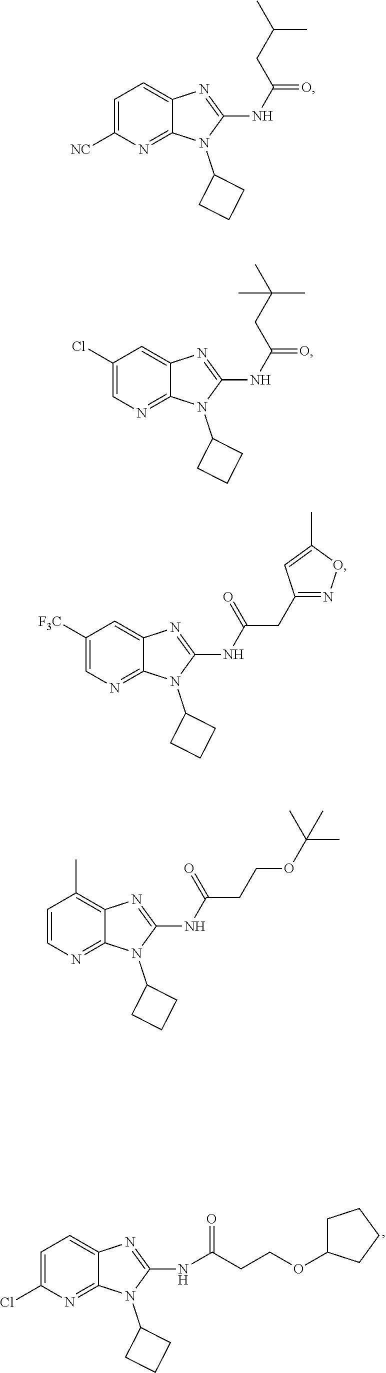 [SMILES: CC(C)CC(=O)Nc1nc2ccc(C#N)nc2n1C1CCC1 ; CC(C)(C)CC(=O)Nc1nc2cc(Cl)cnc2n1C1CCC1 ; Cc1cc(CC(=O)Nc2nc3cc(C(F)(F)F)cnc3n2C2CCC2)no1 ; Cc1ccnc2c1nc(NC(=O)CCOC(C)(C)C)n2C1CCC1 ; O=C(CCOC1CCCC1)Nc1nc2ccc(Cl)nc2n1C1CCC1]

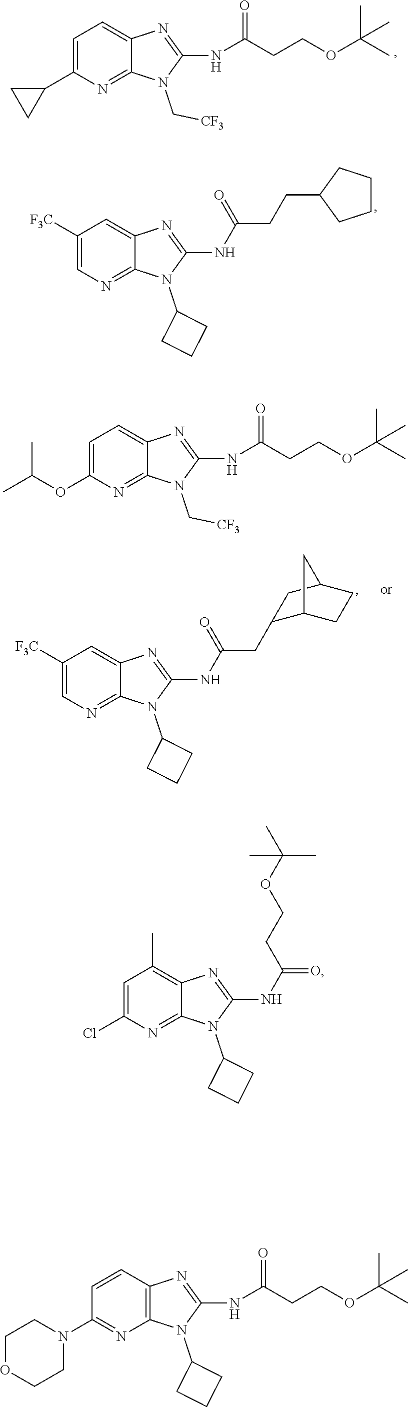

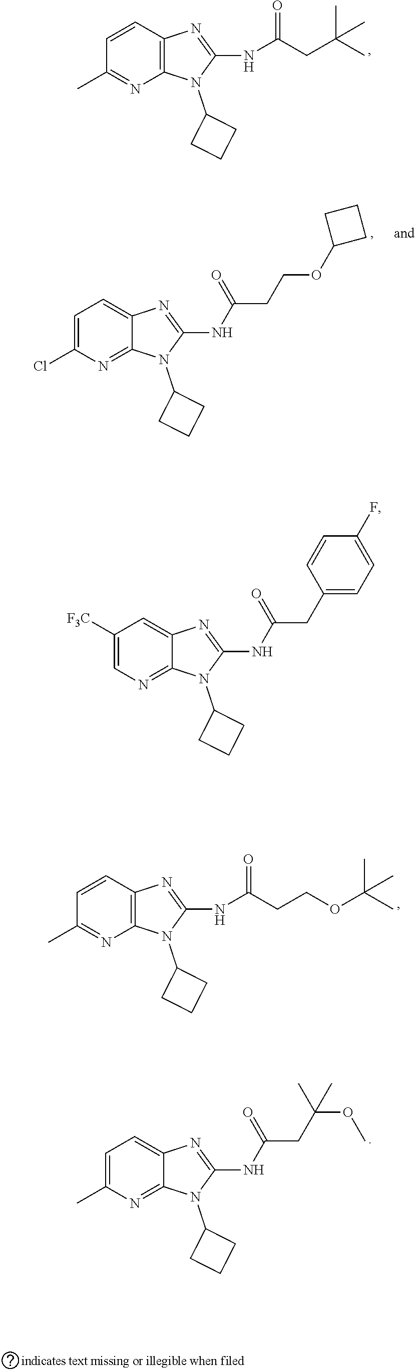

In further embodiments, the Kv7 channel activator is selected from the group consisting of:

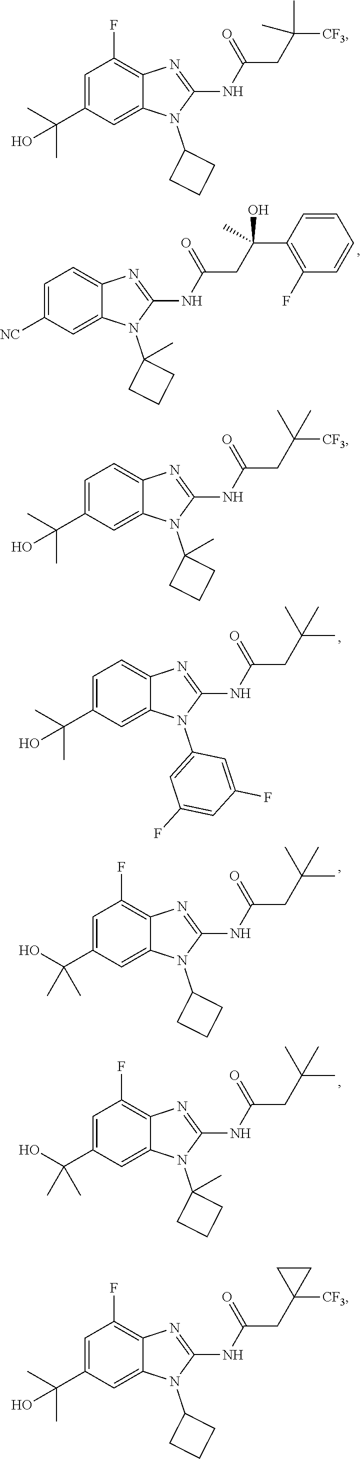

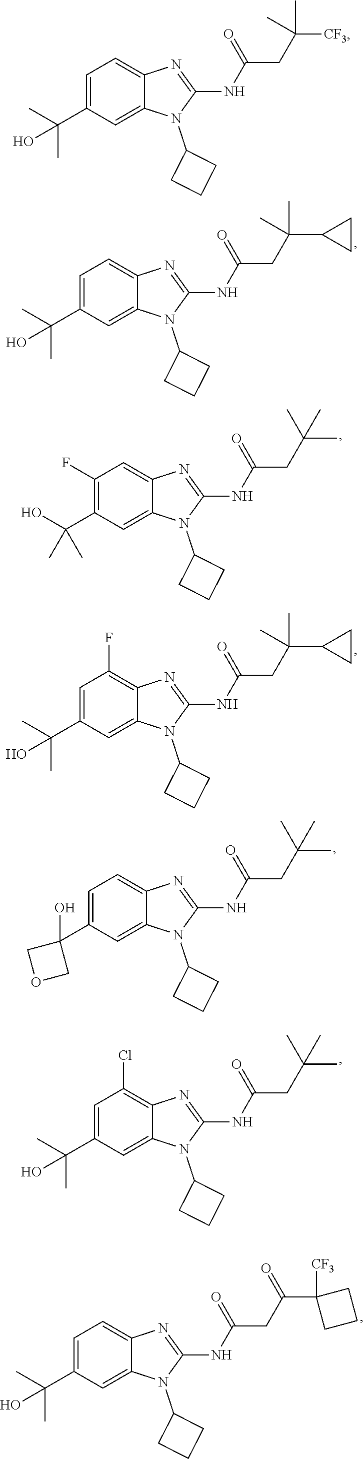

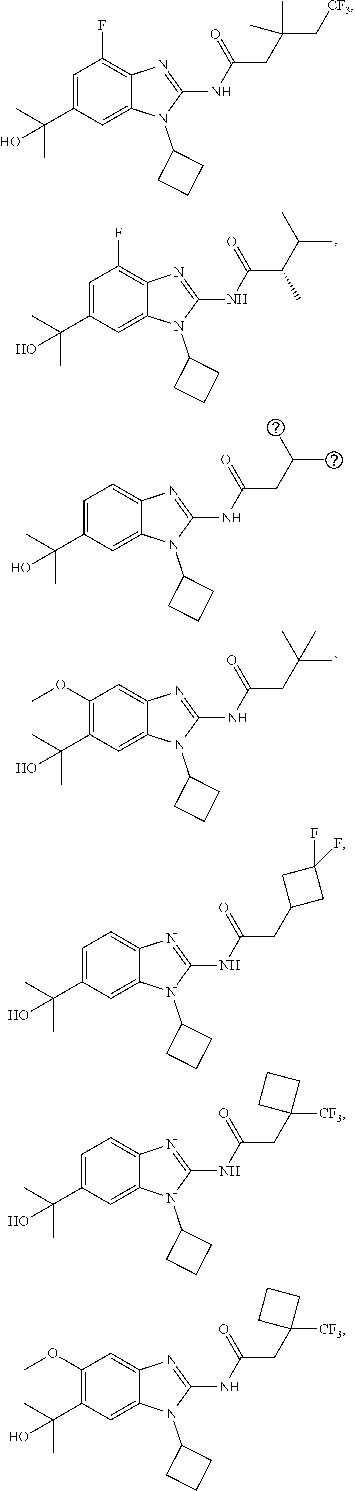

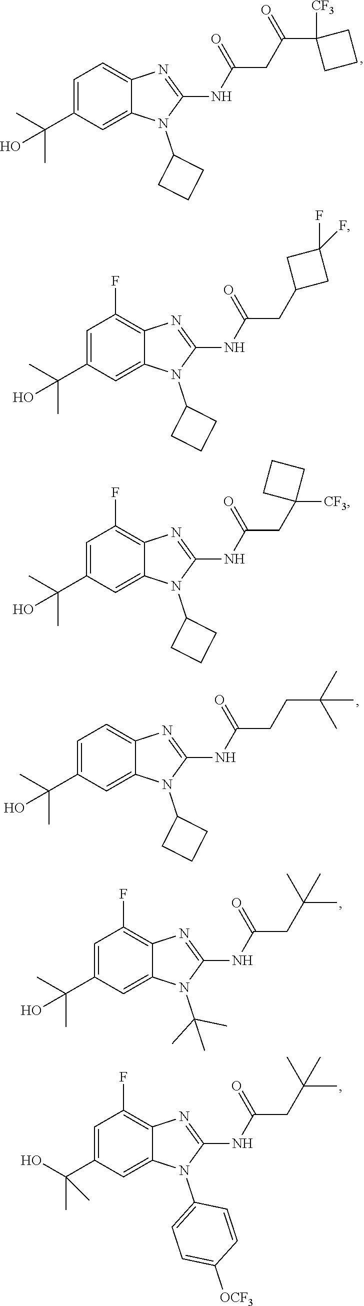

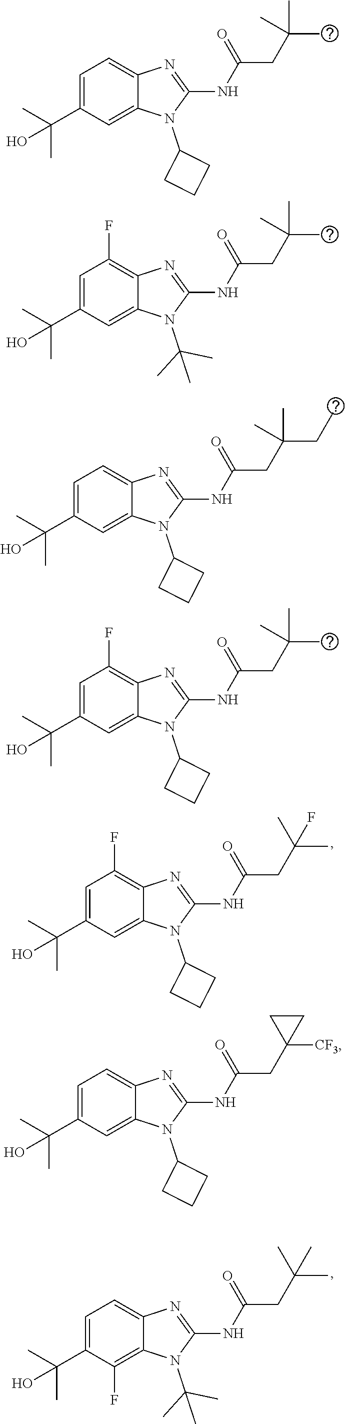

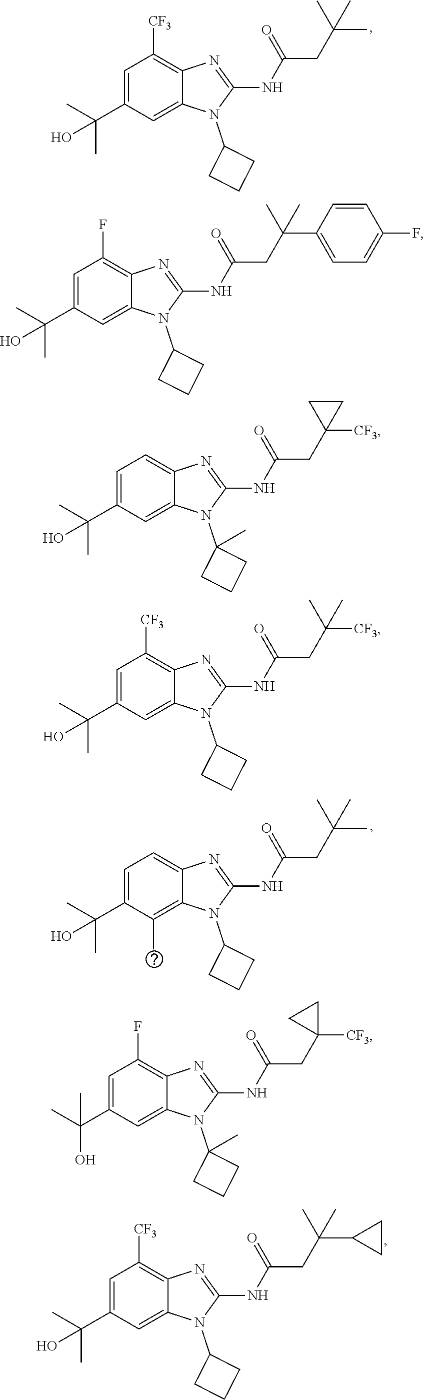

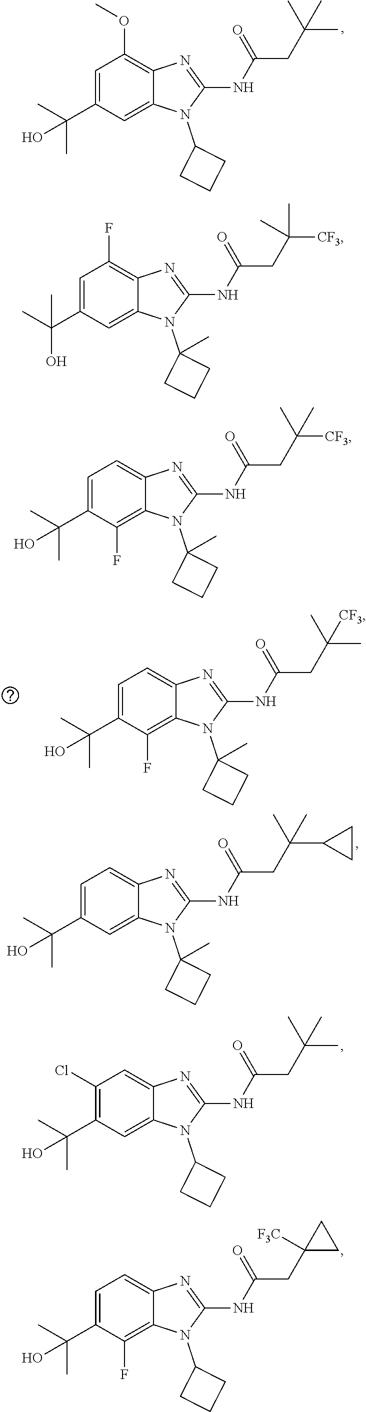

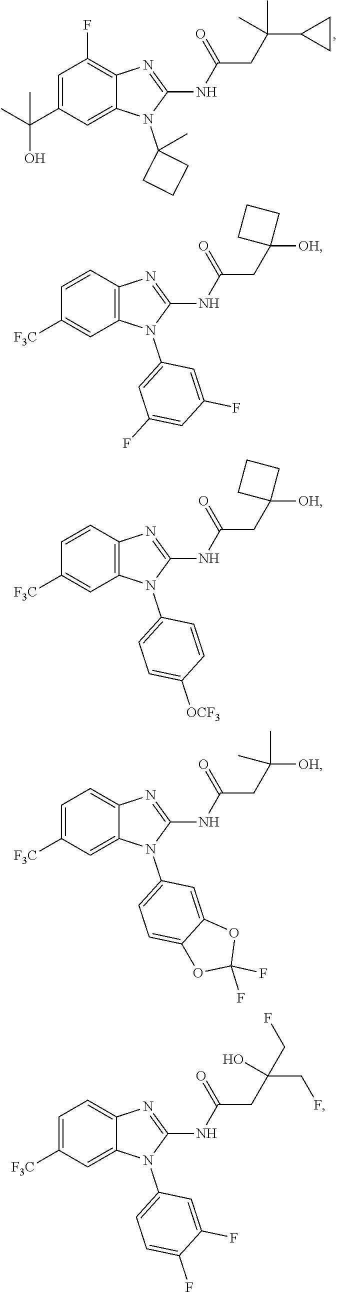

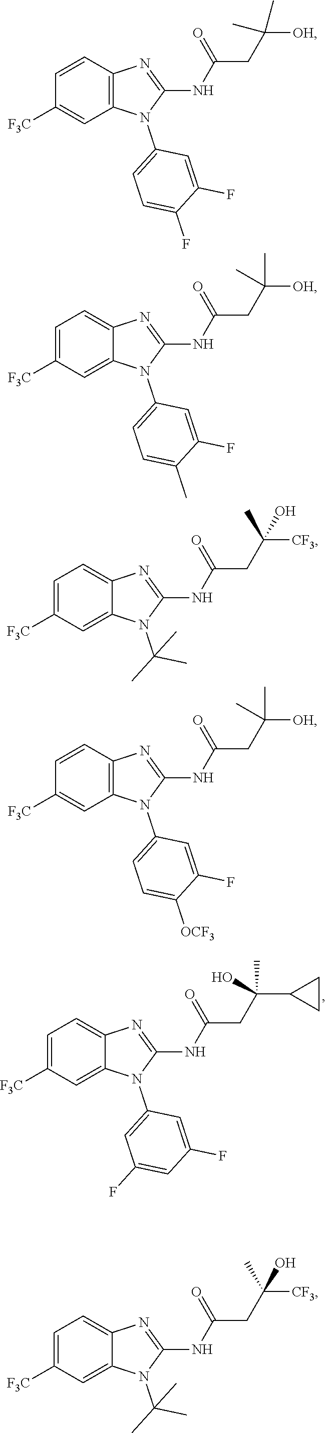

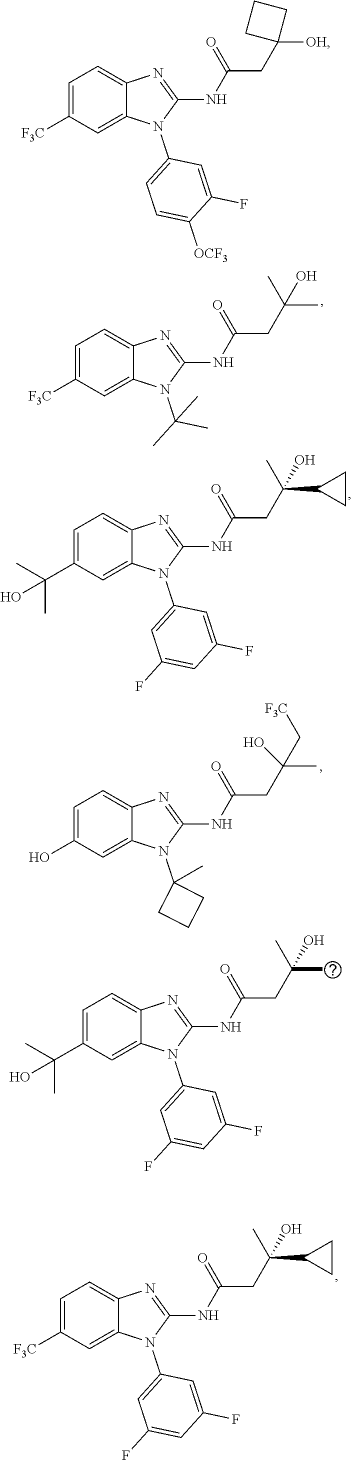

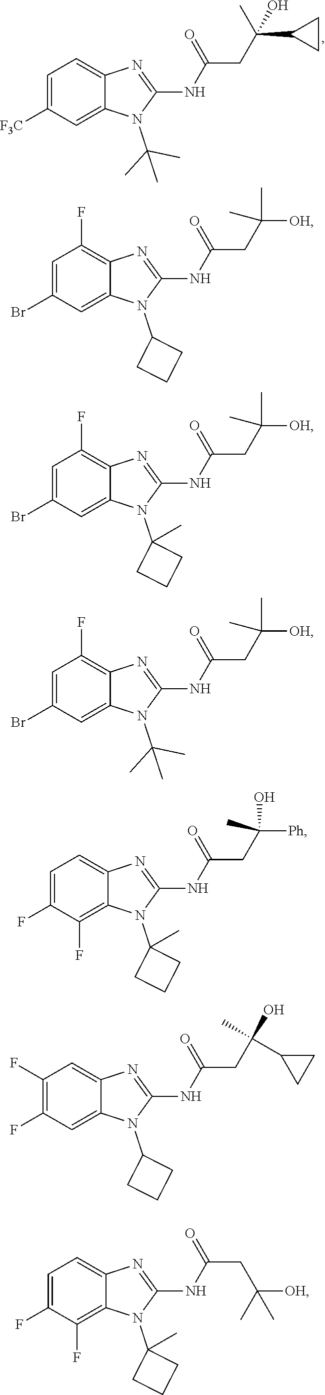

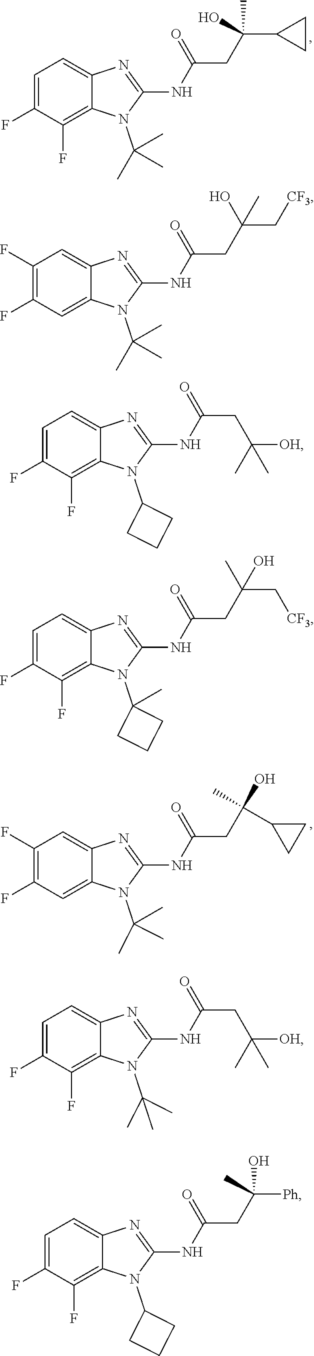

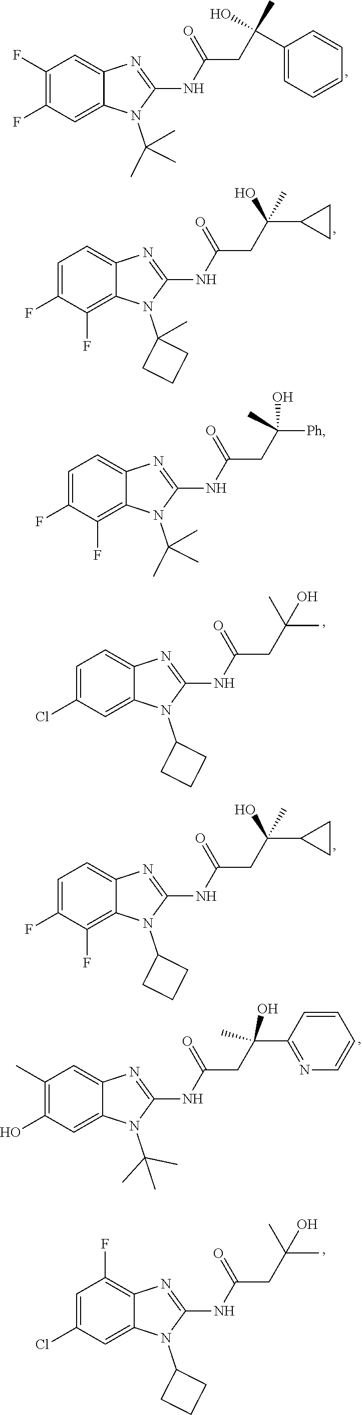

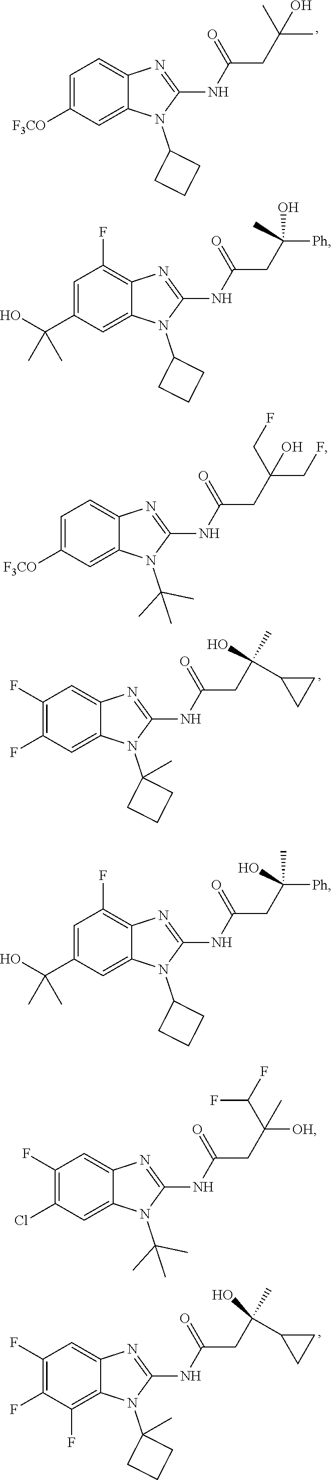

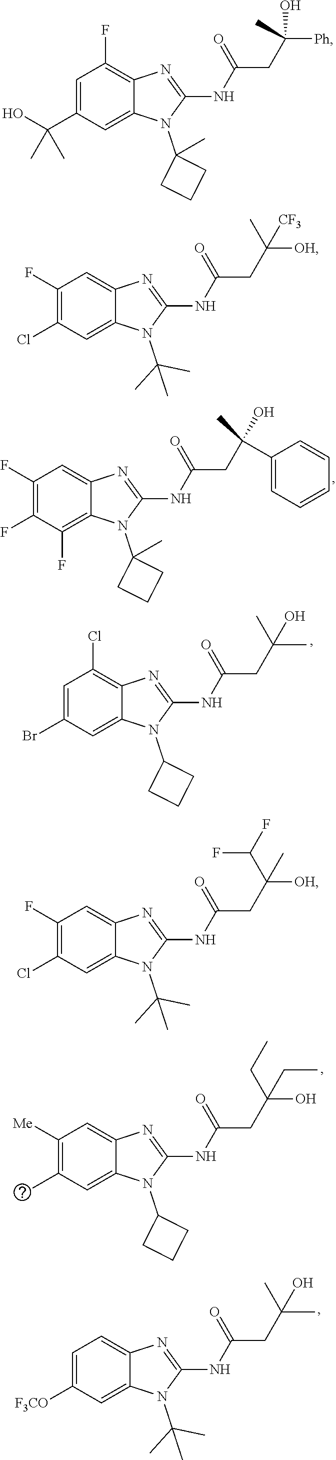

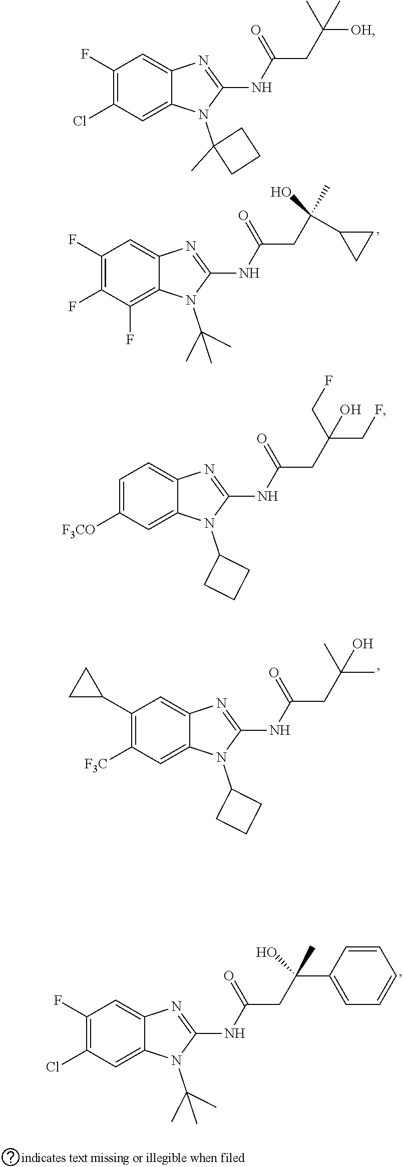

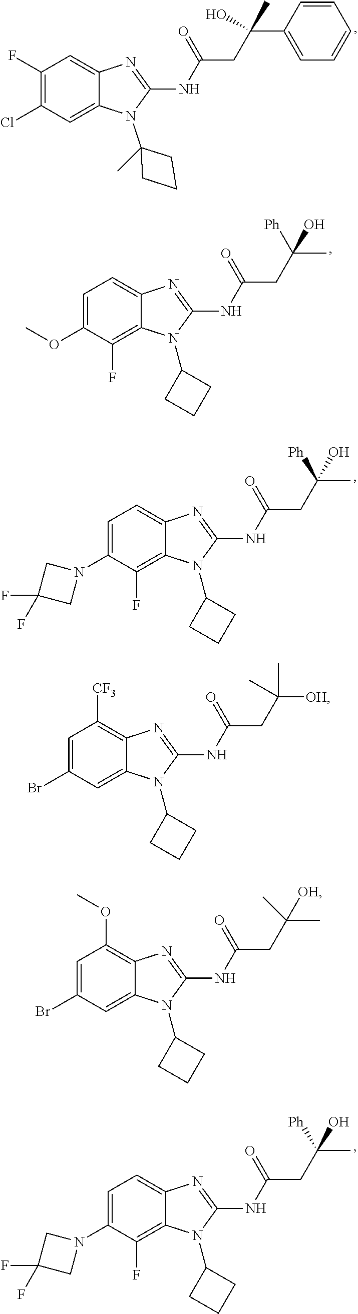 [SMILES: CC1(n2c(NC(=O)C[C@](C)(O)c3ccccc3)nc3cc(F)c(Cl)cc32)CCC1 ; COc1ccc2nc(NC(=O)C[C@](C)(O)c3ccccc3)n(C3CCC3)c2c1F ; C[C@@](O)(CC(=O)Nc1nc2ccc(N3CC(F)(F)C3)c(F)c2n1C1CCC1)c1ccccc1 ; CC(C)(O)CC(=O)Nc1nc2c(C(F)(F)F)cc(Br)cc2n1C1CCC1 ; COc1cc(Br)cc2c1nc(NC(=O)CC(C)(C)O)n2C1CCC1 ; C[C@](O)(CC(=O)Nc1nc2ccc(N3CC(F)(F)C3)c(F)c2n1C1CCC1)c1ccccc1]

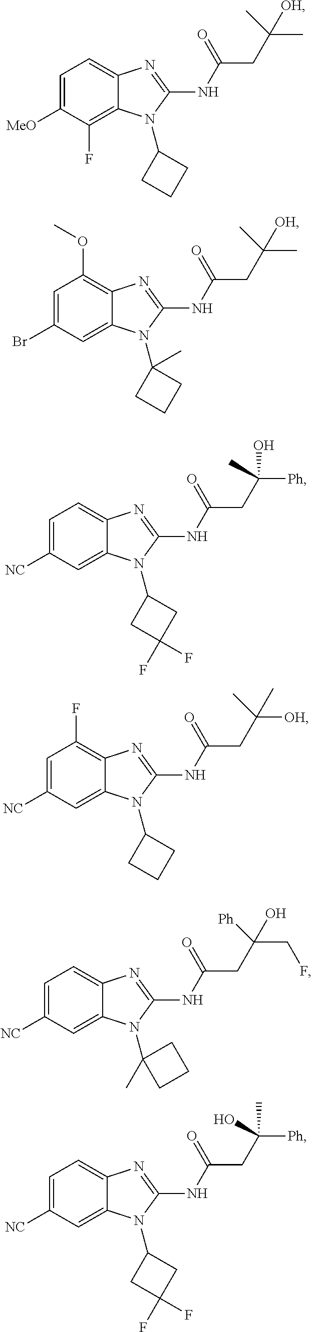 [SMILES: COc1ccc2nc(NC(=O)CC(C)(C)O)n(C3CCC3)c2c1F ; COc1cc(Br)cc2c1nc(NC(=O)CC(C)(C)O)n2C1(C)CCC1 ; C[C@](O)(CC(=O)Nc1nc2ccc(C#N)cc2n1C1CC(F)(F)C1)c1ccccc1 ; CC(C)(O)CC(=O)Nc1nc2c(F)cc(C#N)cc2n1C1CCC1 ; CC1(n2c(NC(=O)CC(O)(CF)c3ccccc3)nc3ccc(C#N)cc32)CCC1 ; C[C@@](O)(CC(=O)Nc1nc2ccc(C#N)cc2n1C1CC(F)(F)C1)c1ccccc1]

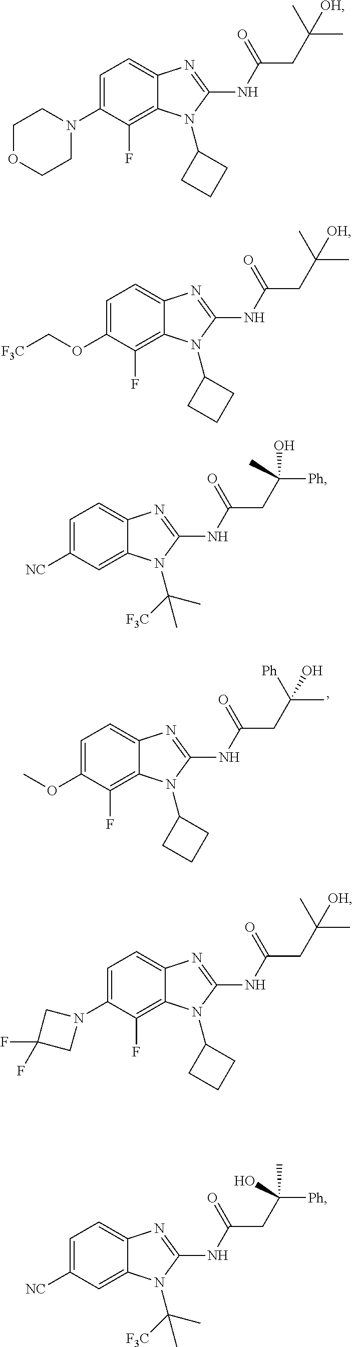

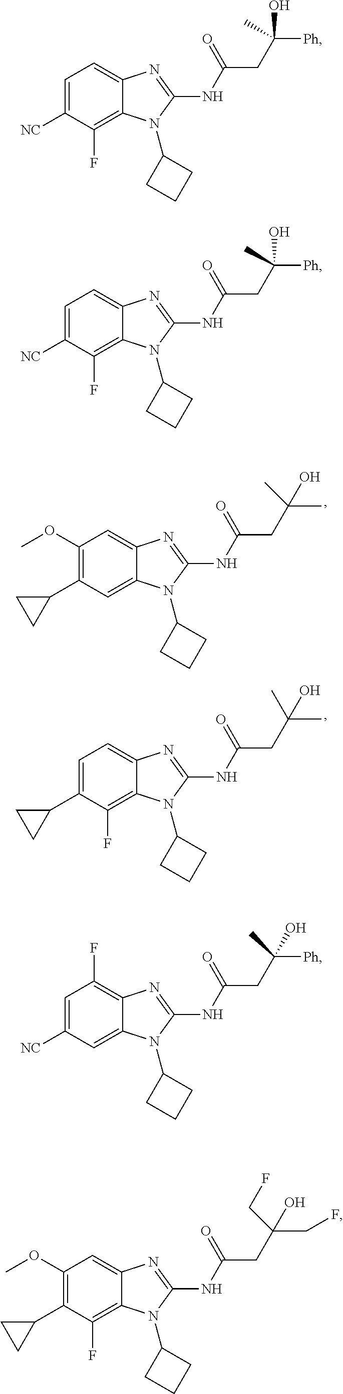 [SMILES: C[C@@](O)(CC(=O)Nc1nc2ccc(C#N)c(F)c2n1C1CCC1)c1ccccc1 ; C[C@](O)(CC(=O)Nc1nc2ccc(C#N)c(F)c2n1C1CCC1)c1ccccc1 ; COc1cc2nc(NC(=O)CC(C)(C)O)n(C3CCC3)c2cc1C1CC1 ; CC(C)(O)CC(=O)Nc1nc2ccc(C3CC3)c(F)c2n1C1CCC1 ; C[C@](O)(CC(=O)Nc1nc2c(F)cc(C#N)cc2n1C1CCC1)c1ccccc1 ; COc1cc2nc(NC(=O)CC(O)(CF)CF)n(C3CCC3)c2c(F)c1C1CC1]

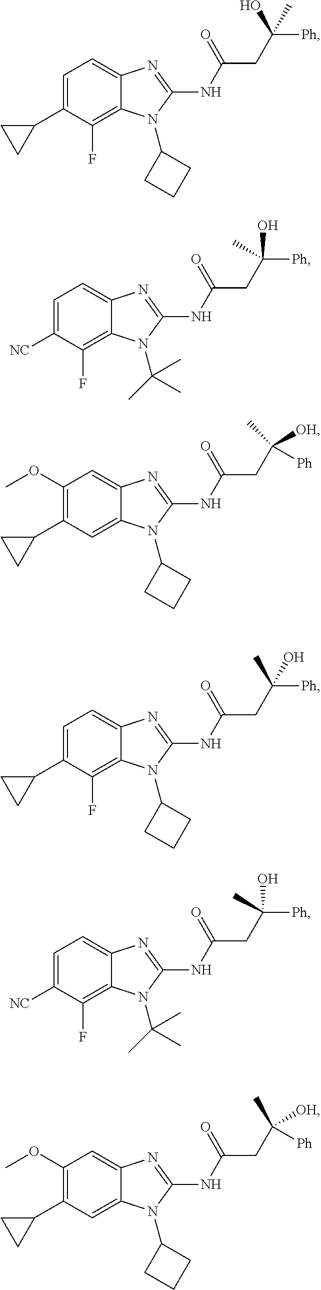 [SMILES: C[C@@](O)(CC(=O)Nc1nc2ccc(C3CC3)c(F)c2n1C1CCC1)c1ccccc1 ; CC(C)(C)n1c(NC(=O)C[C@@](C)(O)c2ccccc2)nc2ccc(C#N)c(F)c21 ; COc1cc2nc(NC(=O)C[C@@](C)(O)c3ccccc3)n(C3CCC3)c2cc1C1CC1 ; C[C@](O)(CC(=O)Nc1nc2ccc(C3CC3)c(F)c2n1C1CCC1)c1ccccc1 ; CC(C)(C)n1c(NC(=O)C[C@](C)(O)c2ccccc2)nc2ccc(C#N)c(F)c21 ; COc1cc2nc(NC(=O)C[C@](C)(O)c3ccccc3)n(C3CCC3)c2cc1C1CC1]

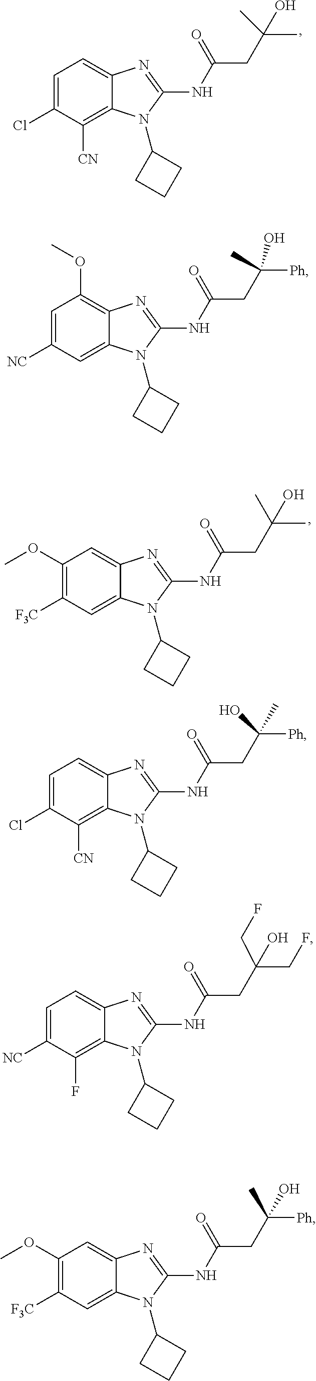

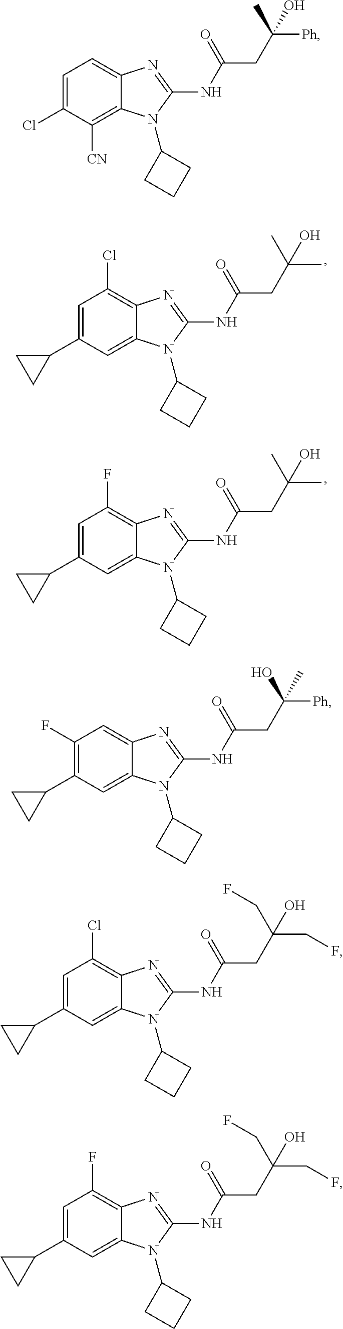 [SMILES: C[C@](O)(CC(=O)Nc1nc2ccc(Cl)c(C#N)c2n1C1CCC1)c1ccccc1 ; CC(C)(O)CC(=O)Nc1nc2c(Cl)cc(C3CC3)cc2n1C1CCC1 ; CC(C)(O)CC(=O)Nc1nc2c(F)cc(C3CC3)cc2n1C1CCC1 ; C[C@@](O)(CC(=O)Nc1nc2cc(F)c(C3CC3)cc2n1C1CCC1)c1ccccc1 ; O=C(CC(O)(CF)CF)Nc1nc2c(Cl)cc(C3CC3)cc2n1C1CCC1 ; O=C(CC(O)(CF)CF)Nc1nc2c(F)cc(C3CC3)cc2n1C1CCC1]

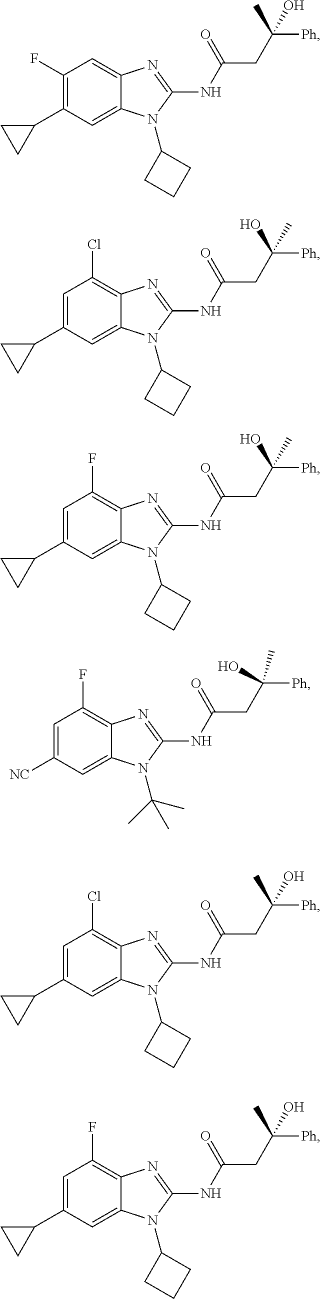 [SMILES: C[C@](O)(CC(=O)Nc1nc2cc(F)c(C3CC3)cc2n1C1CCC1)c1ccccc1 ; C[C@@](O)(CC(=O)Nc1nc2c(Cl)cc(C3CC3)cc2n1C1CCC1)c1ccccc1 ; C[C@@](O)(CC(=O)Nc1nc2c(F)cc(C3CC3)cc2n1C1CCC1)c1ccccc1 ; CC(C)(C)n1c(NC(=O)C[C@@](C)(O)c2ccccc2)nc2c(F)cc(C#N)cc21 ; C[C@](O)(CC(=O)Nc1nc2c(Cl)cc(C3CC3)cc2n1C1CCC1)c1ccccc1 ; C[C@](O)(CC(=O)Nc1nc2c(F)cc(C3CC3)cc2n1C1CCC1)c1ccccc1]

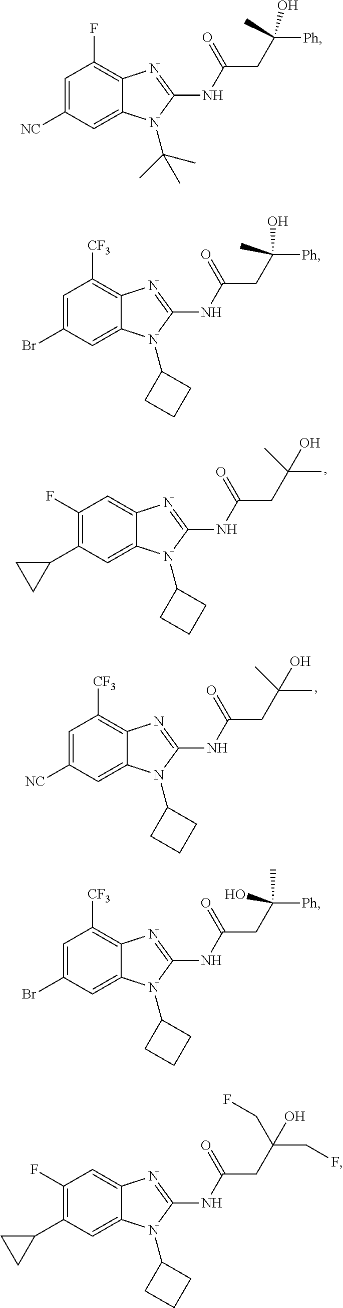 [SMILES: CC(C)(C)n1c(NC(=O)C[C@](C)(O)c2ccccc2)nc2c(F)cc(C#N)cc21 ; C[C@](O)(CC(=O)Nc1nc2c(C(F)(F)F)cc(Br)cc2n1C1CCC1)c1ccccc1 ; CC(C)(O)CC(=O)Nc1nc2cc(F)c(C3CC3)cc2n1C1CCC1 ; CC(C)(O)CC(=O)Nc1nc2c(C(F)(F)F)cc(C#N)cc2n1C1CCC1 ; C[C@@](O)(CC(=O)Nc1nc2c(C(F)(F)F)cc(Br)cc2n1C1CCC1)c1ccccc1 ; O=C(CC(O)(CF)CF)Nc1nc2cc(F)c(C3CC3)cc2n1C1CCC1]

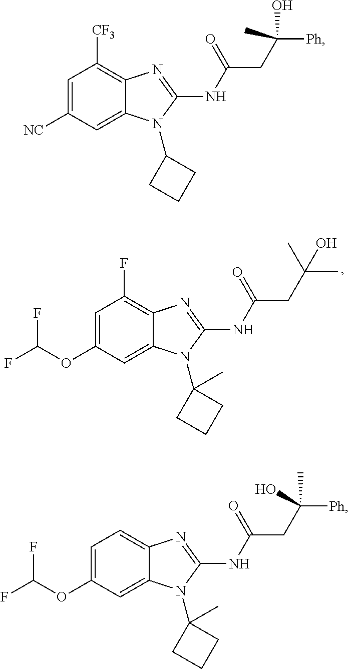 [SMILES: C[C@](O)(CC(=O)Nc1nc2c(C(F)(F)F)cc(C#N)cc2n1C1CCC1)c1ccccc1 ; CC(C)(O)CC(=O)Nc1nc2c(F)cc(OC(F)F)cc2n1C1(C)CCC1 ; CC1(n2c(NC(=O)C[C@@](C)(O)c3ccccc3)nc3ccc(OC(F)F)cc32)CCC1]

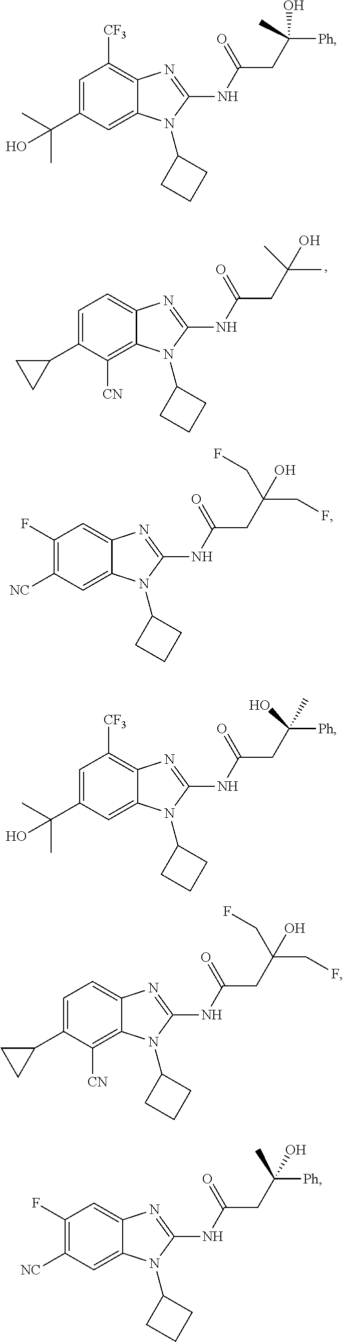

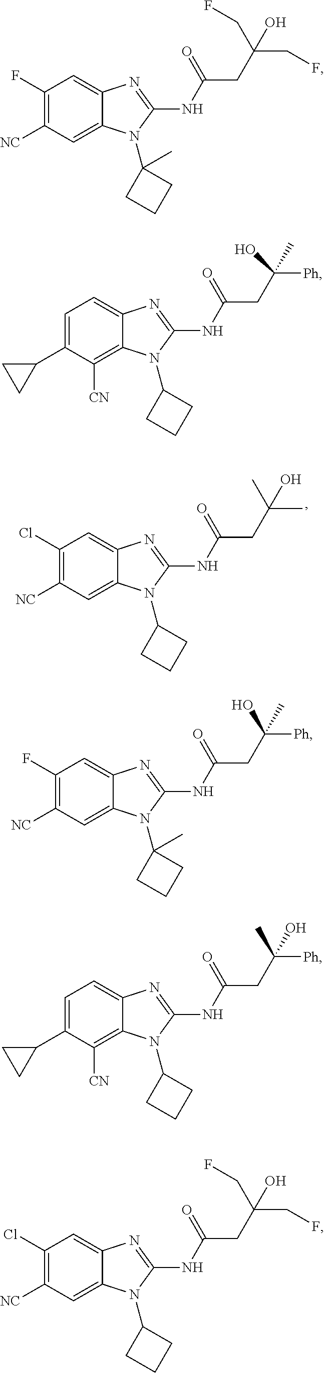 [SMILES: CC1(n2c(NC(=O)CC(O)(CF)CF)nc3cc(F)c(C#N)cc32)CCC1 ; C[C@@](O)(CC(=O)Nc1nc2ccc(C3CC3)c(C#N)c2n1C1CCC1)c1ccccc1 ; CC(C)(O)CC(=O)Nc1nc2cc(Cl)c(C#N)cc2n1C1CCC1 ; CC1(n2c(NC(=O)C[C@@](C)(O)c3ccccc3)nc3cc(F)c(C#N)cc32)CCC1 ; C[C@](O)(CC(=O)Nc1nc2ccc(C3CC3)c(C#N)c2n1C1CCC1)c1ccccc1 ; N#Cc1cc2c(cc1Cl)nc(NC(=O)CC(O)(CF)CF)n2C1CCC1]

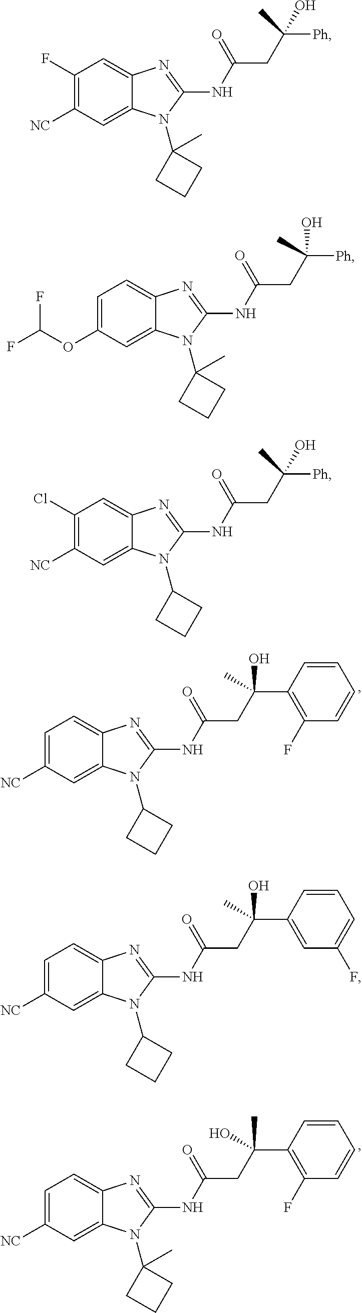 [SMILES: CC1(n2c(NC(=O)C[C@](C)(O)c3ccccc3)nc3cc(F)c(C#N)cc32)CCC1 ; CC1(n2c(NC(=O)C[C@](C)(O)c3ccccc3)nc3ccc(OC(F)F)cc32)CCC1 ; C[C@](O)(CC(=O)Nc1nc2cc(Cl)c(C#N)cc2n1C1CCC1)c1ccccc1 ; C[C@@](O)(CC(=O)Nc1nc2ccc(C#N)cc2n1C1CCC1)c1ccccc1F ; C[C@@](O)(CC(=O)Nc1nc2ccc(C#N)cc2n1C1CCC1)c1cccc(F)c1 ; CC1(n2c(NC(=O)C[C@](C)(O)c3ccccc3F)nc3ccc(C#N)cc32)CCC1]

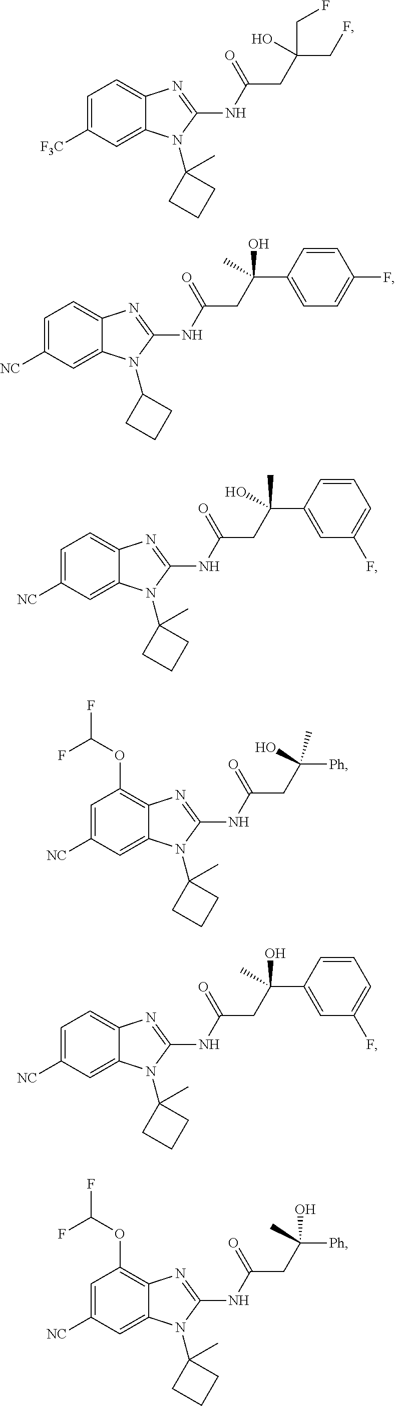 [SMILES: CC1(n2c(NC(=O)CC(O)(CF)CF)nc3ccc(C(F)(F)F)cc32)CCC1 ; C[C@@](O)(CC(=O)Nc1nc2ccc(C#N)cc2n1C1CCC1)c1ccc(F)cc1 ; CC1(n2c(NC(=O)C[C@](C)(O)c3cccc(F)c3)nc3ccc(C#N)cc32)CCC1 ; CC1(n2c(NC(=O)C[C@@](C)(O)c3ccccc3)nc3c(OC(F)F)cc(C#N)cc32)CCC1 ; CC1(n2c(NC(=O)C[C@@](C)(O)c3cccc(F)c3)nc3ccc(C#N)cc32)CCC1 ; CC1(n2c(NC(=O)C[C@](C)(O)c3ccccc3)nc3c(OC(F)F)cc(C#N)cc32)CCC1]

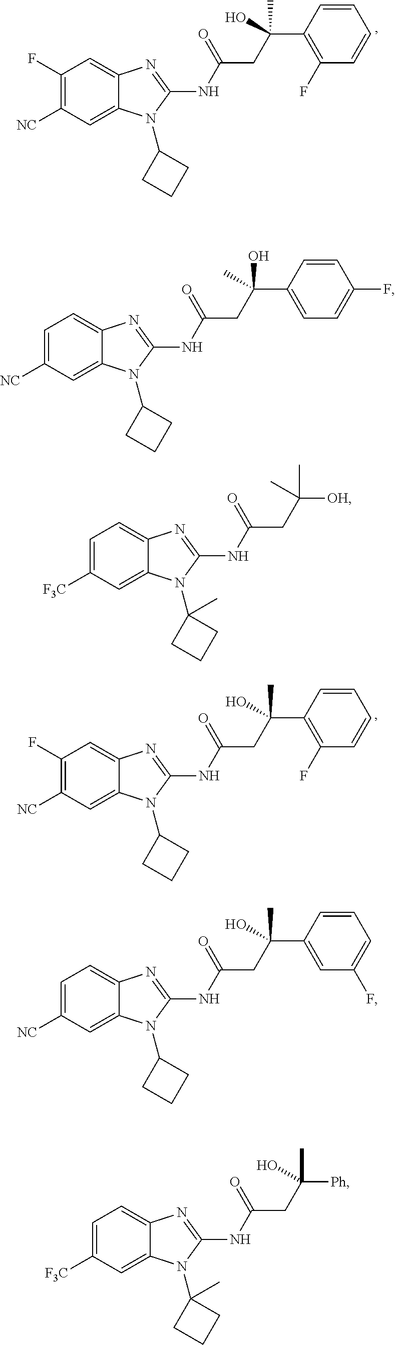 [SMILES: C[C@@](O)(CC(=O)Nc1nc2cc(F)c(C#N)cc2n1C1CCC1)c1ccccc1F ; C[C@@](O)(CC(=O)Nc1nc2ccc(C#N)cc2n1C1CCC1)c1ccc(F)cc1 ; CC(C)(O)CC(=O)Nc1nc2ccc(C(F)(F)F)cc2n1C1(C)CCC1 ; C[C@](O)(CC(=O)Nc1nc2cc(F)c(C#N)cc2n1C1CCC1)c1ccccc1F ; C[C@](O)(CC(=O)Nc1nc2ccc(C#N)cc2n1C1CCC1)c1cccc(F)c1 ; CC1(n2c(NC(=O)C[C@](C)(O)c3ccccc3)nc3ccc(C(F)(F)F)cc32)CCC1]

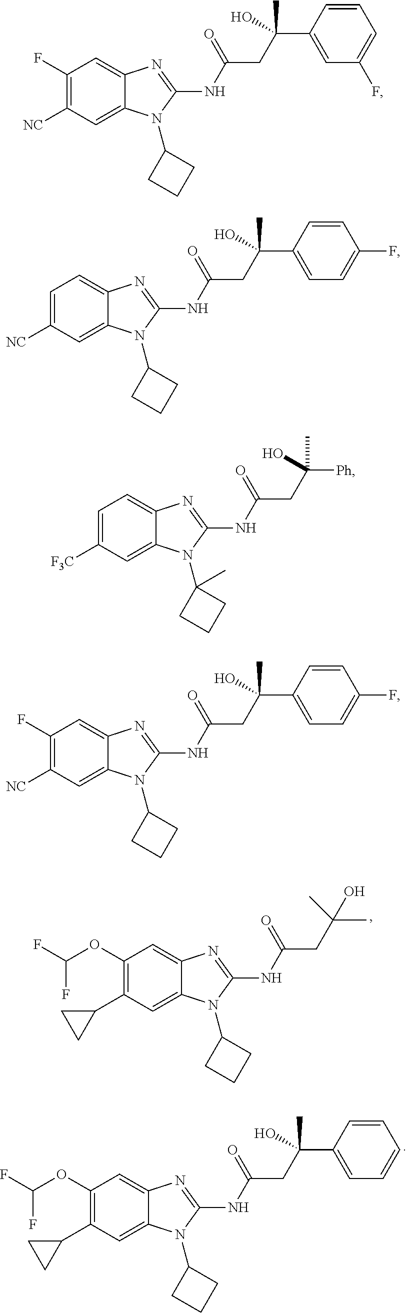 [SMILES: C[C@](O)(CC(=O)Nc1nc2cc(F)c(C#N)cc2n1C1CCC1)c1cccc(F)c1 ; C[C@](O)(CC(=O)Nc1nc2ccc(C#N)cc2n1C1CCC1)c1ccc(F)cc1 ; CC1(n2c(NC(=O)C[C@@](C)(O)c3ccccc3)nc3ccc(C(F)(F)F)cc32)CCC1 ; C[C@](O)(CC(=O)Nc1nc2cc(F)c(C#N)cc2n1C1CCC1)c1ccc(F)cc1 ; CC(C)(O)CC(=O)Nc1nc2cc(OC(F)F)c(C3CC3)cc2n1C1CCC1 ; C[C@](O)(CC(=O)Nc1nc2cc(OC(F)F)c(C3CC3)cc2n1C1CCC1)c1ccccc1]

### Formula 8

In an embodiment, the Kv7 channel activator may be selected from one of the following compounds according to Formula 8. Such compounds are described in U.S. Pat. No. 9,650,376, published on Feb. 4, 2014 and corresponding to U.S. application Ser. No. 14/776,271 filed Mar. 17, 2014; which is incorporated by reference in its entirety herein. In the case of any conflict of terminology in the context of Formula 8, this reference incorporated by reference herein controls.

In an embodiment, the Kv7 channel activator is a compound according to formula 8:

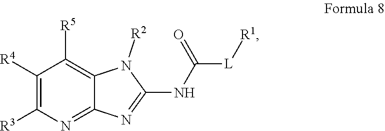

wherein, L is CH₂, CF₂, CHCH₃, CH₂CH₂, C₃H₆, CH₂O, C₂H₄O, or C₃H₆O with the O of CH₂O, C₂H₄O, or C₃H₆O bonded with R¹; wherein R¹ is optionally substituted C₁₋₂ alkyl, optionally substituted C₅₋₁₀ cycloalkyl, optionally substituted C₁₋₁₂-O-alkyl, optionally substituted C₆₋₁₀ aryl, optionally substituted C₆₋₁₀-O-aryl, or optionally substituted C₂₋₉ heterocyclyl, wherein the optional substituents of R¹ are independently R^{A}, F, Cl, CN, OR^{A}, CF₃, NR^{A}R^{B}, COR^{A}, CO₂R^{A}, OCOR^{A}, NR^{A}COR^{B}, CONR^{A}R^{B}, wherein R^{A} and R^{B} are independently H or C₁₋₁₂ alkyl; wherein R² is optionally substituted C₂₋₄ acyclic alkyl, optionally substituted cyclobutyl, optionally substituted C₆₋₁₀ aryl, or optionally substituted C₂₋₉ heterocyclyl, wherein the optional substituents of R² are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ alkylthio, or C₁₋₆ alkylsulfonyl; wherein R³, R⁴, and R⁵ are independently H, F, Cl, Br, I, CN, optionally substituted C₁₋₁₂ alkyl, optionally substituted C₁₋₁₂-O-alkyl, optionally substituted C₂₋₉ heterocyclyl, optionally substituted C₆₋₁₀ aryl, optionally substituted C₂₋₉—O-heterocyclyl, optionally substituted C₆₋₁₀ O-aryl, optionally substituted C₁₋₁₂ acylamino, optionally substituted C₁₋₁₂ aminoacyl, or optionally substituted C₁₋₁₂ aminoalkyl, wherein the optional substituents of R³, R⁴, or R⁵ are independently F, Cl, Br, I, CN, C₁₋₆ alkyl, C₁₋₆-O-alkyl, C₁₋₆ alkylamine, C1-6 aminoalkyl, C₁₋₆ aminoacyl, C1-6 acylamino, C1-6 alkylthio, or C1-6 alkylsulfonyl.

In further embodiments, R² is optionally substituted C₄H₉, optionally substituted cyclobutyl, optionally substituted C₆₋₁₀ aryl, or optionally substituted C₂₋₉ heterocyclyl.

In further embodiments, R² is optionally substituted C₄H₉, or optionally substituted cyclobutyl.

In further embodiments, R² is optionally substituted phenyl, or optionally substituted C₂₋₉ heterocyclyl.

In further embodiments, R¹ is optionally substituted phenyl.

In further embodiments, R¹ is optionally substituted phenyl.

In further embodiments, R¹ is optionally substituted cyclopentyl or optionally substituted cyclohexyl.

In further embodiments, R¹ is C₂₋₄-O-alkyl.

In further embodiments, R¹ is optionally substituted tetrahydrofuranyl or optionally substituted tetrahydro-2H-pyranyl.

In further embodiments, R¹ is selected from the group consisting of:

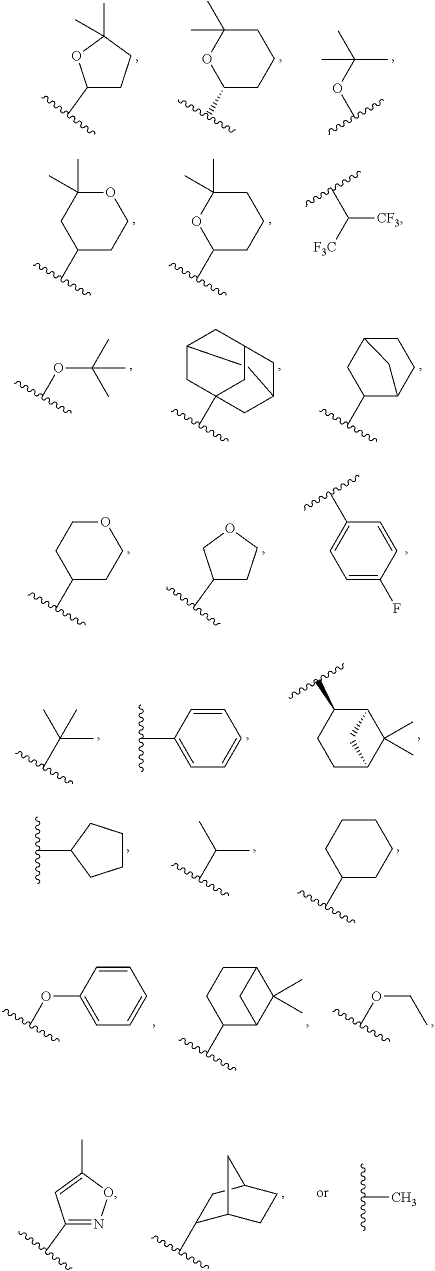

In further embodiments, R² is selected from the group consisting of:

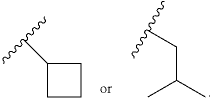

In further embodiments, R³ is CF₃, Cl, CN, OCH₃, or H.

In further embodiments, R¹ is selected from the group consisting of:

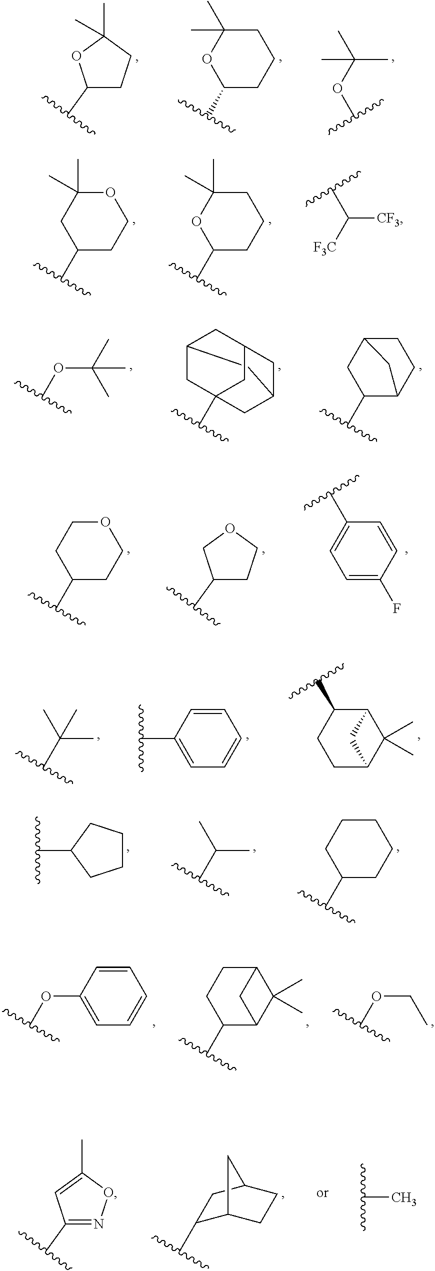

In further embodiments, R⁴ is CH₃, CF₃, Cl, or H.

In further embodiments, R² is selected from the group consisting of:

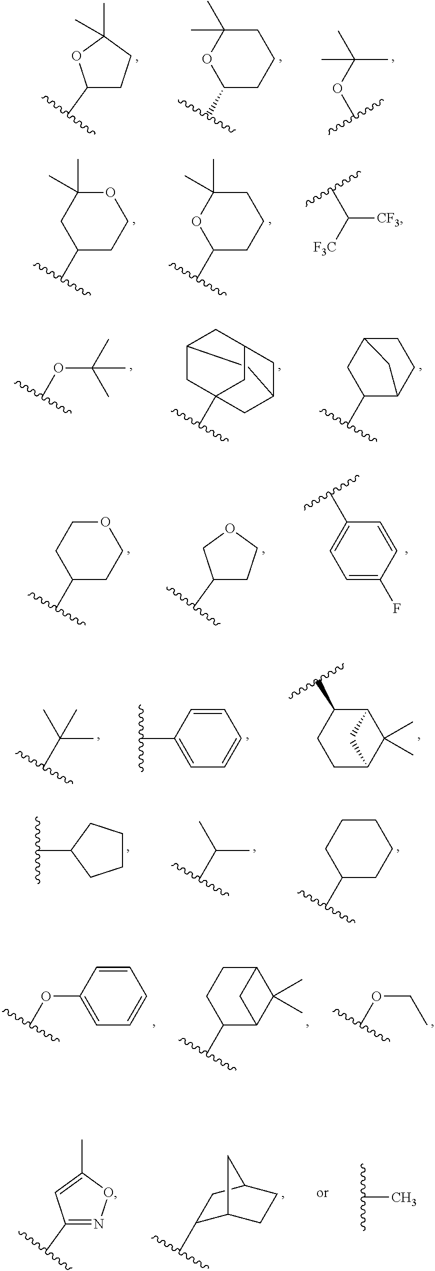

### TDP-43 Modulators

Amyotrophic lateral sclerosis (ALS) is a neurological disorder in which the upper and lower motor neurons progressively degenerate (Asakawa K, Handa H, Kawakami K. Optogenetic modulation of TDP-43 oligomerization accelerates ALS-related pathologies in the spinal motor neurons. Nat Commun. 2020 Feb. 21; 11(1): 1004. doi: 10.1038/s41467-020-14815-x. PMID: 32081878; PMCID: PMC7035286.). The degradation of motor neurons leads to muscular atrophy and eventually paralysis. In many individuals afflicted with ALS, Trans-activation response element (TAR) DNA-binding protein 43 (TDP-43), a heterogeneous nuclear ribonucleoprotein, is incorrectly localized to the cytoplasm. In the cytoplasm is forms aggregates associated with degenerating motor neurons. TDP-43 aggregates are present in nearly all cases of sporadic ALS. Moreover, a proportion of familial ALS is associated with mutations in the TARDBP gene encoding TDP-43. Modulation of TDP-43 in combination with the metal channel activator may therefore provide a promising approach to treat neurodegenerative disease, such as ALS.

Examples of TDP-43 modulators are disclosed in Formulas 200-206, and in the corresponding references applications. Such TDP-43 modulators are advantageously useful in combination therapies with Kv7 modulators as described herein.

### Formula 200

In an embodiment, the TDP-43 modulator may be selected from one of the following compounds according to Formula 200. Such compounds are described in International Publication No. WO2021035101A1, published Feb. 25, 2021, and corresponding to International Application No. PCT/US2020/047287 filed Aug. 21, 2020; which is incorporated by reference in its entirety herein. In the case of any conflict of terminology in the context of Formula 200, these references incorporated by reference herein control.

In an embodiment, the TDP-43 modulator is a compound according to Formula 200:

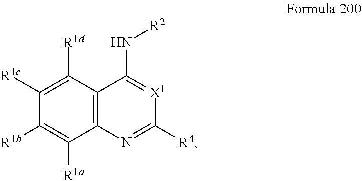

an enantiomer, a diastereomer, a hydrate, a solvate, a pharmaceutically acceptable salt, an isotopic analog, a prodrug or a complex thereof, wherein: X¹ is selected from the group consisting of nitrogen and CH; R^{1a} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; R^{1b} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; R^{1c} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; and R^{1d} is selected from the group consisting of hydrogen, halogen, C₁₋₂₀ linear alkyl, C₃₋₂₀ branched alkyl, C₁₋₂₀ linear heteroalkyl, C₃₋₂₀ branched heteroalkyl, each of which except hydrogen and halogen are optionally substituted; R² is a substituted or unsubstituted C₁-C₂₀ linear, branched, or cyclic organic group including at least one nitrogen; and R⁴ is a hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C6-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In further embodiments, X¹ is selected from the group consisting of nitrogen and CH; R^{1a} is selected from the group consisting of hydrogen, halogen, CF₃, OCF₃, C₁₋₄ linear alkyl, C₃₋₄ branched alkyl, (C₂₋₈ dialkylamino) (C₂₋₄ alkyl), (C₃₋₆ alkyleneamino) (C₂₋₄ alkyl), C₁₋₄ linear alkoxy, and C₃₋₄ branched alkoxy; R^{1b} is selected from the group consisting of hydrogen, halogen, CF₃, OCF₃, C₁₋₄ linear alkyl, C₃₋₄ branched alkyl, (C₂₋₈ dialkylamino) (C₂₋₄ alkyl), (C₃₋₆ alkyleneamino) (C₂₋₄ alkyl), C₁₋₄ linear alkoxy, and C₃₋₄ branched alkoxy; R^{1c} is selected from the group consisting of hydrogen, halogen, CF₃, OCF₃, C₁₋₄ linear alkyl, C₃₋₄ branched alkyl, (C₂₋₈ dialkylamino) (C₂₋₄ alkyl), (C₃₋₆ alkyleneamino) (C₂₋₄ alkyl), C₁₋₄ linear alkoxy, and C₃₋₄ branched alkoxy; R^{1d} is selected from the group consisting of hydrogen, halogen, CF₃, OCF₃, C₁₋₄ linear alkyl, C₃-4 branched alkyl, (C2-8 dialkylamino) (C2-4 alkyl), (C3-6 alkyleneamino) (C2-4 alkyl), C₁₋₄ linear alkoxy, and C34 branched alkoxy; wherein any two selected from the group consisting of R^{1a}, R^{1b}, R^{1c}, and R^{1d} are optionally connected to form a ring; R² is selected from the group consisting of —(CH₂)ₙ—NR⁵R⁶, —(CH₂)ₙC(0)-NR⁵R⁶,

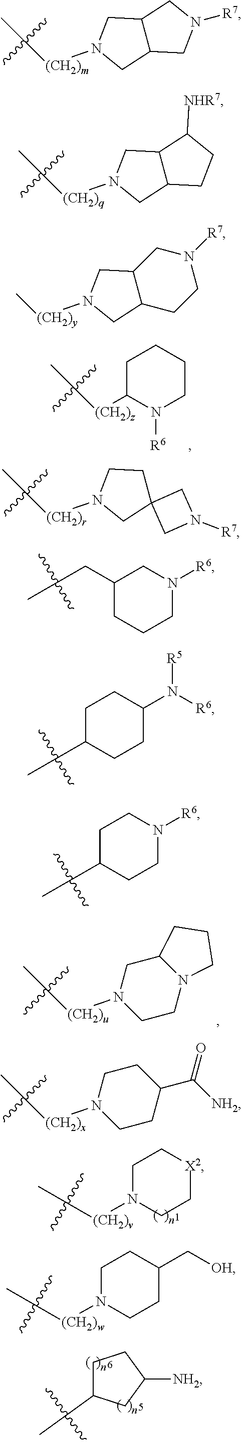

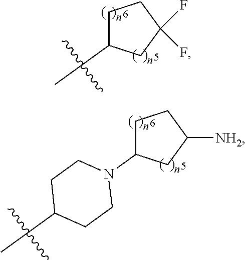 [SMILES: *C1CCC(F)(F)C1 ; *C1CCN(C2CCC(N)C2)CC1]

n¹ is 1 or 2; R⁴ is hydrogen; CF₃; a five-membered monocyclic heteroaryl ring comprising at least one heteroatom selected from the group consisting from O, N, and S that is optionally substituted with up to 2 groups selected from C₁₋₄ linear alkyl, C3-4 branched alkyl, C₁₋₄ linear alkoxy, C3-4 branched alkoxy, CF3, CF3O, halogen,

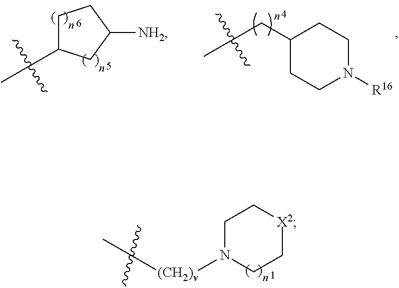

a five-membered monocyclic heteroaryl ring comprising at least one heteroatom selected from the group consisting from O, N, and S;

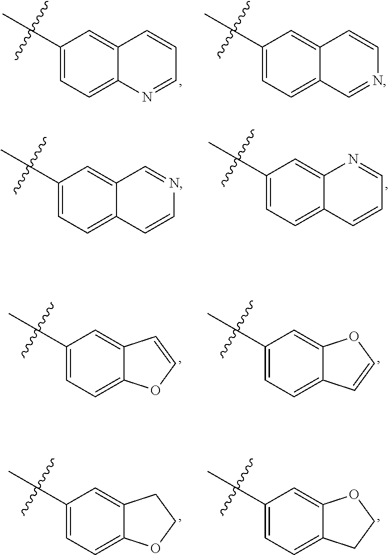

a phenyl ring that is optionally substituted with up to 2 groups selected from C₁₋₄ linear alkyl, C₃₋₄ branched alkyl, C₁₋₄ linear alkoxy, C₃₋₄ branched alkoxy, C₃₋₆ cycloalkyl, C₃₋₆ cycloalkoxy, CF₃, CF₃O, halogen, NR^{3a}R^{3b}, SO₂NR^{3a}R^{3b}, NHSO₂R^{3a},

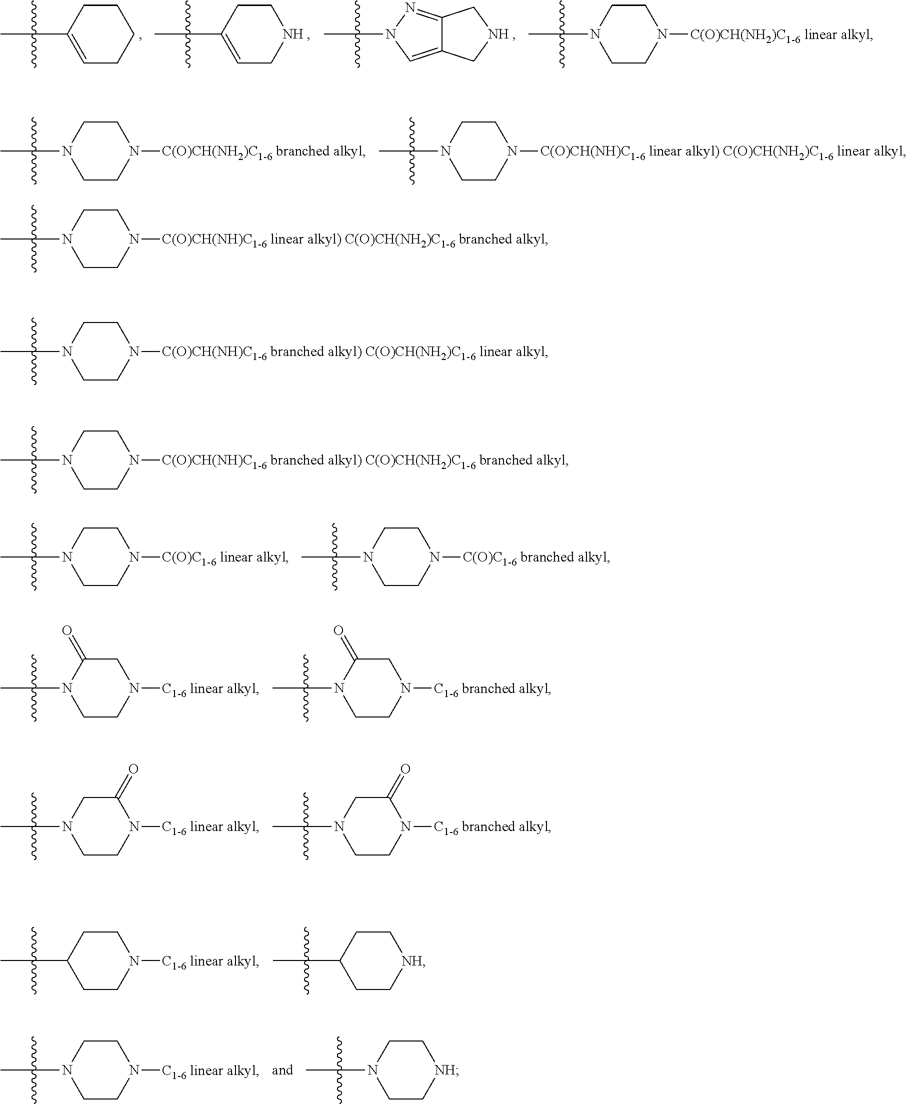

wherein, R^{3a} is at each occurrence independently selected from the group consisting of C₁₋₄ linear alkyl and C₃₋₄ branched alkyl; R^{3b} is at each occurrence independently selected from the group consisting of C₁₋₄ linear alkyl and C₃₋₄ branched alkyl; R⁵ is selected from the group consisting of hydrogen, C₁₋₄ linear alkyl, C3-4 branched alkyl, —CH2—(C1-6 cycloalkyl), C(0)C1-6 linear alkyl, C(0)C₃₋₆ branched alkyl, C(O)CH (NH₂)C₁₋₆ linear alkyl, C(O)CH (NH₂)C₃₋₆ branched alkyl,

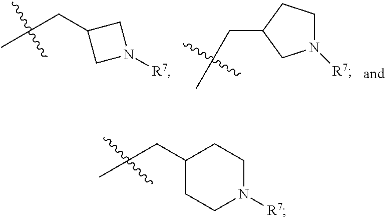

R⁶ is selected from the group consisting of C₁₋₄ linear alkyl, C3-4 branched alkyl,

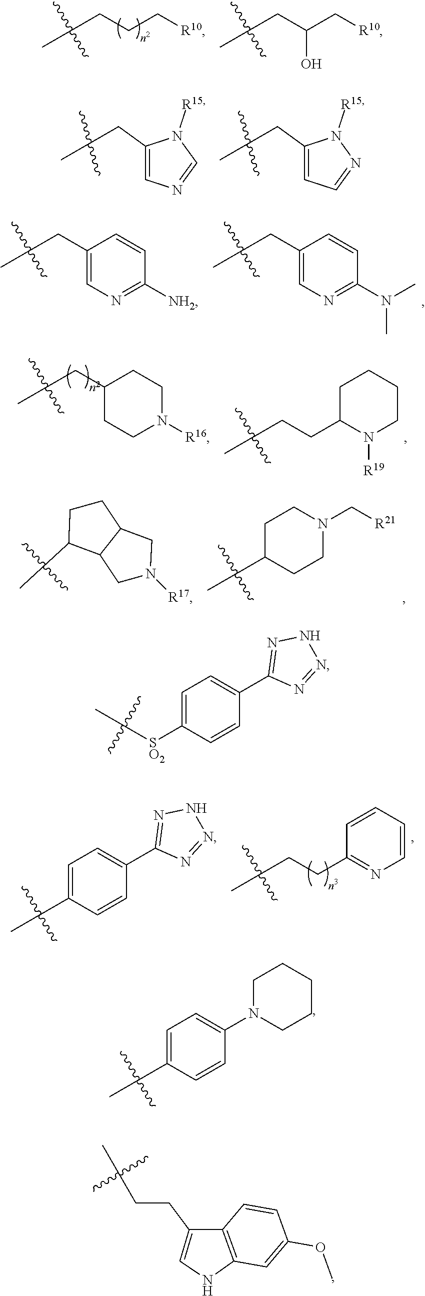

and an optionally and an optionally substituted benzyl group wherein the optionally substituted benzyl group is substituted with 0 to 2 groups selected from the group consisting of halogen, —CN, —NO₂, —OH, —NH₂, Ci-6 alkyl, C₃₋₇ branched alkyl, Ci-6 linear haloalkyl, C₃₋₇ branched haloalkyl, Ci-6 linear alkoxy, C₃₋₇ branched alkoxy, C1-6 linear haloalkoxy, C₃₋₇ branched haloalkoxy, C₃₋₇ cycloalkyl, aryl, heterocycle, and heteroaryl, provided that when R² is

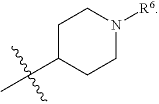

R⁶ is not hydrogen, C₁₋₄ linear alkyl, C3-4 branched alkyl, or benzyl; n² is 1 or 2; n³ is 0 or 1; n⁴ is 0 or 1; n⁵ is 0, 1, or 2; n⁶ is 0 or 1; R⁷ is selected from the group consisting of hydrogen and C(0)OR⁸; R⁸ is selected from the group consisting of C₁₋₄ linear alkyl and C₃₋₄ branched alkyl; X² is selected from the group consisting of a single bond, oxygen, CH₂, CHOH, and NR⁹; R⁹ is C₁₋₄ linear alkyl that is optionally substituted with an NH₂; R¹⁰ is selected from the group consisting of OH, OR¹¹, NR¹²R¹³, NHSO₂R²², and

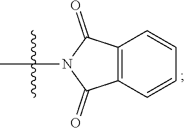 [SMILES: *N1C(=O)c2ccccc2C1=O]

R¹¹ is selected from the group consisting of C₁₋₄ linear alkyl and C₃₋₄ branched alkyl; R¹² is selected from the group consisting of hydrogen, C₁₋₄ linear alkyl, C₃₋₄ branched alkyl, C₁₋₄ linear fluoroalkyl, C(O)R¹⁴; R¹³ is selected from the group consisting of hydrogen, C₁₋₄ linear alkyl, C₃₋₄ branched alkyl, and heteroaryl; R¹² and R¹³ are optionally taken together with the atoms to which they are connected to form a ring with 3 to 6 atoms; R¹⁴ is selected from the group consisting of C₁₋₄ linear alkyl C₃₋₄ branched alkyl, and

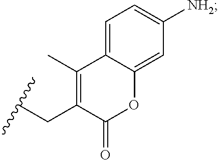 [SMILES: *Cc1c(C)c2ccc(N)cc2oc1=O]

R¹⁵ is selected from the group consisting of C₁₋₄ linear alkyl and C₃₋₄ branched alkyl; R¹⁶ is selected from the group consisting of hydrogen, C₁₋₄ linear alkyl, C₃₋₄ branched alkyl, CH₂—(C₁₋₆ cycloalkyl), and C(0)R¹⁸; R¹⁷ is selected from the group consisting of hydrogen, benzyl and C(0)R¹⁸; R¹⁸ is selected from the group consisting of C₁₋₄ linear alkyl and C₃₋₄ branched alkyl; R¹⁹ is selected from the group consisting of hydrogen and C(0)R²⁰; R²⁰ is selected from the group consisting of C₁₋₄ linear alkyl and C₃₋₄ branched alkyl; R²¹ is a benzene ring that is optionally substituted with 0 to 2 groups selected from the group consisting of halogen, —CN, —NO₂, —OH, —NH₂, C₁₋₆ alkyl, C₃₋₇ branched alkyl, C₁₋₆ linear haloalkyl, C₃₋₇ branched haloalkyl, C₁₋₆ linear alkoxy, C₃₋₇ branched alkoxy, C₁₋₆ linear haloalkoxy, C₃₋₇ branched haloalkoxy, C₃₋₇ cycloalkyl, aryl, heterocycle, and heteroaryl; R²² is selected from the group consisting of C₁₋₄ linear alkyl, C34 branched alkyl,

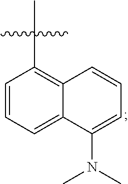 [SMILES: *c1cccc2c(N(C)C)cccc12]

n is 2, 3, or 4; m is 2, 3, or 4; q is 2, 3, or 4; y is 2, 3, or 4; u is 2, 3, or 4; v is 2, 3, or 4; w is 2, 3, or 4; z is 1, 2 or 3; r is 2, 3, or 4; and x is 2, 3, or 4.

In further embodiments, the compound is:

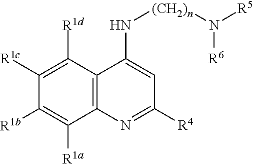

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, R⁵, and R⁶ are defined as above.

In further embodiments, the compound is:

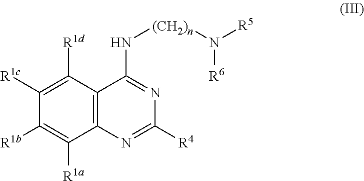

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, R⁵, and R⁶ are defined as above.

In further embodiments, the compound is:

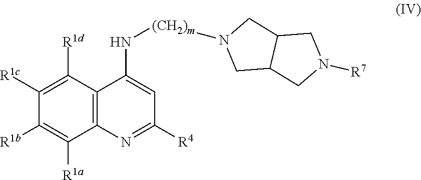

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, R⁶, and R⁷ are defined as above.

In further embodiments, the compound is:

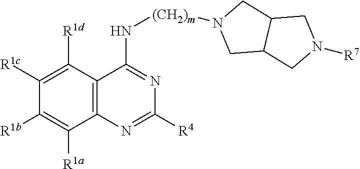

an enantiomer, a diastereomer, a hydrate, a solvate, a pharmaceutically acceptable salt, an isotopic analog, a prodrug, or a complex thereof, wherein m, R^{1a}, R^{1b}, R^{1c}, R^{1d}, R⁴, R⁶, and R⁷ are defined as above.

In further embodiments, the compound is:

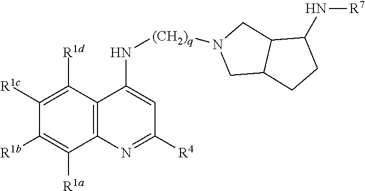

an enantiomer, a diastereomer, a hydrate, a solvate, a pharmaceutically acceptable salt, an isotopic analog, a prodrug, or a complex thereof, wherein q, R^{1a}, R^{1b}, R^{1c}, R^{1d}, R⁴, R⁶, and R⁷ are defined as above.

In further embodiments, the compound is:

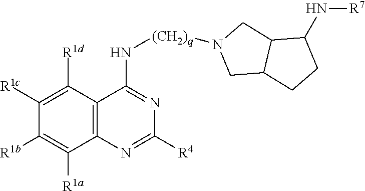

an enantiomer, a diastereomer, a hydrate, a solvate, a pharmaceutically acceptable salt, an isotopic analog, a prodrug, or a complex thereof, wherein q, R^{1a}, R^{1b}, R^{1c}, R^{1d}, R⁴, R⁶, and R⁷ are defined as above.

In further embodiments, the compound is:

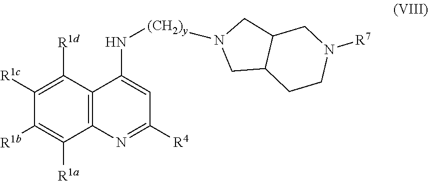

an enantiomer, a diastereomer, a hydrate, a solvate, a pharmaceutically acceptable salt, an isotopic analog, a prodrug, or a complex thereof, wherein y, R^{1a}, R^{1b}, R^{1c}, R^{1d}, R⁴, R⁶, and R⁷ are defined as above.

In further embodiments, the compound is:

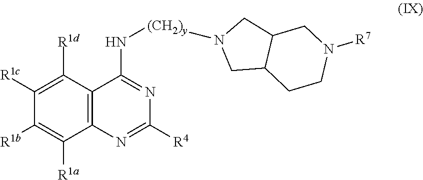

an enantiomer, a diastereomer, a hydrate, a solvate, a pharmaceutically acceptable salt, an isotopic analog, a prodrug, or a complex thereof, wherein y, R¹, R^{1b}, R^{1c}, R^{1d}, R⁴, R⁶, and R⁷ are defined as above.

In further embodiments, the compound is:

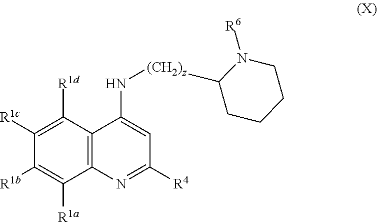

an enantiomer, a diastereomer, a hydrate, a solvate, a pharmaceutically acceptable salt, an isotopic analog, a prodrug, or a complex thereof, wherein z, R^{1a}, R^{1b}, R^{1c}, R^{1d}, R⁴, and R⁶ are defined as above.

In further embodiments, the compound is:

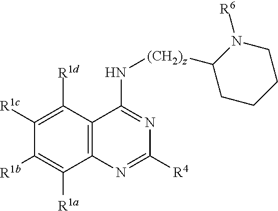

an enantiomer, a diastereomer, a hydrate, a solvate, a pharmaceutically acceptable salt, an isotopic analog, a prodrug, or a complex thereof, wherein z, R^{1a}, R^{1b}, R^{1c}, R^{1d}, R⁴, and R⁶ are defined as above.

In further embodiments, the compound is:

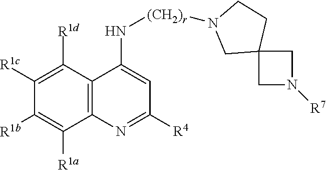

an enantiomer, a diastereomer, a hydrate, a solvate, a pharmaceutically acceptable salt, an isotopic analog, a prodrug, or a complex thereof, wherein r, R^{1a}, R^{1b}, R^{1c}, R^{1d}, R⁴, and R⁷ are defined as above.

In further embodiments, the compound is:

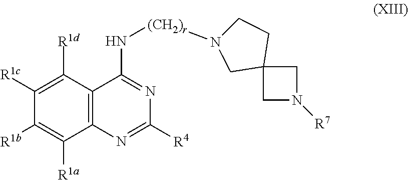

an enantiomer, a diastereomer, a hydrate, a solvate, a pharmaceutically acceptable salt, an isotopic analog, a prodrug, or a complex thereof, wherein r, R^{1a}, R^{1b}, R^{1c}, R^{1d}, R⁴, and R⁷ are defined as above.

In further embodiments, the compound is:

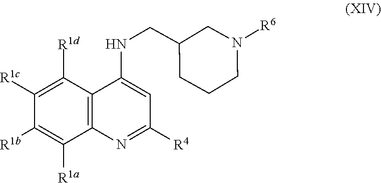

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and R⁶ are defined as above.

In further embodiments, the compound is:

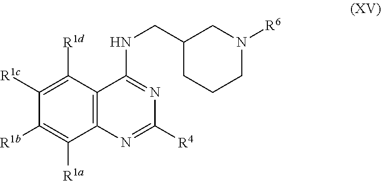

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and R⁶ are defined as above.

In further embodiments, the compound is:

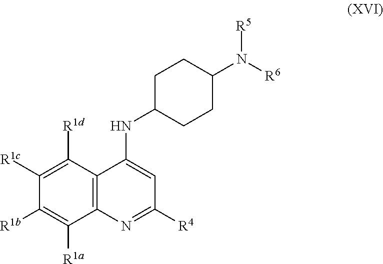

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, R⁵, and R⁶ are defined as above.

In further embodiments, the compound is:

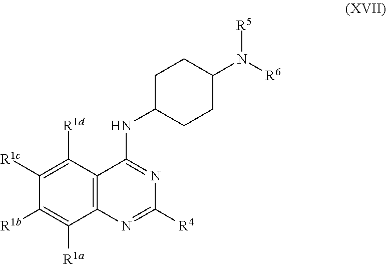

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, R⁵, and R⁶ are defined as above.

In further embodiments, the compound is:

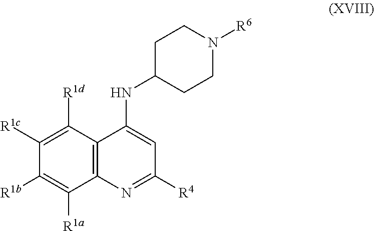

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, R⁵, and R⁶ are defined as above.

In further embodiments, the compound is:

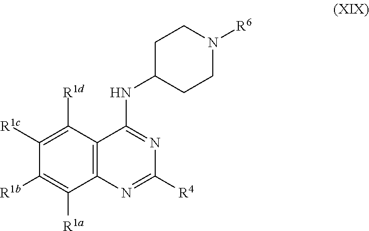

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴ and R⁶ are defined as above.

In further embodiments, the compound is:

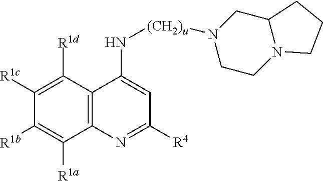

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and u are defined as above.

In further embodiments, the compound is:

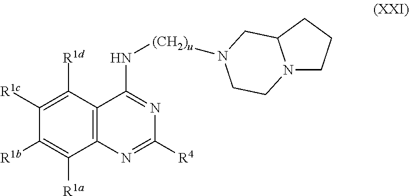

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and u are defined as above.

In further embodiments, the compound is:

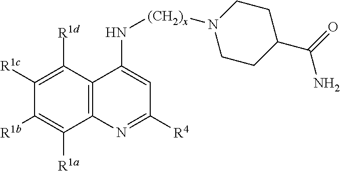

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴ and x are defined as above.

In further embodiments, the compound is:

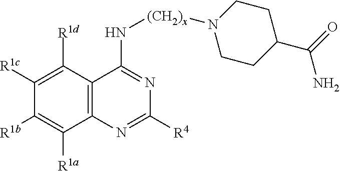

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and x are defined as above.

In further embodiments, the compound is:

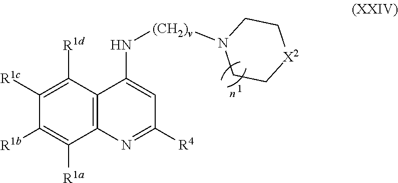

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, v, X², and n¹ are defined as above.

In further embodiments, the compound is:

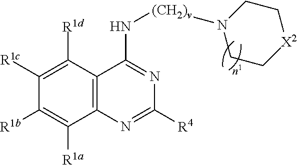

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴ v, X², and n¹ are defined as above.

In further embodiments, the compound is:

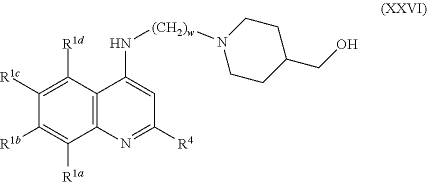

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and w are defined as above.

In further embodiments, the compound is:

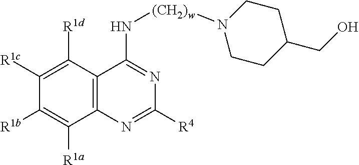

an enantiomer, a diastereomer, a hydrate, a solvate, a pharmaceutically acceptable salt, an isotopic analog, a prodrug, or a complex thereof, wherein R^{1a}, R^{1b}, R^{1c}, R^{1d}, R⁴, and w are defined as above.

In further embodiments, the TDP-43 modulator is selected from the group consisting of: N¹-(3-((2-(4-Methoxyphenyl)quinolin-4-yl)amino)propyl)-N¹,N³,N³-trimethylpropane-1,3-diamine; 2-(4-Methoxyphenyl)-N-(3-{methyl[3-(methylamino)propyl]-amino}propyl)quinolin-4-amine; N¹-(3-Aminopropyl)-N³-(2-(4-methoxyphenyl)quinolin-4-yl)-N¹-methylpropane-1,3-diamine; N¹-(2-(4-Methoxyphenyl)quinolin-4-yl)-N³-(3-(piperidin-1-yl)propyl) propane-1,3-diamine; N¹-(2-(4-methoxyphenyl)quinolin-4-yl)-N³-methyl-N³-(3-(piperidin-I-yl)propyl) propane-1,3-diamine; tert-ButylN-{3-[(3-{[2-(4-methoxyphenyl)quinolin-4yl]amino}propyl)amino]propyl}carbamate; N¹-(3-aminopropyl)-N³-(5-fluoro-2-(4-methoxyphenyl)quinolin-4-yl)-N¹-methylpropane-1,3-diamine: N-{3-[(3-Aminopropyl)amino]propyl}-2-(4-methoxyphenyl)quinolin-4-amine; N¹-(3-Aminopropyl)-N³-(6-methoxy-2-(4-methoxyphenyl)quinolin-4-yl)-N¹-methylpropane-1,3-diamine; N¹-(3-Aminopropyl)-N³-(2-(2-methyl-4-methoxyphenyl)quinolin-4-yl)-N¹-methylpropane-1,3-diamine; N¹-(3-Aminopropyl)-N³-(6,7-dimethoxy-2-(4-methoxyphenyl)quinolin-4-yl)-N¹-methylpropane-1,3-diamine; N¹-(3-((2-(4-Methoxyphenyl)quinolin-4-yl)amino)propyl)-N¹,N⁴-dimethylbutane-1,4-diamine; N¹-(2-fluoroethyl)-N³-(3-((2-(4-methoxyphenyl)quinolin-4-yl)amino)propyl)-N¹,N³-dimethylpropane-1,3-diamine; 3-((3-((2-(4-Methoxyphenyl)quinolin-4-yl)amino)propyl)amino)-propan-I-ol; N-(3-(3-methoxypropylamino)propyl)-2-(4-methoxyphenyl)quinolin-4-amine; N-(3-(2-(4-Methoxyphenyl)quinolin-4-ylamino)propyl)-N-(3-aminopropyl) acetamide; N¹-(Cyclopropylmethyl)-N¹-(3-((2-(4-methoxyphenyl)quinolin-4-yl)amino)propyl)-N³,N³-dimethylpropane-1,3-diamine: tert-Butyl3-(((3-(dimethylamino)propyl) (3-((2-(4-methoxyphenyl)quinolin-4-yl)amino)propyl)amino)methyl) azetidine-1-carboxylate; tert-Butyl4-(((3-(dimethylamino)propyl) (3-((2-(4-methoxyphenyl)quinolin-4-yl)amino)propyl)amino)methyl)piperidine-I-carboxylate: N¹-(Azetidin-3-ylmethyl)-N¹-(3-((2-(4-methoxyphenyl)quinolin-4-yl)amino)propyl)-N³,N³-dimethylpropane-1,3-diamine; N¹-(3-((2-(4-Methoxyphenyl)quinolin-4-yl)amino)propyl)-N³,N³-dimethyl-N¹-(piperidin-4-ylmethyl) propane-1,3-diamine; N¹-(2-(4-Methoxyphenyl)quinolin-4-yl)-N³-((I-methyl-IH-imidazol-5-yl)methyl) propane-1,3-diamine; N-(3-(Hexahydropyrrolo[3,4-c]pyrrol-2 (IH)-yl) propyl)-2-(4-methoxyphenyl)quinolin-4-amine; N¹-((6-Aminopyridin-3-yl)methyl)-N³-(2-(4-methoxyphenyl)-quinolin-4-yl)-N¹-methylpropane-1,3-diamine; I-(Dimethylamino)-3-((3-((2-(4-methoxyphenyl)quinolin-4-yl)-amino)propyl)(methyl)amino) propan-2-ol; 1-(3-((3-((2-(4-Methoxyphenyl)quinolin-4-yl)amino)propyl)-methylamino)propyl) piperidin-4-ol; tert-butyl5-(3-((2-(4-methoxyphenyl)quinolin-4-yl)amino)propyl) hexahydro-pyrrolo[3,4-c]pyrrole-2 (IH)-carboxylate; N¹-((6-(dimethylamino)pyridin-3-yl)methyl)-N³-(2-(4-methoxyphenyl)quinolin-4-yl)-N\hat{ }methylpropane-1,3-diamine; N¹-(3-aminopropyl)-N¹-methyl-N³-(2-(4-(piperazin-1-yl)phenyl)quinolin-4-yl) propane-1,3-diamine; N¹-(2-(4-methoxyphenyl)quinolin-4-yl)-N³-methyl-N³-((I-methylpiperidin-4-yl)methyl) propane-1,3-diamine: N¹-(2-(4-methoxyphenyl)quinolin-4-yl)-N³-methyl-N³-(2-(piperidin-2-yl)ethyl) propane-1,3-diamine; tert-Butyl(2-(3-((2-(4-methoxyphenyl)quinolin-4-yl)amino)propyl) octahydrocyclopenta[c]pyrrol-4-yl) carbamate: N¹-(2-(4-Methoxyphenyl)quinolin-4-yl)-N³-(octahydrocyclopenta[c]pyrrol-4-yl) propane-1,3-diamine; N-(3-(4-Aminohexahydrocyclopenta[c]pyrrol-2 (IH)-yl)propyl)-2-(4-methoxyphenyl)quinolin-4-amine; tert-Butyl2-(3-((2-(4-methoxyphenyl)quinolin-4-yl)amino)propyl) hexahydro-IH-pyrrolo[3,4-c]pyridine-5 (6H)-carboxylate; N-(3-(Hexahydro-IH-pyrrolo[3,4-c]pyridin-2 (3H)-yl)propyl)-2-(4-methoxyphenyl)quinolin-4-amine; tert-Butyl4-((3-((2-(4-methoxyphenyl)quinolin-4-yl)amino)propyl)amino) hexahydrocyclopenta[c]pyrrole-2 (IH)-carboxylate; N¹-((I-(Cyclopropylmethyl) piperidin-4-yl)methyl)-N³-(2-(4-methoxypheny\hat{ }quinolin-d-y\hat{ }-N\hat{ }methylpropane-1,3-diamine; N-(2-(I-(3-Aminopropyl) piperidin-2-yl)ethyl)-2-(4-methoxyphenyl)quinolin-4-amine: N¹-(I-(4-Fluorobenzyl) piperidin-4-yl)-N³-(2-(4-methoxyphenyl)quinolin-4-yl) propane-1,3-diamine: tert-Butyl6-(3-((2-(4-methoxyphenyl)quinolin-4-yl)amino)propyl)-2,6-diazaspiro[3.4]octane-2-carboxylate; N-(3-(2,6-Diazaspiro[3.4]octan-6-yl)propyl)-2-(4-methoxyphenyl)quinolin-4-amine: 2-(4-Methoxyphenyl)-N-(3-(4-methyl-1,4-diazepan-1-yl)propyl)quinolin-4-amine: tert-Butyl2-(2-((3-((2-(4-methoxyphenyl)quinolin-4-yl)amino)propyl)amino)ethyl)piperidine-|-carboxylate; N¹-(2-(4-methoxyphenyl)quinolin-4-yl)-N³-(2-(piperidin-2-yl)ethyl) propane-1,3-diamine; N-(3-(4-(3-Aminopropyl) piperazin-1-yl)propyl)-2-(4-methoxyphenyl)quinolin-4-amine; tert-Butyl4-{[(3-{[2-(4-methoxyphenyl)quinolin-4-yl]amino}propyl)(methyl)amino]methyl}piperidine-1-carboxylate; N-(3-(N-Methyl-N-((piperidin-4-yl)methyl)amino)propyl)-2-(4-methoxyphenyl)quinolin-4-amine; N-(3-((2-(4-Methoxyphenyl)quinolin-4-yl)amino)propyl)-4-(2H-tetrazol-5-yl)benzenesulfonamide; N¹-(4-(2H-Tetrazol-5-yl)phenyl)-N³-(2-(4-methoxyphenyl)quinolin-4-yl) propane-1,3-diamine; (1-(3-((2-(4-Methoxyphenyl)quinolin-4-yl)amino)propyl) piperidin-4-yl) methanol; I-(Diethylamino)-3-((3-((2-(4-methoxyphenyl)quinolin-4-yl)amino)propyl)amino) propan-2-ol; N¹-(2-(4-Methoxyphenyl)quinolin-4-yl)-N³-((I-methyl-1H-pyrazol-5-yl)methyl) propane-1,3-diamine; N¹-(2-(4-methoxyphenyl)quinolin-4-yl)-N³-(2-(pyridin-2-yl)ethyl) propane-1,3-diamine; N¹-(2-(4-Methoxyphenyl)quinolin-4-yl)-N³-(4-(piperidin-I-yl)phenyl) propane-1,3-diamine; N-((I-(3-Aminopropyl) piperidin-3-yl)methyl)-2-(4-methoxyphenyl)quinolin-4-amine; N-(3-(2-(6-Methoxy-IH-indol-3-yl)ethylamino)propyl)-2-(4-methoxyphenyl)quinolin-4-amine: tert-Butyl4-{[({I-[(tert-butoxy) carbonyl]piperidin-4-yl}methyl) (3-{[2-(4-methoxyphenyl)-quinolin-4-yl]amino}propyl)amino]methyl}piperidine-|-carboxylate; N-(3-(Bis((piperidin-4-yl)methyl)amino)propyl)-2-(4-methoxyphenyl)quinolin-4-amine; N¹-(4-Methoxybenzyl)-N³-(2-(4-methoxyphenyl)quinolin-4-yl)-N¹-methylpropane-1,3-diamine; tert-Butyl (3-((4-((2-(4-methoxyphenyl)quinolin-4-yl)amino)cyclohexyl)amino)propyl) carbamate; tert-Butyl4-{[(3-{[2-(4-methoxyphenyl)quinolin-4-yl]amino}propyl)amino]methyl}piperidine-1-carboxylate; N¹-(2-(4-methoxyphenyl)quinolin-4-yl)-N³-(3-(pyridin-4-ylamino)propyl) propane-1,3-diamine; 2-(3-(4-((2-(4-Methoxyphenyl)quinolin-4-yl)amino) piperidin-|-yl)propyl) isoindoline-1,3-dione; 2-(3-(3-(((2-(4-Methoxyphenyl)quinolin-4-yl)amino)methyl) piperidin-1-yl)propyl) isoindoline-1,3-dione; N-(3-((3-((2-(4-Methoxyphenyl)quinolin-4-yl)amino)propyl)amino)propyl) methanesulfonamide; 2-(3-(2-(2-((2-(4-Methoxyphenyl)quinolin-4-yl)amino)ethyl) piperidin-I-yl)propyl) isoindoline-1,3-dione; N-(3-(Hexahydropyrrolo[1,2-a]pyrazin-2 (IH)-yl)propyl)-2-(4-methoxyphenyl)quinolin-4-amine; 2-(4-Methoxyphenyl)-N-(l′-methyl-[1,4′-bipiperidine]-4-yl)quinolin-4-amine; N¹-(2-Benzyloctahydrocyclopenta[c]pyrrol-4-yl)-N³-(2-(4-methoxyphenyl)quinolin-4-yl) propane-1,3-diamine; N¹-((I-(Cyclopropylmethyl) piperidin-4-yl)methyl)-N³-(2-(4-methoxyphenyl) \quinolin-4-yl) propane-1,3-diamine; 1-(3-((2-(4-Methoxyphenyl)quinolin-4-yl)amino)propyl)piperidine-4-carboxamide; 2-(4-Methoxyphenyl)-N-(3-(4-methylpiperazin-1-yl)propyl)quinolin-4-amine; 2-(4-Methoxyphenyl)-N-(2-(4-methylpiperazin-1-yl)ethyl)quinolin-4-amine; 2-(4-Methoxyphenyl)-N-(3-morpholinopropyl)quinolin-4-amine; 2-(7-Amino-4-methyl-2-oxo-2H-chromen-3-yl)-N-(3-((3-((2-(4-methoxyphenyl)quinolin-4-yl)amino)propyl)(methyl)amino)propyl) acetamide; 5-(dimethylamino)-N-{3-[(3-{[2-(4-methoxyphenyl)quinolin-4yl]amino}propyl)(methyl)amino]propyl}naphthalene-I-sulfonamide; cis-N¹-(6,7-dimethoxy-2-(4-(piperazin-1-yl)phenyl)quinolin-4-yl)cyclobutane-1,3-diamine; trans-/V1-(6,7-dimethoxy-2-(4-(piperazin-1-yl)phenyl)quinolin-4-yl)cyclobutane-1,3-diamine; trans-N¹-(6,7-dimethoxy-2-(4-(piperazin-1-yl)phenyl)quinolin-4-yl)cyclohexane-1,4-diamine; N¹-(6,7-dimethoxy-2-(4-(piperazin-1-yl)phenyl)quinolin-4-yl) propane-1,3-diamine; cis-N¹-(2-(4-(piperazin-1-yl)phenyl)quinolin-4-yl)cyclobutane-1,3-diamine; N¹-(2-(4-(5,6-dihydropyrrolo[3,4-c]pyrazol-2 (4H)-yl)phenyl) quinolin-4-yl) propane-1,3-diamine; cis-N¹-(6,7-dimethoxy-2-(4-(piperazin-1-yl)phenyl)quinolin-4-yl)cyclohexane-1,4-diamine; N¹-(2-(4-(piperazin-1-yl)phenyl)quinolin-4-yl) propane-1,3-diamine; N¹-(6,7-dimethoxy-2-(4-(piperazin-1-yl)phenyl)quinolin-4-yl)-N³,N³-dimethylpropane-1,3-diamine; N¹-(3-aminopropyl)-N¹-methyl-N³-(2-(3-(piperazin-1-yl)phenyl)quinolin-4-yl) propane-1,3-diamine; N¹-(6,7-dimethoxy-2-(4-methoxyphenyl)quinolin-4-yl)cyclohexane-1,4-diamine; N¹-(2-(4-(piperazin-1-yl)phenyl)quinolin-4-yl)cyclohexane-1,4-diamine; 2-amino-1-(4-(4-(4-((3-(dimethylamino)propyl)amino)quinolin-2-yl)phenyl) piperazin-1-yl)-3-methylbutan-1-one; 2-amino-1-(4-(4-(4-((3-(dimethylamino)propyl)amino)quinolin-2-yl)phenyl) piperazin-1-yl) propan-1-one; N¹-(2-(I-(1-methylpiperidin-4-yl)-IH-pyrazol-4-yl)quinolin-4-yl) propane-1,3-diamine; N¹-(3-aminopropyl)-N³-(6,7-dimethoxy-2-(4-methoxyphenyl)quinolin-4-yl)-N¹-methylpropane-1,3-diamine; N¹-(2-(4-(piperidin-4-yl)phenyl)quinolin-4-yl) propane-1,3-diamine; N¹-(2-(I-(piperidin-4-yl)-IH-pyrazol-4-yl)quinolin-4-yl) propane-1,3-diamine; trans-N¹-(6,7-dimethoxy-2-(4-methoxyphenyl)quinolin-4-yl)cyclobutane-1,3-diamine;-dimethyl-N³-(2-(3-(piperazin-1-yl)phenyl)quinolin-4-yl) propane-1,3-diamine;-dimethyl-N³-(2-(4-(piperazin-1-yl)phenyl)quinolin-4-yl) propane-1,3-diamine;-(2-(I-(2-(pyrrolidin-1-yl)ethyl)-IH-pyrazol-4-yl)quinolin-4-yl) propane-1,3-diamine; trans-N¹-(6,7-dimethoxy-2-(4-methoxyphenyl)quinolin-4-yl)cyclohexane-1,4-diamine; N-(I-(4-aminocyclohexyl) piperidin-4-yl)-2-(4-methoxyphenyl)quinolin-4-amine;-(6-fluoro-2-(4-(piperazin-1-yl)phenyl)quinolin-4-yl)-N³-dimethylpropane-1,3-diamine; N-(1-(3-aminopropyl) piperidin-4-yl)-2-(4-methoxyphenyl)quinolin-4-amine; cis-N¹-(6,7-dimethoxy-2-(4-methoxyphenyl)quinolin-4-yl)cyclohexane-1,4-diamine; 2-amino-N-(1-((1-(4-(4-(4-((3-(dimethylamino)propyl)amino)quinolin-2-yl)phenyl) piperazin-1-yl)-3-methyl-1-oxobutan-2-yl)amino)-3-methyl-1-oxobutan-2-yl)-3-methylbutanamide;-dimethyl-N³-(2-(1-(1-methylpiperidin-4-yl)-1H-pyrazol-4-yl)quinolin-4-yl) propane-1,3-diamine;-(6,7-dimethoxy-2-(4-methoxyphenyl)quinolin-4-yl)-N³-dimethylpropane-1,3-diamine;-(6,7-dimethoxy-2-(4-methoxyphenyl)quinolin-4-yl) propane-1,3-diamine;-dimethyl-N³-(2-(4-(4-methylpiperazin-1-yl)phenyl)quinolin-4-yl) propane-1,3-diamine; 2-amino-N-(I-(4-(4-(4-((3-(dimethylamino)propyl)amino)quinolin-2-yl)phenyl) piperazin-1-yl)-3-methyl-1-oxobutan-2-yl)-3-methylbutanamide; cis-N¹-(2-(4-methoxyphenyl)quinolin-4-yl)cyclobutane-1,3-diamine;-([2,6′-biquinolin]-4-yl) propane-1,3-diamine; 4-(4-(4-((3-(dimethylamino)propyl)amino)quinolin-2-yl)phenyl)-1-methylpiperazin-2-one; N¹-(2-(piperidin-4-yl)quinolin-4-yl) propane-1,3-diamine; N¹-(2-(2,3-dihydrobenzofiiran-5-yl)quinolin-4-yl) propane-1,3-diamine; N¹-(2-(4-(methoxy-d3)phenyl)quinolin-4-yl) propane-1,3-diamine; N¹-([2,6′-biquinolin]-4-yl)-N³-dimethylpropane-1,3-diamine; N¹-(2-(4-methoxyphenyl)quinolin-4-yl)cyclobutane-1,3-diamine; N¹-(2-(1,2,3,6-tetrahydropyridin-4-yl)quinolin-4-yl) propane-1,3-diamine; N¹-(2-(4-methoxyphenyl)quinolin-4-yl)cyclohexane-1,4-diamine; 2-(4-methoxyphenyl)-N-(piperidin-4-yl)quinolin-4-amine; N¹-(2-(4-(methoxy-d3)phenyl)quinolin-4-yl)-N³-dimethylpropane-1,3-diamine; N¹-(2-(benzofiiran-5-yl)quinolin-4-yl)-N³-dimethylpropane-1,3-diamine; N¹-(2-(I-methyl-1H-pyrazol-4-yl)quinolin-4-yl) propane-1,3-diamine; N¹-(2-(1-(azetidin-3-yl)-1H-pyrazol-4-yl)quinolin-4-yl)-N³,N³-dimethylpropane-1,3-diamine; N¹-(2-(I-cyclopropyl-1H-pyrazol-4-yl)quinolin-4-yl)-N³-dimethylpropane-1,3-diamine; N¹-(2-(cyclohex-1-en-1-yl)quinolin-4-yl) propane-1,3-diamine; (S)-2-amino-N-(3-((2-(4-methoxyphenyl)quinolin-4-yl)amino)propyl)-N-methylpropanamide; N¹-(2-(4-methoxyphenyl)quinolin-4-yl)-N³-bis(methyl-d3) propane-1,3-diamine; N¹-(2-(furan-2-yl)quinolin-4-yl) propane-1,3-diamine; N¹-(5-fluoro-2-(4-methoxyphenyl)quinolin-4-yl)-N³-dimethylpropane-1,3-diamine; N¹-(2-(I-(2-morpholinoethyl)-IH-pyrazol-4-yl)quinolin-4-yl) propane-1,3-diamine; 3-((2-(4-methoxyphenyl)quinolin-4-yl)amino) propan-I-ol; N¹-(2-(2-methoxypyrimidin-5-yl)quinolin-4-yl)-N³,N³-dimethylpropane-1,3-diamine; N³,N³-dimethyl-N³-(2-methylquinolin-4-yl) propane-1,3-diamine; 3-((2-(4-methoxyphenyl)quinolin-4-yl)amino)-N, N-dimethylpropanamide; N¹-(2-(6-methoxypyridin-3-yl)quinolin-4-yl)-N³-dimethylpropane-1,3-diamine; N¹-(2,2-difluoro-6-(4-methoxyphenyl)-[1,3]dioxolo[4,5-g]quinolin-8-yl)-N³-dimethylpropane-1,3-diamine; N-(4,4-difluorocyclohexyl)-2-(4-methoxyphenyl)quinolin-4-amine; tert-butyl3-((2-(4-methoxyphenyl)quinolin-4-yl)amino) propanoate; N¹-(6-methoxy-2-(trifluoromethyl)quinolin-4-yl)-N³-dimethylpropane-1,3-diamine; N¹-(6-methoxy-7-(3-(pyrrolidin-I-yl) propoxy)quinolin-4-yl)-N³,N³-dimethylpropane-1,3-diamine; 2-cyclohexyl-N-(I-isopropylpiperidin-4-yl)-6-methoxy-7-(3-(pyrrolidin-I-yl) propoxy)quinolin-4-amine; N¹-(3-aminopropyl)-N³-(6,7-dimethoxyquinolin-4-y-methylpropane-1,3-diamine; 6-methoxy-N-(I-methylpiperidin-4-yl)-7-(3-(pyrrolidin-1-yl) propoxy)quinolin-4-amine; N¹-(6,7-dimethoxyquinolin-4-yl)-N³,N³-diethylpropane-1,3-diamine; 6,7-dimethoxy-N-(I′-methyl-[1,4′-bipiperidin]-4-yl)quinolin-4-amine; N¹-(3-aminopropyl)-N³. (7-chloroquinolin-4-yl)-N¹-methylpropane-1,3-diamine; N¹-(3-aminopropyl)-N³-(2-(4-(dimethylamino)phenyl)quinolin-4-yl)-N¹-methylpropane-1,3-diamine, an enantiomer, a diastereomer, a hydrate, a solvate, a pharmaceutically acceptable salt, an isotopic analog, a prodrug, or a complex thereof.

### Formula 201

In an embodiment, the TDP-43 modulator may be selected from one of the following compounds according to Formula 201. Such compounds are described in International Publication No. WO2022178338A2, published Aug. 25, 2022, and corresponding to International Application No. PCT/US2022/017116 filed Feb. 20, 2022; which is incorporated by reference in its entirety herein. In the case of any conflict of terminology in the context of Formula 201, these references incorporated by reference herein control.

In an embodiment, the TDP-43 modulator is a compound according to Formula 201:

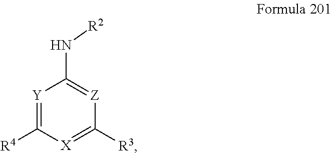

an enantiomer, a diastereomer, a hydrate, a solvate, a pharmaceutically acceptable salt, an isotopic analog, a prodrug or a complex thereof, wherein, X, Y, and Z are each independently N or CH; R² is an amino(C₃-Cv)cycloalkyl group or a (C₁-C₅alkyl)₂amino(C₃-Cv)cycloalkyl group, wherein the (Ci-C alkyl) attached to “amino” are optionally connected to form a ring; R³ is a substituted or unsubstituted phenyl group; and R⁴ is an amino(C₃-Cv)cycloalkyl group, a (C₁-C₅alkyl)₂amino(C₃-Cv)cycloalkyl group wherein the (C i-C alkyl) are optionally connected to form a ring, a heterocyclyl (C₂-C₅)alkyloxy group, or a heteroaryl (C2-C₅)alkyloxy group, wherein “heterocyclyl” is a substituted or unsubstituted nitrogen-containing non-aromatic mono-heterocycle and wherein “heteroaryl” is a substituted or substituted nitrogen-containing aromatic mono-heterocycle.

In further embodiments, X is CH, Y is CH, and Z is CH.

In further embodiments, X is N, Y is CH, and Z is CH.

In further embodiments, X is CH, Y is N, and Z is CH.

In further embodiments, X is CH, Y is CH, and Z is N.

In further embodiments, X is N, Y is N, and Z is CH.

In further embodiments, X is CH, Y is N, and Z is N.

In further embodiments, X is N, Y is CH, and Z is N.

In further embodiments, X is N, Y is N, and Z is N.

In further embodiments, R² is an amino(C3-C7)cycloalkyl group selected from an aminocyclopropyl group, an aminocyclobutyl group, an aminocyclopentyl group, an aminocyclohexyl group, or an aminocycloheptyl group, each of which has either cis-configuration or trans-configuration.

In further embodiments, R² is a (C₁-C₅alkyl)₂amino(C3-C7)cycloalkyl group selected from a (C₁-C₅alkyl)₂aminocyclopropyl group, a (C₁-C₅alkyl)₂aminocyclobutyl group, a (C₁-C₅alkyl)₂aminocyclopentyl group, a (C₁-C₅alkyl)₂aminocyclohexyl group, or a (C₁-C₅alkyl)₂aminocycloheptyl group, each of which has either cis-configuration or trans configuration.

In further embodiments, R³ is a substituted or unsubstituted phenyl group.

In further embodiments, R³ is a phenyl group, a chlorophenyl group, or a methoxyphenyl group.

In further embodiments, R⁴ is a (C₀-C₅alkyl)₂amino(C₂-C₅)alkyloxy group selected from a (C₁-C₅alkyl)₂aminoethyloxy group, a (C₁-C₅alkyl)₂aminopropyloxy group, a (C₁-C₅alkyl)₂aminobutyloxy group, and a (C₁-C₅alkyl)₂aminopentyloxy group, wherein two “(C₁-C₅alkyl)” are optionally connected to form a ring.

In further embodiments, the ring is a substituted or unsubstituted azetidine, a substituted or unsubstituted pyrrolidine, or a substituted or unsubstituted piperidine.

In further embodiments, R⁴ is a (C₀-C₅alkyl)₂amino(C₂-C₅)alkyl group selected from a (C₁-C₅alkyl)₂aminoethyl group, a (C₁-C₅alkyl)₂aminopentyl group, a (C₁-C₅alkyl)₂aminobutyl group, and a (C₁-C₅alkyl)₂aminopentyl group, wherein two “(C₁-C₅alkyl)” are optionally connected to form a ring.

In further embodiments, the ring is a substituted or unsubstituted azetidine, a substituted or unsubstituted pyrrolidine, or a substituted or unsubstituted piperidine.

In further embodiments, R⁴ is a heterocyclyl (C₂-C₅)alkyloxy group or a heteroaryl (C₂-C₅)alkyloxy group, wherein “heterocyclyl” is a substituted or unsubstituted nitrogen-containing non-aromatic mono-heterocycle and wherein “heteroaryl” is a substituted or substituted nitrogen-containing aromatic mono-heterocycle.

In further embodiments, “heterocyclyl” and the “heteroaryl” are connected to the “(C₂-C₅)alkyloxy” through a nitrogen atom.

In further embodiments, R⁴ is a substituted or unsubstituted pyrrolidinoethyloxy group or substituted or unsubstituted pyrrolidinopropyloxy group.

In further embodiments, the compound is according to formula 201, an enantiomer, a diastereomer, a hydrate, a solvate, a pharmaceutically acceptable salt, an isotopic analog, a prodrug or a complex thereof, wherein, X, Y, and Z are each independently N or CH; R² is a substituted or unsubstituted C₁-C₂₀ linear, branched, or cyclic organic group including at least one nitrogen; and R³ and R⁴ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

In further embodiments,

### Formula 202

In an embodiment, the TDP-43 modulator may be selected from one of the following compounds according to Formula 202. Such compounds are described in International Publication No. WO2022178338A2, published Aug. 25, 2022 and corresponding to International Application No. PCT/US2022/017116 filed Feb. 20, 2022; which is incorporated by reference in its entirety herein. In the case of any conflict of terminology in the context of Formula 202, these references incorporated by reference herein control.

In an embodiment, the TDP-43 modulator is a compound according to Formula 202:

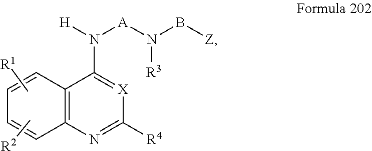

an enantiomer, a diastereomer, a hydrate, a solvate, a pharmaceutically acceptable salt, an isotopic analog, a prodrug or a complex thereof, wherein, X is N or CH; R¹ and R² are the same or different and are each independently H, halogen, C₁-C₄ alkyl, C₁-C₄ alkoxy, CF₃, or CF₃O, wherein C₁-C₄ alkyl and C₁-C₄ alkoxy are optionally substituted with at least one fluorine; R³ is H, C₁-C7 alkyl, C4-C7 carbocyclic, aryl, heteroaryl, heterocyclic, heterocyclic alkyl, wherein R³ is optionally taken together with either A or B to form a ring consisting of 4-7 ring members; R⁴=phenyl or phenyl substituted with C₁-C₄ alkyl, C₁-C₄ alkoxy, CF₃, CF₃O, halogen, amino, or sulfonamide; A=(CH₂)ₙ, wherein n is 2-4, wherein A and R³ are optionally taken together to form a ring consisting of 4-7 ring members; B═(CH₂)ₙ, wherein n is 2-6, wherein B and R³ are optionally taken together to form a ring consisting of 4-7 members, and wherein B is optionally substituted with one or more substituents selected from C₁-C₄ alkyl, hydroxy, C₁-C₄ alkoxy, CF₃, and CF₃O; and Z is OH, C₁-C₄ alkoxy, OCF₃, or N with one or more of H, C₁-C₄ alkyl, heteroaryl, substituted heteroaryl, C₁-C₄ sulfonamido, substituted amido.

In further embodiments, X═CH; R¹ and R² are each H, R³ is H or Me, R⁴ is 4-methoxyphenyl, A is (CH₂) 3, B is (CH₂) n, wherein n is 2-4 and Z═N(R⁶)₂, wherein each R⁶ is independently H, C₁-C₄ alkyl, heteroaryl, substituted heteroaryl, C₁-C₄ sulfonamido, or substituted amido.

### Formula 203-204

In an embodiment, the TDP-43 modulator may be selected from one of the following compounds according to any one of Formula 203-204. Such compounds are described in International Publication No. WO2022178338A2, published Aug. 25, 2022, and corresponding to International Application No. PCT/US2022/017116 filed Feb. 20, 2022; which is incorporated by reference in its entirety herein. In the case of any conflict of terminology in the context of Formula 203-204, these references incorporated by reference herein control.

In an embodiment, the TDP-43 modulator is a compound according to any one of Formula 203-204:

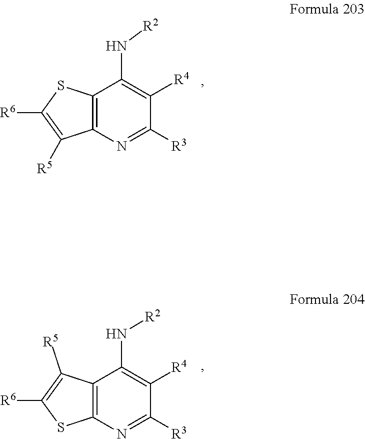

an enantiomer, a diastereomer, a hydrate, a solvate, a pharmaceutically acceptable salt, an isotopic analog, a prodrug or a complex thereof, wherein, R² is a substituted or unsubstituted C₁-C₂₀ linear, branched, or cyclic organic group including at least one nitrogen; and R³, R⁴, R⁵, and R⁶ are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

### Formula 205

In an embodiment, the TDP-43 modulator may be selected from one of the following compounds according to Formula 205. Such compounds are described in International Publication No. WO2022178338A2, published Aug. 25, 2022, and corresponding to International Application No. PCT/US2022/017116 filed Feb. 20, 2022; which is incorporated by reference in its entirety herein. In the case of any conflict of terminology in the context of Formula 205, these references incorporated by reference herein control.

In an embodiment, the TDP-43 modulator is a compound according to Formula 205:

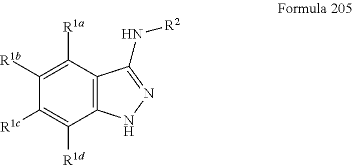

an enantiomer, a diastereomer, a hydrate, a solvate, a pharmaceutically acceptable salt, an isotopic analog, a prodrug or a complex thereof, wherein, R² is a substituted or unsubstituted C₁-C₂₀ linear, branched, or cyclic organic group including at least one nitrogen; and R^{1a}, R^{1b}, R^{1c}, and R^{1d} are each independently hydrogen, halogen, a hydroxyl group, a cyano group, a nitro group, a substituted or unsubstituted C₀-C₁₀ amino group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

### Formula 206

In an embodiment, the TDP-43 modulator may be selected from one of the following compounds according to Formula 206. Such compounds are described in International Publication No. WO2022178338A2, published Aug. 25, 2022, and corresponding to International Application No. PCT/US2022/017116 filed Feb. 20, 2022; which is incorporated by reference in its entirety herein. In the case of any conflict of terminology in the context of Formula 206, these references incorporated by reference herein control.

In an embodiment, the TDP-43 modulator is a compound according to Formula 206:

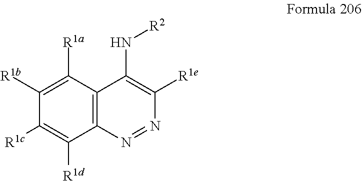

an enantiomer, a diastereomer, a hydrate, a solvate, a pharmaceutically acceptable salt, an isotopic analog, a prodrug or a complex thereof, wherein, R² is a substituted or unsubstituted C₁-C₂₀ linear, branched, or cyclic organic group including at least one nitrogen; and R^{1a}, R^{1b}, 1^{1c}, R^{1d}, and R^{1e} are each independently hydrogen, halogen, a hydroxyl group, a cyano group, a nitro group, a substituted or unsubstituted Co-Cio amino group, a substituted or unsubstituted Ci-Cio alkyl group, a substituted or unsubstituted C₂-C₁₀ alkenyl group, a substituted or unsubstituted C₂-C₁₀ alkynyl group, and a substituted or unsubstituted C₁-C₁₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₂₀ aryl group, a substituted or unsubstituted C₆-C₂₀ aryloxy group, a substituted or unsubstituted C₆-C₂₀ arylthio group, a substituted or unsubstituted C₂-C₂₀ heteroaryl group, a substituted or unsubstituted C₂-C₂₀ heteroaryloxy group, or a substituted or unsubstituted C₂-C₂₀ heteroarylthio group.

### Further TDP-43 Modulator Embodiments

The TDP-43 modulator may be present as isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure at levels higher than natural abundance include isotopes of hydrogen, carbon, nitrogen, oxygen, phosphorous, fluorine and chlorine, such as, but not limited to ²H (deuterium, D), ³H (tritium), ⁿC, ¹³C, ¹⁴C, ¹⁵N, ¹⁸F, ³¹P, ³²P, ³⁵S, Cl and I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g., humans). Also provided for isotopically labeled compounds described herein are any pharmaceutically acceptable salts, or hydrates, as the case may be.

In some variations, the compounds disclosed herein may be varied such that from 1 to “n” hydrogens attached to a carbon atom is/are replaced by deuterium, in which “n” is the number of hydrogens in the molecule. Such compounds may exhibit increased resistance to metabolism and are thus useful for increasing the half-life of the compound when administered to a subject. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved drug metabolism and pharmacokinetics (DMPK) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures known to those skilled in the art by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as ‘H” or “hydrogen”, the position is understood to have hydrogen at its natural abundance isotopic composition.

### Pharmaceutical Compositions

In an embodiment, provided is a pharmaceutical composition comprising a metal channel activator and a TDP-43 modulator.

In another embodiment, the metal channel activator in said pharmaceutical composition is a potassium channel activator.

In another embodiment, the potassium channel activator in said pharmaceutical composition is a Kv7 channel activator.

In another embodiment, the Kv7 channel activator in said pharmaceutical composition is selected from the group consisting of a Kv7.1 channel activator, a Kv7.2 channel activator, a Kv7.3 channel activator, a Kv7.4 channel activator, a Kv7.5 channel activator, or any combination thereof.

In another embodiment, the Kv7 channel activator in said pharmaceutical composition is a Kv7.2/7.3 channel activator.

In another embodiment, the metal channel activator in said pharmaceutical composition is selected from one or more of the disclosed compounds according to any one or more of formulas 1 to 8.

In another embodiment, the TDP-43 modulator in said pharmaceutical composition is selected from one or more of the disclosed compounds according to any one or more of formulas 200 to 206.

In another embodiment, the TDP-43 modulator in said pharmaceutical composition is selected from one or more of the disclosed compounds according to any one or more of formulas 200 to 206, and the metal channel activator in said pharmaceutical composition is selected from one or more of the disclosed compounds according to any one or more of formulas 1 to 8.

In another embodiment, the TDP-43 modulator or a pharmaceutically acceptable salt thereof in said pharmaceutical composition is in a form of a prodrug.

### Methods of Treating Diseases

The combination therapies disclosed herein are useful for treating or preventing various neurological and neurodegenerative disorders. In an embodiment, disclosed is a method for treating or preventing a disease that involves TDP-43, including administering to a subject any of the above pharmaceutical compositions. The disease may be selected from the group consisting of amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), Alzheimer's disease (AD) related disorders, or any combination thereof.

In another embodiment, disclosed is a method for treating or preventing a disease associated with TDP-43 proteinopathies, including administering to a subject any of the above pharmaceutical compositions.

In another embodiment, disclosed is a method for treating or preventing a disease associated with TDP-43 proteinopathies and/or that can benefit from Kv7 channel activation, including administering to a subject any of the above pharmaceutical compositions.

In another embodiment, disclosed is a method for treating or preventing a disease that can benefit from Kv7 channel activation, including administering to a subject any of the above pharmaceutical compositions.

In another embodiment, disclosed is a method for treating or preventing diseases that involve excess amounts of TDP-43 in the cytosol, including administering to a subject any of the above pharmaceutical compositions.

In another embodiment, disclosed is a method of use of the TDP-43 modulators of the present invention as positron emission tomography (PET) imaging agents, including administering to a subject any of the pharmaceutical compositions.

In another embodiment, disclosed is a method of use of the TDP-43 modulators of the present invention as single-photon emission computed tomography (SPECT) imaging agents, wherein the method includes administering to a subject any of the above pharmaceutical compositions including an isotopically labeled metal channel activator, an isotopically labeled TDP-43 modulator, or any combination thereof. The pharmaceutical composition may be administered in a combination with at least one other agent known to treat amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), Alzheimer's disease (AD) related disorders, or any combination thereof.

In another embodiment, the combination of Kv7 opener and TDP-43 modulator further includes at least one other agent. In some embodiments, the at least one other agent is riluzole, troriluzole, and/or edavarone.

In further embodiments, a kit is provided for treating a patient afflicted with a disorder involving metal channel dysfunction and TDP-43, said kit comprising:
(a) the metal channel activator; and
(b) instructions for administering said metal channel activator in combination with

In further embodiments, a kit is provided for treating a patient wherein the patient is afflicted with a disorder involving metal channel dysfunction and TDP-43, said kit comprising:
(a) the metal channel activator; and
(b) instructions for administering said metal channel activator in combination with a TDP-43 modulator by one of the aforementioned methods.

In further embodiments, a kit is provided for treating a patient wherein the patient is afflicted with a disorder involving metal channel dysfunction and TDP-43, said kit comprising:
(a) the TDP-43 modulator; and
(b) instructions for administering said TDP-43 modulator with a metal channel activator by one of the aforementioned methods.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

### Pharmaceutical Compositions and Methods of Treatment

Compounds are generally given as pharmaceutical compositions comprised of a therapeutically effective amount of one or more of a compound of any one of Formulas 1-8, Formulas 200-206, or pharmaceutically acceptable salt(s) thereof, and a pharmaceutically acceptable carrier, further optionally containing conventional excipients. A therapeutically effective amount is the amount needed to provide a meaningful patient benefit as determined by practitioners in that art. Pharmaceutically acceptable carriers are those having acceptable safety profiles which are conventionally known. Compositions encompass common solid and liquid forms including but not limited to capsules, tablets, lozenges, liquid suspensions, syrups, elixirs, and solutions. Solid compositions may by formulated to timed or sustained release. Compositions are made using common formulation techniques, such as the aforementioned solid and liquid forms, conventional excipients, such as binding and wetting agents, and vehicles, such as water and alcohols.

The TDP-43 modulators of the present invention may be given orally, sublingually, intranasally, buccally, subcutaneously or in any other suitable means of delivery in table 1. The TDP-43 modulators may be in the form of a prodrug, which releases the agent in the body, a sustained release vehicle, a delayed release vehicle, or any other suitable delivery form. The TDP-43 modulator and the metal channel activator may be delivered simultaneously or sequentially. If the agents are delivered sequentially, either agent may be dosed first, and the separation of time may include finishing the dosing of one agent completely before commencing the dosage of the other or they may be intermingled in time. Typically, the TDP-43 modulator is administered at a time proximate to the administration of the metal channel activator, e.g., within 1 week, 1 day, 1 hour, 1 minute before or after the metal channel activator or simultaneously with the metal channel activator.

The metal channel activators of the present invention may be given orally, sublingually, intranasally, buccally, subcutaneously or in any other suitable means of delivery in table 1. Metal channel activators may be given in the form of a prodrug, which will release the active compound in the body, a delayed release formulation, which will release the active compound after a time delay, a sustained release formulation, which will release the active compound slowly over time, or any other suitable formulation to deliver the active ingredient. The metal channel activator may be delivered simultaneously or sequentially with the TDP-43 modulator. If the agents are delivered sequentially, either agent may be dosed first, and the separation of time may include finishing the dosing of one agent completely before commencing the dosage of the other or they may be intermingled in time. Typically, the metal channel activator will be administered at a time nearing the administration of the TDP-43 modulator e.g., within 1 week, 1 day, 1 hour, 1 minute before or after the TDP-43 modulator or simultaneously with the metal channel activator.

The dose of the TDP-43 modulator and metal channel activator for use together may depend on the subject to be treated inclusive of the age, sex, weight, and general health condition thereof. In this regard, precise amounts of the agent(s) for administration will depend on the judgment of the practitioner. In determining the effective amount of the TDP-43 modulator and metal channel activator to be administered in the treatment or reducing of the conditions associated with the symptoms and disorders, the physician may evaluate clinical factors including symptoms severity or progression of the disorder. In some conditions, a rapid absorption of the TDP-43 modulator or metal channel activator may be desirable. The effective amount of the treatment will vary depending on the subject and disease state being treated, the severity of the affliction and the manner of administration, and may be determined routinely by one of ordinary skill in the art.

Some of the TDP-43 modulators and metal channel activators can be administered sublingually. The sublingual formulation may be administered in an effective amount to a subject in need thereof. The subject may be an animal or human. When the TDP-43 modulator or metal channel activator is prepared as a sublingual formulation, the sublingually administered chemical agent or the drug can diffuse into capillaries through mucous membrane under the tongue, and then enter venous circulation of the subject. As such, sublingual administration may have advantages over oral administration as allowing for direct or faster entry to venous circulation, without risks of degradation in gastrointestinal tract, alteration by drug metabolism in liver and the like.

A sublingual formulation useful in the present invention comprises an effective amount of a TDP-43 modulator, metal channel activator, or pharmaceutically acceptable salts, solvates, anomers, enantiomers, hydrates, or prodrugs thereof. The formulation provides sufficient solubility for a TDP-43 modulator and/or metal channel activator to be incorporated into the sublingual formulation at relatively large doses and sublingually delivered. The formulation is preferably a modified oral disintegrating formulation of a TDP-43 modulator and/or metal channel activator. The excipients, including mannitol and gelatin, are blended, solubilized with water and deaerated before being mixed with the active pharmaceutical ingredient (or “API”), a TDP-43 modulator and/or a metal channel activator, which have been milled separately. Particle size of the API (D50) is less than about 2 microns. The mixture is lyophilized by flash freezing and then freeze-dried. The formulation has good oral palatability. The effective amount of a TDP-43 modulator and/or metal channel activator for the sublingual formulation useful in the present invention to achieve a lower therapeutic dose may be less than that of orally administered agent. Moreover, effective dose of the sublingual formulation of the TDP-43 modulator and/or metal channel activator may be about 1 to 95% of that of the orally administered agent. Sublingual formulations of the metal channel activator and/or TDP-43 modulator may also have improved properties.

In one aspect of the invention, the TDP-43 modulator and/or metal channel activator is provided in a sublingual formulation in a form of an orally dissolving or disintegrating tablet (ODT). The ODT as used herein may be prepared by mixing the TDP-43 modulator and/or the metal channel activator with water-soluble diluents and compressed in a tablet. A suspension comprising the active product may be prepared with appropriate excipients and the suspension may be dispensed into blister packs and freeze-dried. An exemplary freeze-dried preparation platform that could be used for the ODT is the ZYDIS® (Catalent, Somerset, NJ, USA) formulation. In particular, the excipients, including water, are blended and the TDP-43 modulator and/or metal channel activator are separately milled to size and mixed with the excipients. The suspension then undergoes lyophilization by flash freezing and freeze drying. Other methods of preparing ODTs may be used without limitation, and detailed description of general methods thereof have been disclosed, for example, in U.S. Pat. Nos. 5,631,023; 5,837,287; 6,149,938; 6,212,791; 6,284,270; 6,316,029; 6,465,010; 6,471,992; 6,471,992; 6,509,040; 6,814,978; 6,908,626; 6,908,626; 6,982,251; 7,282,217; 7,425,341; 7,939,105; 7,993,674; 8,048,449; 8,127,516; 8,158,152; 8,221,480; 8,256,233; and 8,313,768, each of which is incorporated herein by reference in its entirety.

The clinical or therapeutic effect of the sublingually formulated TDP-43 modulator may have an improved pharmacokinetic profile for the pharmaceutical agent as measured by standard testing parameters. When the TDP-43 modulator is administered sublingually, the Tₘₐₓ, Cₘₐₓ and AUC of the drug may be improved compared to the same dose of the orally administered version of the same compound. For example, the sublingual formulation of the TDP-43 modulator may have a greater Cₘₐₓ than the orally administered TDP-43 modulator to provide a therapeutically beneficial effect. The sublingual formulation of the TDP-43 modulator may have an earlier or lesser Tₘₐₓ than the orally administered TDP-43 modulator to provide a therapeutically beneficial effect and in some instances, a more rapid therapeutic effect. Alternatively, the sublingual formulation of the TDP-43 modulator may have a greater AUC per milligram of the agent than the orally administered TDP-43 modulator. In addition, as the TDP-43 modulator may make the metal channel activator more effective, lesser amounts of the metal channel activator may be needed to achieve the same results lessening of the inherent side effects.

The clinical or therapeutic effect of the sublingually formulated metal channel activator may have an improved pharmacokinetic profile for the pharmaceutical agent as measured by standard testing parameters. When the metal channel activator is administered sublingually, the Tₘₐₓ, Cₘₐₓ and AUC of the drug may be improved compared to the same dose of the orally administered version of the same compound. For example, the sublingual formulation of the metal channel activator may have a greater Cₘₐₓ than the orally administered metal channel activator to provide a therapeutically beneficial effect. The sublingual formulation of the metal modulator may have an earlier or lesser Tₘₐₓ than the orally administered metal channel activator to provide a therapeutically beneficial effect and in some instances, a more rapid therapeutic effect. Alternatively, the sublingual formulation of the metal channel activator may have a greater AUC per milligram of the agent than the orally administered metal channel activator. In addition, as the metal channel activator may make the TDP-43 modulator more effective, lesser amounts of the TDP-43 modulator may be needed to achieve the same results lessening of the inherent side effects.

The disclosure and methods encompass all conventional modes of administration including oral, parenteral, intranasal, sublingual, topical, and transdermal methods. Among other routes of administration, the standard routes of administration described by the FDA are contemplated herein as shown in Table 1 below (FDA Routes of Administration; retrieved from www.fda.gov; content current as of Nov. 1, 2022).

TABLE 1
FDA Routes of Administration

| | | | | NCI |
|---|---|---|---|---|
| | | SHORT | FDA | CONCEPT |
| NAME | DEFINITION | NAME | CODE | ID |
| AURICULAR (OTIC) | Administration to or by way of | OTIC | 013 | C38192 |
| | the ear. | | | |
| BUCCAL | Administration directed | BUCCAL | 030 | C38193 |
| | toward the cheek, generally | | | |
| | from within the mouth. | | | |
| CONJUNCTIVAL | Administration to the | CONJUNC | 068 | C38194 |
| | conjunctiva, the delicate | | | |
| | membrane that lines the | | | |
| | eyelids and covers the | | | |
| | exposed surface of the | | | |
| | eyeball. | | | |
| CUTANEOUS | Administration to the skin. | CUTAN | 130 | C38675 |
| DENTAL | Administration to a tooth or | DENTAL | 038 | C38197 |
| | teeth. | | | |
| ELECTRO-OSMOSIS | Administration of through the | EL-OSMOS | 357 | C38633 |
| | diffusion of substance | | | |
| | through a membrane in an | | | |
| | electric field. | | | |
| ENDOCERVICAL | Administration within the | E-CERVIC | 131 | C38205 |
| | canal of the cervix | | | |
| | uteri. Synonymous with the | | | |
| | term intracervical. | | | |
| ENDOSINUSIAL | Administration within the | E-SINUS | 133 | C38206 |
| | nasal sinuses of the head. | | | |
| ENDOTRACHEAL | Administration directly into | E-TRACHE | 401 | C38208 |
| | the trachea. | | | |
| ENTERAL | Administration directly into | ENTER | 313 | C38209 |
| | the intestines. | | | |
| EPIDURAL | Administration upon or over | EPIDUR | 009 | C38210 |
| | the dura mater. | | | |
| EXTRA-AMNIOTIC | Administration to the outside | X-AMNI | 402 | C38211 |
| | of the membrane enveloping | | | |
| | the fetus | | | |
| EXTRACORPOREAL | Administration outside of the | X-CORPOR | 057 | C38212 |
| | body. | | | |
| HEMODIALYSIS | Administration through | HEMO | 140 | C38200 |
| | hemodialysate fluid. | | | |
| INFILTRATION | Administration that results in | INFIL | 361 | C38215 |
| | substances passing into | | | |
| | tissue spaces or into cells. | | | |
| INTERSTITIAL | Administration to or in the | INTERSTIT | 088 | C38219 |
| | interstices of a tissue. | | | |
| INTRA-ABDOMINAL | Administration within the | I-ABDOM | 056 | C38220 |
| | abdomen. | | | |
| INTRA-AMNIOTIC | Administration within the | I-AMNI | 060 | C38221 |
| | amnion. | | | |
| INTRA-ARTERIAL | Administration within an | I-ARTER | 037 | C38222 |
| | artery or arteries. | | | |
| INTRA-ARTICULAR | Administration within a joint. | I-ARTIC | 007 | C38223 |
| INTRABILIARY | Administration within the bile, | I-BILI | 362 | C38224 |
| | bile ducts or gallbladder. | | | |
| INTRABRONCHIAL | Administration within a | I-BRONCHI | 067 | C38225 |
| | bronchus. | | | |
| INTRABURSAL | Administration within a bursa. | I-BURSAL | 025 | C38226 |
| INTRACARDIAC | Administration with the heart. | I-CARDI | 027 | C38227 |
| INTRACARTILAGINOUS | Administration within a | I-CARTIL | 363 | C38228 |
| | cartilage; endochondral. | | | |
| INTRACAUDAL | Administration within the | I-CAUDAL | 413 | C38229 |
| | cauda equina. | | | |
| INTRACAVERNOUS | Administration within a | I-CAVERN | 132 | C38230 |
| | pathologic cavity, such as | | | |
| | occurs in the lung in | | | |
| | tuberculosis. | | | |
| INTRACAVITARY | Administration within a non- | I-CAVIT | 023 | C38231 |
| | pathologic cavity, such as | | | |
| | that of the cervix, uterus, or | | | |
| | penis, or such as that which | | | |
| | is formed as the result of a | | | |
| | wound. | | | |
| INTRACEREBRAL | Administration within the | I-CERE | 404 | C38232 |
| | cerebrum. | | | |
| INTRACISTERNAL | Administration within the | I-CISTERN | 405 | C38233 |
| | cisterna magna | | | |
| | cerebellomedularis. | | | |
| INTRACORNEAL | Administration within the | I-CORNE | 406 | C38234 |
| | cornea (the transparent | | | |
| | structure forming the anterior | | | |
| | part of the fibrous tunic of the | | | |
| | eye). | | | |
| INTRACORONAL, | Administration of a drug | I-CORONAL | 117 | C38217 |
| DENTAL | within a portion of a tooth | | | |
| | which is covered by enamel | | | |
| | and which is separated from | | | |
| | the roots by a slightly | | | |
| | constricted region known as | | | |
| | the neck. | | | |
| INTRACORONARY | Administration within the | I- | 119 | C38218 |
| | coronary arteries. | CORONARY | | |
| INTRACORPORUS | Administration within the | I-CORPOR | 403 | C38235 |
| CAVERNOSUM | dilatable spaces of the | | | |
| | corpora cavernosa of the | | | |
| | penis. | | | |
| INTRADERMAL | Administration within the | I-DERMAL | 008 | C38238 |
| | dermis. | | | |
| INTRADISCAL | Administration within a disc. | I-DISCAL | 121 | C38239 |
| INTRADUCTAL | Administration within the duct | I-DUCTAL | 123 | C38240 |
| | of a gland. | | | |
| INTRADUODENAL | Administration within the | I-DUOD | 047 | C38241 |
| | duodenum. | | | |
| INTRADURAL | Administration within or | I-DURAL | 052 | C38242 |
| | beneath the dura. | | | |
| INTRAEPIDERMAL | Administration within the | I-EPIDERM | 127 | C38243 |
| | epidermis. | | | |
| INTRAESOPHAGEAL | Administration within the | I-ESO | 072 | C38245 |
| | esophagus. | | | |
| INTRAGASTRIC | Administration within the | I-GASTRIC | 046 | C38246 |
| | stomach. | | | |
| INTRAGINGIVAL | Administration within the | I-GINGIV | 307 | C38247 |
| | gingivae. | | | |
| INTRAILEAL | Administration within the | I-ILE | 365 | C38249 |
| | distal portion of the small | | | |
| | intestine, from the jejunum to | | | |
| | the cecum. | | | |
| INTRALESIONAL | Administration within or | I-LESION | 042 | C38250 |
| | introduced directly into a | | | |
| | localized lesion. | | | |
| INTRALUMINAL | Administration within the | I-LUMIN | 310 | C38251 |
| | lumen of a tube. | | | |
| INTRALYMPHATIC | Administration within the | I-LYMPHAT | 352 | C38252 |
| | lymph. | | | |
| INTRAMEDULLARY | Administration within the | I-MEDUL | 408 | C38253 |
| | marrow cavity of a bone. | | | |
| INTRAMENINGEAL | Administration within the | I-MENIN | 409 | C38254 |
| | meninges (the three | | | |
| | membranes that envelope | | | |
| | the brain and spinal cord). | | | |
| INTRAMUSCULAR | Administration within a | IM | 005 | C28161 |
| | muscle. | | | |
| INTRAOCULAR | Administration within the eye. | I-OCUL | 036 | C38255 |
| INTRAOVARIAN | Administration within the | I-OVAR | 354 | C38256 |
| | ovary. | | | |
| INTRAPERICARDIAL | Administration within the | I-PERICARD | 314 | C38257 |
| | pericardium. | | | |
| INTRAPERITONEAL | Administration within the | I-PERITON | 004 | C38258 |
| | peritoneal cavity. | | | |
| INTRAPLEURAL | Administration within the | I-PLEURAL | 043 | C38259 |
| | pleura. | | | |
| INTRAPROSTATIC | Administration within the | I-PROSTAT | 061 | C38260 |
| | prostate gland. | | | |
| INTRAPULMONARY | Administration within the | I-PULMON | 414 | C38261 |
| | lungs or its bronchi. | | | |
| INTRASINAL | Administration within the | I-SINAL | 010 | C38262 |
| | nasal or periorbital sinuses. | | | |
| INTRASPINAL | Administration within the | I-SPINAL | 022 | C38263 |
| | vertebral column. | | | |
| INTRASYNOVIAL | Administration within the | I-SYNOV | 019 | C38264 |
| | synovial cavity of a joint. | | | |
| INTRATENDINOUS | Administration within a | I-TENDIN | 049 | C38265 |
| | tendon. | | | |
| INTRATESTICULAR | Administration within the | I-TESTIC | 110 | C38266 |
| | testicle. | | | |
| INTRATHECAL | Administration within the | IT | 103 | C38267 |
| | cerebrospinal fluid at any | | | |
| | level of the cerebrospinal | | | |
| | axis, including injection into | | | |
| | the cerebral ventricles. | | | |
| INTRATHORACIC | Administration within the | I-THORAC | 006 | C38207 |
| | thorax (internal to the ribs); | | | |
| | synonymous with the term | | | |
| | endothoracic. | | | |
| INTRATUBULAR | Administration within the | I-TUBUL | 353 | C38268 |
| | tubules of an organ. | | | |
| INTRATUMOR | Administration within a tumor. | I-TUMOR | 020 | C38269 |
| INTRATYMPANIC | Administration within the | I-TYMPAN | 366 | C38270 |
| | Auris media. | | | |
| INTRAUTERINE | Administration within the | I-UTER | 028 | C38272 |
| | uterus. | | | |
| INTRAVASCULAR | Administration within a vessel | I-VASC | 021 | C38273 |
| | or vessels. | | | |
| INTRAVENOUS | Administration within or into a | IV | 002 | C38276 |
| | vein or veins. | | | |
| INTRAVENOUS BOLUS | Administration within or into a | IV BOLUS | 138 | C38274 |
| | vein or veins all at once. | | | |
| INTRAVENOUS DRIP | Administration within or into a | IV DRIP | 137 | C38279 |
| | vein or veins over a | | | |
| | sustained period of time. | | | |
| INTRAVENTRICULAR | Administration within a | I-VENTRIC | 048 | C38277 |
| | ventricle. | | | |
| INTRAVESICAL | Administration within the | I-VESIC | 128 | C38278 |
| | bladder. | | | |
| INTRAVITREAL | Administration within the | I-VITRE | 311 | C38280 |
| | vitreous body of the eye. | | | |
| IONTOPHORESIS | Administration by means of | ION | 055 | C38203 |
| | an electric current where ions | | | |
| | of soluble salts migrate into | | | |
| | the tissues of the body. | | | |
| IRRIGATION | Administration to bathe or | IRRIG | 032 | C38281 |
| | flush open wounds or body | | | |
| | cavities. | | | |
| LARYNGEAL | Administration directly upon | LARYN | 364 | C38282 |
| | the larynx. | | | |
| NASAL | Administration to the nose; | NASAL | 014 | C38284 |
| | administered by way of the | | | |
| | nose. | | | |
| NASOGASTRIC | Administration through the | NG | 071 | C38285 |
| | nose and into the stomach, | | | |
| | usually by means of a tube. | | | |
| NOT APPLICABLE | Routes of administration are | NA | 312 | C48623 |
| | not applicable. | | | |
| OCCLUSIVE | Administration by the topical | OCCLUS | 134 | C38286 |
| DRESSING | route which is then covered | | | |
| TECHNIQUE | by a dressing which occludes | | | |
| | the area. | | | |
| OPHTHALMIC | Administration to the external | OPHTHALM | 012 | C38287 |
| | eye. | | | |
| ORAL | Administration to or by way of | ORAL | 001 | C38288 |
| | the mouth. | | | |
| OROPHARYNGEAL | Administration directly to the | ORO | 410 | C38289 |
| | mouth and pharynx. | | | |
| OTHER | Administration is different | OTHER | 135 | C38290 |
| | from others on this list. | | | |
| PARENTERAL | Administration by injection, | PAREN | 411 | C38291 |
| | infusion, or implantation. | | | |
| PERCUTANEOUS | Administration through the | PERCUT | 113 | C38676 |
| | skin. | | | |
| PERIARTICULAR | Administration around a joint. | P-ARTIC | 045 | C38292 |
| PERIDURAL | Administration to the outside | P-DURAL | 050 | C38677 |
| | of the | | | |
| | dura mater of the | | | |
| | spinal cord. | | | |
| PERINEURAL | Administration surrounding a | P-NEURAL | 412 | C38293 |
| | nerve or nerves. | | | |
| PERIODONTAL | Administration around a | P-ODONT | 040 | C38294 |
| | tooth. | | | |
| RECTAL | Administration to the rectum. | RECTAL | 016 | C38295 |
| RESPIRATORY | Administration within the | RESPIR | 136 | C38216 |
| (INHALATION) | respiratory tract by inhaling | | | |
| | orally or nasally for local or | | | |
| | systemic effect. | | | |
| RETROBULBAR | Administration behind the | RETRO | 034 | C38296 |
| | pons or behind the eyeball. | | | |
| SOFT TISSUE | Administration into any soft | SOFT TIS | 109 | C38198 |
| | tissue. | | | |
| SUBARACHNOID | Administration beneath the | S-ARACH | 066 | C38297 |
| | arachnoid. | | | |
| SUBCONJUNCTIVAL | Administration beneath the | S- | 096 | C38298 |
| | conjunctiva. | CONJUNC | | |
| SUBCUTANEOUS | Administration beneath the | SC | 003 | C38299 |
| | skin; | | | |
| | hypodermic. Synonymous | | | |
| | with the term SUBDERMAL. | | | |
| SUBLINGUAL | Administration beneath the | SL | 024 | C38300 |
| | tongue. | | | |
| SUBMUCOSAL | Administration beneath the | S-MUCOS | 053 | C38301 |
| | mucous membrane. | | | |
| TOPICAL | Administration to a particular | TOPIC | 011 | C38304 |
| | spot on the outer surface of | | | |
| | the body. The E2B term | | | |
| | TRANSMAMMARY is a | | | |
| | subset of the term TOPICAL. | | | |
| TRANSDERMAL | Administration through the | T-DERMAL | 358 | C38305 |
| | dermal layer of the skin to the | | | |
| | systemic circulation by | | | |
| | diffusion. | | | |
| TRANSMUCOSAL | Administration across the | T-MUCOS | 122 | C38283 |
| | mucosa. | | | |
| TRANSPLACENTAL | Administration through or | T-PLACENT | 415 | C38307 |
| | across the placenta. | | | |
| TRANSTRACHEAL | Administration through the | T-TRACHE | 355 | C38308 |
| | wall of the trachea. | | | |
| TRANSTYMPANIC | Administration across or | T-TYMPAN | 124 | C38309 |
| | through the tympanic cavity. | | | |
| UNASSIGNED | Route of administration has | UNAS | 400 | C38310 |
| | not yet been assigned. | | | |
| UNKNOWN | Route of administration is | UNKNOWN | 139 | C38311 |
| | unknown. | | | |
| URETERAL | Administration into the ureter. | URETER | 112 | C38312 |
| URETHRAL | Administration into the | URETH | 017 | C38271 |
| | urethra. | | | |
| VAGINAL | Administration into the | VAGIN | 015 | C38313 |
| | vagina. | | | |

In some embodiments, the route of administration may be oral, intranasal, inhalation, intravenous, sublingual, topical, injectable and/or transdermal.

The pharmaceutical compositions of the present invention comprising the TDP-43 modulator and/or metal channel activator may also include other pharmaceutically acceptable carriers and/or excipients such as binders, lubricants, diluents, coatings, disintegrants, barrier layer components, glidants, coloring agents, solubility enhancers, gelling agents, fillers, proteins, co-factors, emulsifiers, solubilizing agents, suspending agents and mixtures thereof. A skilled artisan in the art would know what other pharmaceutically acceptable carriers and/or excipients could be included in the formulations according to the invention. The choice of excipients would depend on the characteristics of the compositions and on the nature of other pharmacologically active compounds in the formulation.

The formulator will understand that excipients are used primarily to serve in delivering a safe, stable, and functional pharmaceutical. Excipients may also serve as part of the overall vehicle for delivery. Excipients may also be used to achieve effective absorption by the recipient of the active ingredient. An excipient may fill a role as simple and direct as being an inert filler, or an excipient as used herein may be part of a pH stabilizing system or coating. Excipients herein may also serve an antimicrobial function or improve pharmaceutical composition characteristics such as lubricity, flowability, disintegration and taste.

Pharmaceutical compositions may comprise one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Pharmaceutically acceptable carriers are biologically acceptable and compatible with the other ingredients in the formulation. Appropriate excipients are known to those skilled in the art (see Handbook of Pharmaceutical Excipients, fifth edition, 2005 edited by Rowe et al., McGraw Hill) and have been utilized to yield a novel sublingual formulation with unexpected properties. Pharmaceutical compositions herein are not limited to a single active ingredient, and supplementary active ingredients can also be incorporated.

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose; other carriers such as, polyvinylpyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinylpyrrolidone, agar, or alginic acid or a salt thereof such as sodium alginate. The compositions may be prepared by any of the methods of pharmacy but all methods include the step of bringing into association one or more chemical agents as described above with the carrier which constitutes one or more necessary ingredients. In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes, and the like. TDP-43 modulators, metal channel activator, and/or the pharmaceutically acceptable salts thereof can be formulated using pharmaceutically acceptable carriers well known in the art into dosages suitable for sublingual, intranasal or buccal administration. Such carriers enable the TDP-43 modulators and/or metal channel activator to be formulated in dosage forms such as tablets, powders, pills, capsules, liquids, gels, films, syrups, slurries, suspensions, and the like.

The TDP-43 modulator and/or metal channel activator compounds disclosed can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. Oral formulations containing a compound disclosed can be any conventionally used oral form, including but not limited to tablets, capsules, buccal forms, troches, lozenges and oral liquids, suspensions, or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato, or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidone, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, ion exchange resins, benzyl alcohol, eucalyptol, gelatin, limonene, mannitol, menthol, menthone, methyl acetate, sucralose, and vanillin. Surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions for oral, parenteral (such as intravenous, intramuscular, or other injections), or inhalation administration including but not limited to suspensions, emulsions, syrups, and elixirs. A TDP-43 modulator and/or metal channel activator compound of the present teachings can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal, or subcutaneous injection. Sterile injectable solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be further sub-divided to contain appropriate quantities of the compound. The unit dosage forms can be packaged compositions, for example, packeted powders, vials, ampoules, prefilled syringes, or sachets containing liquids. Alternatively, the unit dosage form can be a capsule or tablet itself, or it can be the appropriate number of any such compositions in package form. Such doses can be administered in any manner useful in directing the compound(s) to the recipient's bloodstream, including orally, via implants, parenterally (including intravenous, intraperitoneal, and subcutaneous injections), rectally, vaginally, and transdermally.

When administered for the treatment or inhibition of a particular disease state or disorder, it is understood that an effective dosage can vary depending upon the particular compound utilized, the pharmaceutical composition formulated, the mode of administration, physical factors related to the individual being treated, and severity of the condition being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition, stage of said condition, and the characteristics of the patient including their size, age and response pattern to the compound utilized.

In some cases, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can be sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

### Pharmacokinetics and Dosing

In some embodiments, a method of treating a neurological disease may be characterized by one or more pharmacokinetic parameters such as AUC, Cₘₐₓ, Tₘₐₓ, and others known and understood to persons of skill in the art. The term “pharmacokinetic” (PK) as used herein is used in its ordinary sense to mean the pharmacokinetic aspects of drug delivery. By definition pharmacokinetics (PK) is the study of how an organism affect a drug, e.g., how, and how fast it metabolizes the drug. The pharmacokinetics typically vary based upon the dosage amount of one or more of the disclosed TDP-43 modulators and/or metal channel activators. The pharmacokinetics may or may not vary as a function of administration route, and, if any, the binders, excipients, and dilutants in the pharmaceutical composition.

In some embodiments, a method of treating a neurological disease may be characterized by an AUC for a TDP-43 and/or metal channel activator compound according to Formulas 1-8, Formulas 200-206, and/or pharmaceutically acceptable salts thereof. In some embodiments, the AUC₀₋ₜ and/or AUC_{0-inf} (collectively referred to in the alternative as, simply, AUC) may be from about 80-125% of a given AUC value. In some embodiments, the AUC may be within 80-125% of about 25, 50, 100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 hr*ng/mL. In some embodiments, a systemic treatment may have a larger AUC than a localized (such as topical or subdermal) treatment.

In some embodiments, a method of treating a neurological disease may be characterized by a Cₘₐₓ for a TDP-43 and/or metal channel activator compound according to Formulas 1-8, Formulas 200-206, and/or pharmaceutically acceptable salts thereof. In some embodiments, the Cₘₐₓ may be from about 80-125% of a given Cₘₐₓ value. In some embodiments, the Cₘₐₓ may be within 80-125% of about 1, 2, 3, 4, 5, 8, 10, 20, 35, 50, 80,100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 ng/ml. In some embodiments, a systemic treatment may have a larger Cₘₐₓ than a localized (such as topical or subdermal) treatment.

In some embodiments, a ratio Cₘₐₓ/AUC may be used to characterize a method of treating a neurological disease wherein one or more of a TDP-43 and/or metal channel activator compound according to Formulas 1-8, Formulas 200-206, and/or pharmaceutically acceptable salts thereof are administered to a subject. In some embodiments, the Cₘₐₓ/AUC ratio may be from about 80-125% of a given Cₘₐₓ/AUC ratio. In some embodiments, the Cₘₐₓ/AUC ratio may be from about 80-125% of about 0.01, 0.03, 0.05, 0.08, 0.1, 0.13, 0.17, 0.2, 0.25, 0.3, 0.35, 0.4, 0.45, 0.5, 0.6, 0.7, 0.8, and 0.9.

In some embodiments, the Tₘₐₓ may range from about 0.1-16 hours, or from about 0.3-8 hours, or from about 0.5-4 hours, or from 0.5-2 hours, or from about 1-2 hours. In some embodiments, the route of administration, which may be any route described herein or known to a person of skill in the art, may affect the Tₘₐₓ of a compound according to Formulas 1-8, Formulas 200-206, and/or pharmaceutically acceptable salts thereof. Excipients or other pharmaceutically acceptable carriers in pharmaceutical compositions of one or more compounds according to Formulas 1-8, Formulas 200-206, and/or pharmaceutically acceptable salts thereof may alter the Tₘₐₓ value by making it larger or smaller than a pharmaceutical composition having the excipient or carrier excluded.

Solid compositions are normally formulated in dosage units providing from about 0.1 to about 1000 mg of the active ingredient per dose. Some examples of solid dosage units are 0.1 mg, 1 mg, 10 mg, 100 mg, 500 mg, and 1000 mg. Liquid compositions are generally in a unit dosage range of 0.1-100 mg/mL. Some examples of liquid dosage units are 0.1 mg/mL, 1 mg/mL, 10 mg/mL, 25 mg/mL, 50 mg/mL, and 100 mg/mL.

In some embodiments, a dose is daily. In some embodiments, a dose is twice daily. In some embodiments, a does is one, two, three, four, or five times daily. In some embodiments, a dose is once every other day, once every second day, once every third day, once every fourth day, once every fifth day, once every sixth day, weekly, bi-weekly, or monthly. Typically, a dose may be from 0.01-100 mg/kg body weight, or from 0.05-50 mg/kg body weight, or from 0.1-10 mg/kg body weight, or from 0.15-5 mg/kg body weight, or from 0.2-2 mg/kg body weight, or from 0.5-1.5 mg/kg body weight, or from 1-1.5 mg/kg body weight. In some embodiments, the dosing regimen may comprise one or more optional loading doses and a subsequent maintenance dose regimen. A loading dose may be larger than the doses given in a subsequent maintenance dose regimen.

In some embodiments, the dosage is adjusted based upon neurological disease symptoms observed in the patient. A symptom-dependent regimen may have a larger treatment dose while symptoms are present and a smaller preventative dose while symptoms are lessened, in remission, controlled, etc. In some embodiments, a treatment dose may comprise a unit dosage of about 10 mg, 20 mg, 30 mg, 40 mg, 50 mg, 60 mg, 70 mg, 75 mg, 80 mg, 100 mg, 120 mg, 140 mg, 160 mg, 180 mg, or 200 mg of a TDP-43 modulator according to any one of Formulas 1-8, and/or a metal channel activator according to any one of Formulas 200-206, and/or pharmaceutically acceptable salts thereof. In some embodiments, a preventative dose may comprise a unit dosage of about 1 mg, 2 mg, 5 mg, 10 mg, 20 mg, 30 mg, 40 mg, 50 mg, 60 mg, 70 mg, 75 mg, 80 mg, 100 mg, 120 mg, or 140 mg of a TDP-43 modulator according to any one of Formulas 1-8, and/or a metal channel activator according to any one of Formulas 200-206, and/or pharmaceutically acceptable salts thereof. In some embodiments, the preventative dose is an amount that is about 7/8, 3/4, 5/8, 1/2, 3/8, 1/4, or about ⅛ of the amount of the treatment dose. In some embodiments, if a patient receiving a preventative dosing regimen experiences recurrence or onset of symptoms, a treatment dosing regimen may replace the preventative dosing regimen until symptoms have lessened or subsided. Each of the treatment and/or preventative doses may be adjusted during the regimen based upon the severity of neurological disorder symptoms.

1. A pharmaceutical composition comprising a metal channel activator and a TDP-43 modulator.

2. The pharmaceutical composition of claim 1, wherein the metal channel activator is a potassium channel activator.

3. The pharmaceutical composition of claim 2, wherein the potassium channel activator is a Kv7 channel activator.

4. The pharmaceutical composition of claim 3, wherein the Kv7 channel activator is selected from the group consisting of a Kv7.1 channel activator, a Kv7.2 channel activator, a Kv7.3 channel activator, a Kv7.4 channel activator, a Kv7.5 channel activator, or any combination thereof.

5. The pharmaceutical composition of claim 3, wherein the Kv7 channel activator is a Kv7.2/7.3 channel activator.

6. The pharmaceutical composition of claim 1, wherein the metal channel activator is selected from one or more of the disclosed compounds according to any one or more of formulas 1 to 8.

7. The pharmaceutical composition of claim 1, wherein the TDP-43 modulator is selected from one or more of the disclosed compounds according to any one or more of formulas 200 to 206.

8. The pharmaceutical composition of claim 1, wherein the TDP-43 modulator is selected from one or more of the disclosed compounds according to any one or more of formulas 200 to 206, and wherein the metal channel activator is selected from one or more of the disclosed compounds according to any one or more of formulas 1 to 8.

9. The pharmaceutical composition of claim 1, wherein the TDP-43 modulator or a pharmaceutically acceptable salt thereof is in a form of a prodrug.

10. A method for treating or preventing a neurological disease that involves TDP-43, comprising administering a therapeutically effective amount of the pharmaceutical composition of claim 1 to a subject in need thereof.

11. The method of claim 10, wherein the disease is selected from the group consisting of amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), Alzheimer's disease (AD) related disorders, or any combination thereof.

12. A method for treating or preventing a disease associated with TDP-43 proteinopathies and/or that can benefit from Kv7 channel activation, comprising administering a therapeutically effective amount of the pharmaceutical composition of claim 1 to a subject in need thereof.

13. A method for treating or preventing diseases that involve excess amounts of TDP-43 in the cytosol, comprising administering a therapeutically effective amount of the pharmaceutical composition of claim 1 to a subject in need thereof.

14. A method of use of the TDP-43 modulators of the present invention as positron emission tomography (PET) imaging agents, comprising administering a therapeutically effective amount of the pharmaceutical composition of claim 1 to a subject in need thereof.

15. A method of use of the TDP-43 modulators of the present invention as single-photon emission computed tomography (SPECT) imaging agents, wherein the method comprises administering a therapeutically effective amount of the pharmaceutical composition of claim 1 comprising an isotopically labeled metal channel activator, an isotopically labeled TDP-43 modulator, or any combination thereof to a subject in need thereof.

16. A method for treating or preventing a disease that can benefit from Kv7 Channel activation, comprising administering a therapeutically effective amount of the pharmaceutical composition of claim 1 to a subject in need thereof.

17. A method for treating or preventing a disease associated with TDP-43 proteinopathies, comprising administering a therapeutically effective amount of the pharmaceutical composition of claim 1 to a subject in need thereof.

18. The method of claim 9, wherein the pharmaceutical composition is administered in a combination with at least one other agent known to treat amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), and Alzheimer's disease (AD) related disorders.

19-39. (canceled)

40. The method of claim 18, wherein the at least one other agent is riluzole, troriluzole, or edavarone.

41. A kit for treating a patient afflicted with a disorder involving metal channel dysfunction and TDP-43, said kit comprising:
(a) the metal channel activator or TDP-43 modulator; and
(b) instructions for administering said metal channel activator in combination with a TDP-43 modulator by the method of claim 10.

42. The kit of claim 41 wherein the patient is afflicted with a disorder involving metal channel dysfunction and TDP-43, said kit comprising:
(a) the metal channel activator; and
(b) instructions for administering said metal channel activator in combination with a TDP-43 modulator by the method of claim 10.

43. The kit of claim 41 wherein the patient is afflicted with a disorder involving metal channel dysfunction and TDP-43, said kit comprising:
(a) the TDP-43 modulator; and
(b) instructions for administering said TDP-43 modulator with a metal channel activator by the method of claim 10.

44. The pharmaceutical composition of claim 1, wherein the metal channel activator is the following compound, or a pharmaceutically acceptable salt thereof:

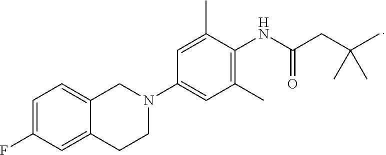 [SMILES: Cc1cc(N2CCc3cc(F)ccc3C2)cc(C)c1NC(=O)CC(C)(C)C]

45. The pharmaceutical composition of claim 1, wherein the metal channel activator is the following compound, or a pharmaceutically acceptable salt thereof:

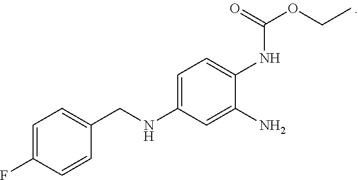 [SMILES: CCOC(=O)Nc1ccc(NCc2ccc(F)cc2)cc1N]

46. The pharmaceutical composition of claim 1, wherein the metal channel activator is a compound according to Formula 1, or a pharmaceutically acceptable salt thereof.

47. The pharmaceutical composition of claim 1, wherein the metal channel activator is a compound according to Formula 2, or a pharmaceutically acceptable salt thereof.

48. The pharmaceutical composition of claim 44, wherein the TDP-43 modulator is selected from one or more of the disclosed compounds according to any one or more of formulas 200 to 206.

49. A method for treating or preventing a neurological disease that involves TDP-43, comprising administering a therapeutically effective amount of the pharmaceutical composition of claim 48 to a subject in need thereof.

50. The method of claim 49, wherein the disease is selected from the group consisting of amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), Alzheimer's disease (AD) related disorders, or any combination thereof.

51. A method for treating or preventing a disease associated with TDP-43 proteinopathies and/or that can benefit from Kv7 channel activation, comprising administering a therapeutically effective amount of the pharmaceutical composition of claim 48 to a subject in need thereof.

52. A method for treating or preventing diseases that involve excess amounts of TDP-43 in the cytosol, comprising administering a therapeutically effective amount of the pharmaceutical composition of claim 48 to a subject in need thereof.

53. A method of use of the TDP-43 modulators of the present invention as positron emission tomography (PET) imaging agents, comprising administering a therapeutically effective amount of the pharmaceutical composition of claim 48 to a subject in need thereof.

54. A method of use of the TDP-43 modulators of the present invention as single-photon emission computed tomography (SPECT) imaging agents, wherein the method comprises administering a therapeutically effective amount of the pharmaceutical composition of claim 48, comprising an isotopically labeled metal channel activator, an isotopically labeled TDP-43 modulator, or any combination thereof to a subject in need thereof.

55. A method for treating or preventing a disease that can benefit from Kv7 Channel activation, comprising administering a therapeutically effective amount of the pharmaceutical composition of claim 48 to a subject in need thereof.

56. A method for treating or preventing a disease associated with TDP-43 proteinopathies, comprising administering a therapeutically effective amount of the pharmaceutical composition of claim 48 to a subject in need thereof.

57. The method of claim 49, wherein the pharmaceutical composition is administered in a combination with at least one other agent known to treat amyotrophic lateral sclerosis (ALS), frontotemporal lobar degeneration (FTLD), chronic traumatic encephalopathy (CTE), hippocampal sclerosis of aging (CARTS), inclusion body myositis (IBM), Alzheimer's disease (AD), and Alzheimer's disease (AD) related disorders.

58. The method of claim 57, wherein the at least one other agent is riluzole, troriluzole, or edavarone.

59. A kit for treating a patient afflicted with a disorder involving metal channel dysfunction and TDP-43, said kit comprising:
(a) the metal channel activator or TDP-43 modulator; and
(b) instructions for administering said metal channel activator in combination with a TDP-43 modulator by the method of claim 49.

60. The kit of claim 59 wherein the patient is afflicted with a disorder involving metal channel dysfunction and TDP-43, said kit comprising:
(a) the metal channel activator; and
(b) instructions for administering said metal channel activator in combination with a TDP-43 modulator by the method of claim 49.

61. The kit of claim 59 wherein the patient is afflicted with a disorder involving metal channel dysfunction and TDP-43, said kit comprising:
(a) the TDP-43 modulator; and
(b) instructions for administering said TDP-43 modulator with a metal channel activator by the method of claim 49.
